SEQUENCE LISTING (103 sequences)

SEQ ID NO: 1 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1260 aa, Severe acute respiratory syndrome-related coronavirus
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1259 aa, synthetic construct
QCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1258 aa, synthetic construct
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1260 aa, synthetic construct
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1257 aa, synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHQQAQSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1258 aa, synthetic construct
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGITAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1258 aa, synthetic construct
QCVNLTTRTQLPPAYTNSFTRGVYYPDKVFRSSVLHSTQDLFLPFFSNVTWFHVIHVSGTNGTKRFDNPVLPFNDGVYFASIEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINIVRDLPQGFSALEPLVDLPIGINITRFQTLLALHRSYLPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1257 aa, synthetic construct
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVSNHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1261 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVTSNHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATKFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 942 aa, synthetic construct
ITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 16 - protein, 1260 aa, Severe acute respiratory syndrome-related coronavirus; identical to SEQ ID NO: 2

SEQ ID NO: 17 - protein, 1250 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVALGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 1250 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 19 - protein, 1250 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - protein, 1266 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTHHHHHH

SEQ ID NO: 22 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - protein, 1257 aa, synthetic construct; identical to SEQ ID NO: 7

SEQ ID NO: 24 - protein, 1260 aa, synthetic construct
QCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - protein, 1259 aa, synthetic construct; identical to SEQ ID NO: 4

SEQ ID NO: 26 - protein, 1258 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - protein, 1255 aa, synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHGGSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 28 - protein, 1257 aa, synthetic construct
QCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 29 - protein, 1256 aa, synthetic construct
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 30 - protein, 1254 aa, synthetic construct
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 31 - protein, 1260 aa, synthetic construct; identical to SEQ ID NO: 6

SEQ ID NO: 32 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 33 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGGSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 34 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGGSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGNTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPQQAQSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 35 - protein, 1258 aa, synthetic construct; identical to SEQ ID NO: 9

SEQ ID NO: 36 - protein, 1257 aa, synthetic construct; identical to SEQ ID NO: 10

SEQ ID NO: 37 - protein, 1258 aa, synthetic construct; identical to SEQ ID NO: 5

SEQ ID NO: 38 - protein, 1260 aa, synthetic construct; identical to SEQ ID NO: 13

SEQ ID NO: 39 - protein, 1258 aa, synthetic construct; identical to SEQ ID NO: 8

SEQ ID NO: 40 - protein, 1260 aa, synthetic construct; identical to SEQ ID NO: 11

SEQ ID NO: 41 - protein, 1261 aa, synthetic construct; identical to SEQ ID NO: 12

SEQ ID NO: 42 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 43 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 44 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNNLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTVSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 45 - protein, 1257 aa, synthetic construct
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 46 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVRQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 47 - protein, 1257 aa, synthetic construct; identical to SEQ ID NO: 10

SEQ ID NO: 48 - protein, 1257 aa, synthetic construct; identical to SEQ ID NO: 42

SEQ ID NO: 49 - protein, 1255 aa, synthetic construct; identical to SEQ ID NO: 43

SEQ ID NO: 50 - protein, 1255 aa, synthetic construct; identical to SEQ ID NO: 44

SEQ ID NO: 51 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 52 - protein, 1257 aa, synthetic construct; identical to SEQ ID NO: 46

SEQ ID NO: 53 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNSSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 54 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 55 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSKLKPFERDISTEIYQAGNKPCNGVAGSNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNISNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLINLQELGKYEQYIKWPWYIWLGFIAGLIAIVMVTIMLCCMTSCCSCLKGCCSCGSCCKFDEDDSEPVLKGVKLHYT

SEQ ID NO: 56 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 57 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 58 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 59 - protein, 1254 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 60 - protein, 1256 aa, synthetic construct
QCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 61 - protein, 1254 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 62 - protein, 1257 aa, synthetic construct
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHXNNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRTRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 63 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 64 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 65 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNSSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 66 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSKLKPFERDISTEIYQAGNKPCNGVAGSNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNISNQVAVLYQGVNCTEVPVAIHADQLTPTWRVYSTGSNVFQTRAGCLIGAEYVNNSYECDIPIGAGICASYQTQTKSHQQAQSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLINLQELGKYEQYIKWPWYIWLGFIAGLIAIVMVTIMLCCMTSCCSCLKGCCSCGSCCKFDEDDSEPVLKGVKLHYT

SEQ ID NO: 67 - protein, 4 aa, Severe acute respiratory syndrome-related coronavirus
RRAR

SEQ ID NO: 68 - protein, 4 aa, synthetic construct
QQAQ

SEQ ID NO: 69 - protein, 4 aa, synthetic construct
QRAR

SEQ ID NO: 70 - protein, 4 aa, synthetic construct
RQAR

SEQ ID NO: 71 - protein, 4 aa, synthetic construct
RRAQ

SEQ ID NO: 72 - protein, 4 aa, synthetic construct
QQAR

SEQ ID NO: 73 - protein, 4 aa, synthetic construct
RQAQ

SEQ ID NO: 74 - protein, 4 aa, synthetic construct
QRAQ

SEQ ID NO: 75 - protein, 4 aa, synthetic construct
NNAN

SEQ ID NO: 76 - protein, 4 aa, synthetic construct
NRAR

SEQ ID NO: 77 - protein, 4 aa, synthetic construct
RNAR

SEQ ID NO: 78 - protein, 4 aa, synthetic construct
RRAN

SEQ ID NO: 79 - protein, 4 aa, synthetic construct
NNAR

SEQ ID NO: 80 - protein, 4 aa, synthetic construct
RNAN

SEQ ID NO: 81 - protein, 4 aa, synthetic construct
NRAN

SEQ ID NO: 82 - protein, 4 aa, synthetic construct
AAAA

SEQ ID NO: 83 - protein, 4 aa, synthetic construct
ARAR

SEQ ID NO: 84 - protein, 4 aa, synthetic construct
RAAR

SEQ ID NO: 85 - protein, 4 aa, synthetic construct
RRAA

SEQ ID NO: 86 - protein, 4 aa, synthetic construct
AAAR

SEQ ID NO: 87 - protein, 4 aa, synthetic construct
RAAA

SEQ ID NO: 88 - protein, 4 aa, synthetic construct
ARAA

SEQ ID NO: 89 - protein, 4 aa, synthetic construct
GGAG

SEQ ID NO: 90 - protein, 4 aa, synthetic construct
GRAR

SEQ ID NO: 91 - protein, 4 aa, synthetic construct
RGAR

SEQ ID NO: 92 - protein, 4 aa, synthetic construct
RRAG

SEQ ID NO: 93 - protein, 4 aa, synthetic construct
GGAR

SEQ ID NO: 94 - protein, 4 aa, synthetic construct
RGAG

SEQ ID NO: 95 - protein, 4 aa, synthetic construct
GRAG

SEQ ID NO: 96 - protein, 4 aa, synthetic construct
GSAS

SEQ ID NO: 97 - protein, 4 aa, synthetic construct
GSGA

SEQ ID NO: 98 - DNA, 4829 bp, synthetic construct
  source 1..4829: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattccgcgggcggccgcgaaatggagccagtagatcctagactagagccctggaagcatccaggaagtcagcctaaaactgcttgtaccacttgctattgtaaaaagtgttgctttcattgccaagtttgtttcacaacaaaagccttaggcatctcctatggcaggaagaagcggagacagcgacgaagacctcctcaaggcagtcagactcatcaagtttctctatcaaagcaacccacctcccaaccccgaggggacccgacaggcccgaaggaataggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 99 - DNA, 8910 bp, synthetic construct
  source 1..8910: mol_type = other DNA
agcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagctgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattctagactgccatgggcgcccgcgcctccgtgctgtccggcggcgagctggacaagtgggagaagatccgcctgcgccccggcggcaagaagcagtacaagctgaagcacatcgtgtgggcctcccgcgagctggagcgcttcgccgtgaaccccggcctgctggagacctccgagggctgccgccagatcctgggccagctgcagccctccctgcaaaccggctccgaggagctgcgctccctgtacaacaccatcgccgtgctgtactgcgtgcaccagcgcatcgacgtgaaggacaccaaggaggccctggacaagatcgaggaggagcagaacaagtccaagaagaaggcccagcaggccgccgccgacaccggcaacaactcccaggtgtcccagaactaccccatcgtgcagaacctgcagggccagatggtgcaccaggccatctccccccgcaccctgaacgcctgggtgaaggtggtggaggagaaggccttctcccccgaagtcatccccatgttctccgccctgtccgagggcgccaccccccaggacctgaacaccatgctgaacaccgtgggcggccaccaggccgccatgcagatgctgaaggagaccatcaacgaggaggccgccgagtgggaccgcctgcaccccgtgcacgccggccccatcgcccccggccagatgcgcgagccccgcggctccgacatcgccggcaccacctccaccctgcaagagcagatcggctggatgacccacaacccccccatccccgtgggcgagatctacaagcgctggatcatcctgggcctgaacaagatcgtgcgcatgtactcccccacctccatcctggacatccgccagggccccaaggagcccttccgcgactacgtggaccgcttctacaagaccctgcgcgccgagcaggcctcccaggaggtaaagaactggatgaccgagaccctgctggtgcagaacgccaaccccgactgcaagaccatcctgaaggccctgggccccggcgccaccctggaggagatgatgaccgcctgccagggcgtgggcggccccggccacaaggcccgcgtgctggccgaggccatgtcccaagtcaccaaccccgccaccatcatgatccagaagggcaacttccgcaaccagcgcaagaccgtgaagtgcttcaactgcggcaaggagggccacatcgccaagaactgccgcgccccccgcaagaagggctgctggaagtgcggcaaggagggccaccagatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagatcggtggccagctgaaggaggccctgctggacaccggcgccgacgacaccgtgctggaggagatgaacctgcccggccgctggaagcccaagatgatcggcggcatcggcggcttcatcaaagtccgccagtacgaccagatcctgatcgagatctgcggccacaaggccatcggcaccgtgctggtgggccccacccccgtgaacatcatcggccgcaacctgctgacccagatcggctgcaccctgaacttccccatctcccccatcgagaccgtgcccgtgaagctgaagcccggcatggacggccccaaagtcaagcagtggcccctgaccgaggagaagatcaaggccctggtggagatctgcaccgagatggagaaggagggcaagatctccaagatcggccccgagaacccctacaacacccccgtgttcgccatcaagaagaaggactccaccaagtggcgcaagctggtggacttccgcgagctgaacaagcgcacccaggacttctgggaggtgcagctgggcatcccccaccccgccggcctgaagcagaagaagtccgtgaccgtgctggacgtgggcgacgcctacttctccgtgcccctggacaaggacttccgcaagtacaccgccttcaccatcccctccatcaacaacgagacccccggcatccgctaccagtacaacgtgctgccccagggctggaagggctcccccgccatcttccagtgctccatgaccaagatcctggagcccttccgcaagcagaaccccgacatcgtgatctaccagtacatggacgacctgtacgtgggctccgacctggagatcggccagcaccgcaccaagatcgaggagctgcgccagcacctgctgcgctggggcttcaccacccccgacaagaagcaccagaaggagccccccttcctgtggatgggctacgagctgcaccccgacaagtggaccgtgcagcccatcgtgctgcccgagaaggactcctggaccgtgaacgacatccagaagctggtgggcaagctgaactgggcctcccagatctacgccggcatcaaagtccgccagctgtgcaagctgctgcgcggcaccaaggccctgaccgaggtggtgcccctgaccgaggaggccgagctggagctggccgagaaccgcgagatcctgaaggagcccgtgcacggcgtgtactacgacccctccaaggacctgatcgccgagatccagaagcagggccagggccagtggacctaccagatctaccaggagcccttcaagaacctgaagaccggcaaatacgcccgcatgaagggcgcccacaccaacgacgtgaagcagctgaccgaggccgtgcagaagatcgccaccgagtccatcgtgatctggggcaagactcccaagttcaagctgcccatccagaaggagacctgggaggcctggtggaccgagtactggcaggccacctggatccccgagtgggagttcgtgaacaccccccccctggtgaagctgtggtaccagctggagaaggagcccatcatcggcgccgagaccttctacgtggacggcgccgccaaccgcgagaccaagctgggcaaggccggctacgtgaccgaccgcggccgccagaaggtggtgcccctgaccgacaccaccaaccagaagaccgagctgcaggccatccacctggccctgcaagactccggcctggaggtgaacatcgtgaccgactcccagtatgcattgggcatcatccaggcccagcccgacaagtccgagtccgagctggtgtcccagatcatcgagcagctgatcaagaaggagaaggtgtacctggcctgggtgcccgcccacaagggcatcggcggcaacgagcaggtggacaagctggtgtccgccggcatccgcaaggtgctgttcctggacggcatcgacaaggcccaggaggagcacgagaagtaccactccaactggcgcgccatggcctccgacttcaacctgccccccgtggtggccaaggagatcgtggcctcctgcgacaagtgccagctgaagggcgaggccatgcacggccaggtggactgctcccccggcatctggcagctggactgcacccacctggagggcaaggtgatcctggtggccgtgcacgtggcctccggctacatcgaggccgaggtgatccccgccgagaccggccaggagaccgcctacttcctgctgaagctggccggccgctggcccgtgaagaccgtgcacaccgacaacggctccaacttcacctccaccaccgtgaaggccgcctgctggtgggccggcatcaagcaggagttcggcatcccctacaacccccagtcccagggcgtgatcgagtccatgaacaaggagctgaagaagatcatcggccaagtccgcgaccaggccgagcacctgaagaccgccgtgcagatggccgtgttcatccacaacttcaagcgcaagggcggcatcggcggctactccgccggcgagcgcatcgtggacatcatcgccaccgacatccagaccaaggagctgcagaagcagatcaccaagatccagaacttccgcgtgtactaccgcgactcccgcgaccccgtgtggaagggccccgccaagctgctgtggaagggcgagggcgccgtggtgatccaggacaactccgacatcaaggtggtgccccgccgcaaggccaagatcatccgcgactacggcaagcagatggccggcgacgactgcgtggcctcccgccaggacgaggactaacacatggaaaagattagtaaaacaccataggccgctctagaggatccaagcttatcgataccgtcgacctcgagggcccagatctaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacggagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcggggagctttttgcaaaagcctaggcctccaaaaaagcctcctcactacttctggaatagctcagaggccgaggcggcctcggcctctgcataaataaaaaaaattagtcagccatg

SEQ ID NO: 100 - DNA, 5900 bp, synthetic construct
  source 1..5900: mol_type = other DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagcttggtaccgagctcggatccactagtaacggccgccagtgtgctggaattctgcagatatccatcacactggcggccgctcgagcatgcatctagactgccatggcaggaagaagcggagacagcgacgaagacctcctcaaggcagtcagactcatcaagtttctctatcaaagcaacccacctcccaaccccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaataggatccggctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattttaacaaaatattaacgtttacaatttaaatatttgcttatacaatcttcctgtttttggggcttttctgattatcaaccggggtgggtaccgagctcgaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttggatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctggatcccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 101 - DNA, 9394 bp, synthetic construct
  source 1..9394: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcggccgcatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagacaagatcacctgcaggacaggcgcgccctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcacccgggcgattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 102 - protein, 805 aa, Homo sapiens [sequence omitted]

SEQ ID NO: 103 - protein, 788 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

(19) United States
(12) Patent Application Publication
(10) Pub. No.: US 2026/0028644 A1
(43) Pub. Date: Jan. 29, 2026

(54) PSEUDOVIRUS BASED NEUTRALIZATION ASSAY FOR EVALUATING VACCINE IMMUNOGENICITY

(71) Applicant: NOVAVAX, INC., Gaithersburg, MD (US)

(72) Inventors: Zhaohui CAI, Gaithersburg, MD (US); Raj KALKERI, Gaithersburg, MD (US)

(21) Appl. No.: 18/998,469

(22) PCT Filed: Jul. 26, 2023
(86) PCT No.: PCT/US2023/071047

§ 371 Date: Jan. 25, 2025

Related U.S. Application Data
(60) Provisional application No. 63/392,397, filed on Jul. 26, 2022.
(60) Provisional application No. 63/407,371, filed on Sep. 16, 2022.

(51) Int. Cl. C12N 15/86; C07K 14/005; C12Q 1/6897; C12Q 1/70

(52) U.S. Cl. CPC C12N 15/86; C07K 14/005; C12Q 1/6897; C12Q 1/70; C12N 2740/00043; C12N 2740/00051; C12N 2770/20022

(57) ABSTRACT

Provided herein are pseudoviruses expressing a SARS-CoV-2 S glycoprotein. Also provided herein are assays that employ the pseudoviruses to evaluate the immunogenicity of a biological sample against a SARS-CoV-2 virus or variant thereof. Also provided herein are methods of evaluating the immunogenicity of a COVID-19 vaccine using the assays.

### CROSS-REFERENCE TO RELATED APPLICATIONS

This application claims priority to U.S. Provisional Application No. 63/392,397, filed on Jul. 26, 2022, and U.S. Provisional Application No. 63/407,371, filed on Sep. 16, 2022. These applications are hereby incorporated by reference in their entirety for all purposes.

### REFERENCE TO AN ELECTRONIC SEQUENCE LISTING

The contents of the electronic sequence listing (NOVV-101_02WO_SeqList_ST26.xml; Size: 203,198 bytes; and Date of Creation: Jul. 26, 2023) are herein incorporated by reference in its entirety.

### FIELD

The present disclosure is generally related to pseudoviruses expressing a SARS-CoV-2 S glycoprotein. The disclosure is also related to assays that employ the pseudoviruses to evaluate the immunogenicity of a biological sample (e.g., whether the biological sample contains neutralizing antibodies) against a SARS-CoV-2 virus or variant thereof. The disclosure is also related to methods of evaluating the ability of COVID-19 vaccines to stimulate an immune response against a SARS-CoV-2 virus or variant thereof using the assays.

### BACKGROUND OF THE INVENTION

The neutralizing antibody response elicited by COVID-19 vaccines plays a pivotal role in conferring protection against severe acute respiratory syndrome coronavirus 2 (SARS-CoV-2) virus. To measure neutralizing antibodies against SARS-CoV-2 viruses, biosafety level 3 (BSL-3) laboratories must be used. BSL-3 laboratories require more stringent laboratory safety precautions compared to BSL-2 laboratories. These include restricted/controlled lab access, medical surveillance and/or immunizations for personnel, proper lab design (including self-closing and locking doors, sustained directional airflow, and sink/eyewash stations), use of proper biosafety cabinets, and proper personal protection equipment (which may include respirators).

As a result of these safety precautions, BSL-3 laboratories are expensive to operate and difficult to get access to. There is a need in the art for novel assays that enable the measurement of SARS-CoV-2 neutralizing antibodies outside of a BSL-3 laboratory.

### SUMMARY OF THE INVENTION

Provided herein is a pseudovirus-based neutralization assay for measuring neutralizing antibodies against SARS-CoV-2 viruses. The assay described herein can be performed in a biosafety level 2 (BSL-2) laboratory. Thus, the assay is more cost effective than conventional assays that require the use of SARS-CoV-2 viruses.

Provided herein is a pseudovirus encoding: (i) a SARS-CoV-2 Spike (S) glycoprotein; (ii) one or more of Rev, Tat, Gag, and Pol proteins; and (iii) a reporter protein.

Provided herein is a method of producing a pseudovirus, comprising transfecting cells with: (i) a plasmid encoding a lentiviral backbone and a reporter protein; (ii) one or more plasmids encoding a Rev protein, a Tat protein, a Gag protein, a Pol protein; or a combination thereof; and (iii) a plasmid expressing a SARS-CoV-2 Spike (S) glycoprotein.

Provided herein is a method for measuring the ability of a biological sample to stimulate an immune response against a SARS-CoV-2 virus or a variant thereof, comprising:
(a) contacting a biological sample with a pseudovirus expressing a SARS-CoV-2 S glycoprotein and a reporter protein;
(b) contacting cells expressing angiotensin converting enzyme 2 with the pseudovirus; and
(c) quantifying expression of the reporter protein.

Provided herein is a pseudovirus encoding: (i) a SARS-CoV-2 Spike (S) glycoprotein; (ii) one or more of Rev, Tat, Gag, and Pol proteins; and (iii) a reporter protein. In embodiments, the pseudovirus encodes from 1 to about 20, from 2 to about 20, from 3 to about 20, from 4 to about 20, from 5 to about 20, from 6 to about 20, from 7 to about 20, from 8 to about 20, from 9 to about 20, from about 10 to about 20, from about 11 to about 20, from about 12 to about 20, from about 13 to about 20, from about 14 to about 20, from about 15 to about 20, from about 16 to about 20, from about 17 to about 20, from about 18 to about 20, from about 19 to about 20, from 1 to about 10, from 2 to about 10, from 3 to about 10, from 4 to about 10, from 5 to about 10, from 6 to about 10, from about 7 to about 10, from about 8 to about 10, from 2 to 5, from 3 to 5, from 4 to about 8, from 5 to about 8, or from about 6 to about 8 SARS-CoV-2 S glycoproteins. In embodiments, the reporter protein is luciferase. In embodiments, the reporter protein is a fluorescent protein. In embodiments, the fluorescent protein is ZsGreen. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 1, SEQ ID NO: 2, or SEQ ID NO: 3. In embodiments, the SARS-CoV-2 S glycoprotein has an inactive furin cleavage site. In embodiments, the SARS-CoV-2 S glycoprotein has an inactive furin cleavage site having the amino acid sequence of QQAQ (SEQ ID NO: 68). In embodiments, amino acids 973 and 974 of the SARS-CoV-2 S glycoprotein are proline, as compared to a wild-type SARS-CoV-2 S glycoprotein having the amino acid sequence of SEQ ID NO: 2. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3, 5-13, 15, 17-19, 21, and 23-66. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3-13, 20, and 22-66. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3, 5-13, and 23-66. In embodiments, the SARS-CoV-2 S glycoprotein is from a SARS-CoV-2 virus or a variant of SARS-CoV-2. In embodiments, the variant of SARS-CoV-2 is a B.1.1.7 SARS-CoV-2 strain; a B.1.351 SARS-CoV-2 strain; a P.1 SARS-CoV-2 strain; a Cal.20C SARS-CoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; a C.37 SARS-CoV-2 strain; a B.1.621 SARS-CoV-2 strain; or a B.1.1.529 SARS-CoV-2 strain. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a SARS-CoV-2 S glycoprotein from a SARS-CoV-2 S omicron variant selected from the group consisting of: BA.1, BA.2.12.1, BA.2, BA.3, BA.4, BA.5, XBB.1.5, XBB.2.3, and XBB.1.16. In embodiments, the pseudovirus comprises a Gag protein and a Pol protein. In embodiments, a fusion protein encodes the Gag protein and Pol protein. In embodiments, the Gag protein and Pol protein are from a Maloneymurine Leukemia Virus (MLV). In embodiments, the Rev, Tat, Gag, and Pol proteins are from a lentivirus.

In embodiments, provided herein is a method of producing a pseudovirus described herein, comprising transfecting a host cell with: (i) a transfer plasmid encoding a reporter protein; (ii) one or more plasmids encoding a Rev protein, a Tat protein, a Gag protein, a Pol protein; or a combination thereof; and (iii) a plasmid encoding a SARS-CoV-2 Spike (S) glycoprotein. In embodiments, the host cell is a human cell. In embodiments, the host cell is a HEK293T (also called “293T” cell).

In embodiments, provided herein is a method for determining if a biological sample contains neutralizing antibodies against a SARS-CoV-2 virus or a variant thereof, comprising: (a) contacting a biological sample with from 1 to about 20 pseudoviruses as described herein; (b) contacting a cell expressing angiotensin converting enzyme 2 (ACE2) with the from 1 to about 20 pseudoviruses; (c) quantifying expression of the reporter protein in the cell expressing ACE2; (d) contacting a control cell expressing ACE2 with the from 1 to about 20 pseudoviruses, wherein the from 1 to about 20 pseudoviruses have not been contacted with the biological sample; and (e) quantifying expression of the reporter protein in the control cell expressing ACE2; wherein if the expression of the reporter protein in the cell of (c) is less than expression of the reporter protein in the control cell of (e), then the biological sample contains neutralizing antibodies against the SARS-CoV-2 virus or the variant thereof. In embodiments, the method comprises contacting the biological sample and pseudovirus for from 1 hour to about 7 hours. In embodiments, the method comprises contacting the biological sample and pseudovirus for about two hours. In embodiments, the method comprises contacting the biological sample and pseudovirus at about 37° C. In embodiments, the method comprises heat inactivating the biological sample. In embodiments, the method comprises contacting the cell with pseudovirus for from 1 day to about 7 days. In embodiments, the method comprises contacting the cell with pseudovirus for about 3 days. In embodiments, the cells are selected from the group consisting of: Vero E6, A549, and HEK293T cells. In embodiments, the cells are HEK293T cells. In embodiments, performing the method does not require a biosafety level 3 (BSL-3) laboratory. In embodiments, the biological sample is saliva, a nasopharyngeal swab, sputum, saliva, urine, a fecal sample, cerebrospinal fluid, synovial fluid, serum, blood, a mucosal sample, or plasma. In embodiments, the biological sample is from a patient that has been administered an immunogenic composition against a SARS-CoV-2 virus or a variant thereof. In embodiments, the biological sample is from a patient that has previously had COVID-19. In embodiments, the method comprises contacting the biological sample with about 1, about 2, about 3, about 4, about 5, about 6, about 7, about 8, about 9, about 10, about 11, about 12, about 13, about 14, about 15, about 16, about 17, about 18, about 19, or about 20 pseudoviruses. In embodiments, each pseudovirus expresses a different reporter gene and a different SARS-CoV-2 S glycoprotein.

### BRIEF DESCRIPTION OF THE DRAWINGS

FIG. 1 provides a schematic of the pseudovirus neutralization assay of Example 1.

FIGS. 2A-2B show the dependence of luminescence readout on the amount of pseudovirus used for infection in the pseudovirus neutralization assay of Example 1. FIG. 2A shows the dependence of luminescence readout on the amount of pseudovirus used for infection when the pseudovirus expresses a SARS-CoV-2 S protein from the prototype strain. FIG. 2B shows the dependence of luminescence readout on the amount of pseudovirus used for infection when the pseudovirus expresses a SARS-CoV-2 S protein from the Omicron BA.1, BA.2, or BA. 5 variant. HEK293T cells were infected with serial dilutions of pseudoviruses expressing either the SARS-CoV-2 S protein of the prototype SARS-CoV-2 strain or the SARS-CoV-2 S protein of a SARS-CoV-2 variant (Omicron BA.1, BA.2, BA.5), followed by measuring the luciferase levels after 2 days post-infection. Dose dependent luciferase levels with decreasing amounts of pseudoviruses used for infection are shown in the figure.

FIGS. 3A-3C show the ability of the pseudoviruses of Example 1 to infect A549 cells (FIG. 3A), 293T cells (FIG. 3B), and Vero E6 cells (FIG. 3C).

FIGS. 4A-4B show the ID50 of pseudovirus infection for 293T cells and A549/ACE2-TMPRESS cells. The ID50 of Pseudovirus infection in FIG. 4A is for pseudovirus expressing the Omicron BA.1 S protein. The ID50 of Pseudovirus infection in FIG. 4B is for Pseudovirus expressing the Omicron BA.5 S protein.

FIGS. 5A-5B show the luminescence output 48 hours (FIG. 5A) and 72 hours (FIG. 5B) after HEK293T cells expressing ACE2 are infected with pseudovirus in the experiment described in Example 1.

FIGS. 6A-6C shows the inter-and intra-assay precision of the pseudovirus neutralization assay of Example 1. FIG. 6A shows precision when the pseudovirus encodes a SARS-CoV-2 S protein from the prototype strain. FIG. 6B shows precision when the pseudovirus encodes a SARS-CoV-2 S protein from the Omicron BA. 1 strain. FIG. 6C shows precision when the pseudovirus encodes a SARS-CoV-2 S protein from the Omicron BA.5 strain.

FIGS. 7A-7B shows the linearity of the pseudovirus based neutralization assay of Example 1 for two different operators, Operator #1 (FIG. 7A) and Operator #2 (FIG. 7B).

FIG. 8 shows that results from the pseudovirus neutralization assay of Example 1 are significantly correlated for prototype/Wuhan strain with the live virus MN assay (Pearson's r=0.9304, R²=0.8657, P<0.0001). Test serum samples (n=13) from a clinical study were evaluated in PNT against prototype/Wuhan strain as per the method mentioned above, followed by regression analysis with a validated live virus microneutralization assay. Analysis was performed using GraphPad Prism software (9.3.1). Dotted line shows 95% confidence interval (CI). Pearson correlation coefficient (Pearson r) and two tailed P values are shown.

FIGS. 9A-9B show that the ability of a serum sample to resist pseudovirus infection is correlated with the amount of anti-SARS-CoV-2 S protein immunoglobulin (FIG. 9A) and hACE2 binding inhibition (FIG. 9B).

FIGS. 10A-10C show the neutralization titers (ID50) for serum from patients 1 and 2 (FIG. 10A, patients 3 and 4 (FIG. 10B), and patients 6 and 7 (FIG. 10C). FIG. 10D shows the neutralization titers of patient #5 14 days after administration of a primary NVX-CoV2373 vaccine series (“2x” in FIG. 10D) and after administration of a NVX-CoV2373 booster (“3x” in FIG. 10D).

FIGS. 11A-11D show the fluorescence (Average+/−Standard deviation from duplicate wells) of 293T cells expressing ACE2 that have been infected with a pseudovirus expressing a SARS-CoV-2 S glycoprotein from an Omicron BA.1 strain and a red fluorescent protein (“BA.1 RFP”), a pseudovirus expressing a SARS-CoV-2 S glycoprotein from an Omicron BA.5 strain and a green fluorescent protein (“BA.5 GFP”), or both of the pseudoviruses (“Dual adj”). FIG. 11A shows the red fluorescent protein (RFP) fluorescence of the cells, as determined by a fluorescent plate reader (CELIGO). FIG. 11B shows the green fluorescent protein (GFP) fluorescence of the cells, as determined by a fluorescent plate reader (CELIGO). FIG. 11C shows the red fluorescent protein (RFP) fluorescence of the cells, as determined by a fluorescent plate reader (SpectraMax® iD3). FIG. 11D shows the green fluorescent protein (GFP) fluorescence of the cells, as determined by a fluorescent plate reader (SpectraMax® iD3).

FIGS. 12A-12B show linear regression plots of pseudoneutralization titers from two samples, wherein the pseudovirus expresses a SARS-CoV-2 S glycoprotein having a D614G mutation, as compared to a SARS-CoV-2 S glycoprotein of SEQ ID NO: 2.

FIGS. 13A-13B show linear regression plots of pseudoneutralization titers from two samples, wherein the pseudovirus expresses a SARS-CoV-2 S glycoprotein from the SARS-CoV-2 Omicron BA.5 strain.

FIGS. 14A-14B show linear regression plots of pseudoneutralization titers from two samples, wherein the pseudovirus expresses a SARS-CoV-2 S glycoprotein from the SARS-CoV-2 Omicron XBB.1.5 strain.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

As used herein, the term “about” or “approximately” when preceding a numerical value indicates the value plus or minus a range of 10%. For example, “about 100” encompasses 90 and 110.

As used herein, the terms “immunogen,” “antigen,” and “epitope” refer to substances such as proteins, including glycoproteins, and peptides that are capable of eliciting an immune response.

As used herein, an “immunogenic composition” is a composition that comprises an antigen where administration of the composition to a subject results in the development in the subject of a humoral and/or a cellular immune response to the antigen.

As used herein, the term “vaccine” refers to an immunogenic composition, such as an immunogen derived from a pathogen, which is used to induce an immune response against the pathogen that provides protective immunity (e.g., immunity that protects a subject against infection with the pathogen and/or reduces the severity of the disease or condition caused by infection with the pathogen). The protective immune response may include formation of antibodies and/or a cell-mediated response. Depending on context, the term “vaccine” may also refer to a suspension or solution of an immunogen that is administered to a subject to produce protective immunity.

As used herein, the term “modification” as it refers to a CoV S polypeptide refers to mutation, deletion, or addition of one amino acid of the CoV S polypeptide. The location of a modification within a CoV S polypeptide can be determined based on aligning the sequence of the polypeptide to SEQ ID NO: 1 (CoV S polypeptide containing signal peptide) or SEQ ID NO: 2 (mature CoV S polypeptide lacking a signal peptide). (see below sequences).

| SEQ ID NO: | Amino Acid Sequence |
|---|---|
| 1 | MFVFLVLLPLVSSQCVNLTTRTQLPPAYTNSFTRGVYYPDKVFRSSVL |
| | HSTQDLFLPFFSNVTWFHAIHVSGTNGTKRFDNPVLPFNDGVYFASTE |
| | KSNIIRGWIFGTTLDSKTQSLLIVNNATNVVIKVCEFQFCNDPFLGVYY |
| | HKNNKSWMESEFRVYSSANNCTFEYVSQPFLMDLEGKQGNFKNLRE |
| | FVFKNIDGYFKIYSKHTPINLVRDLPQGFSALEPLVDLPIGINITRFQTLL |
| | ALHRSYLTPGDSSSGWTAGAAAYYVGYLQPRTFLLKYNENGTITDAV |
| | DCALDPLSETKCTLKSFTVEKGIYQTSNFRVQPTESIVRFPNITNLCPFG |
| | EVFNATRFASVYAWNRKRISNCVADYSVLYNSASFSTFKCYGVSPTK |
| | LNDLCFTNVYADSFVIRGDEVRQIAPGQTGKIADYNYKLPDDFTGCVI |
| | AWNSNNLDSKVGGNYNYLYRLFRKSNLKPFERDISTEIYQAGSTPCN |
| | GVEGFNCYFPLQSYGFQPTNGVGYQPYRVVVLSFELLHAPATVCGPK |
| | KSTNLVKNKCVNFNFNGLTGTGVLTESNKKFLPFQQFGRDIADTTDA |
| | VRDPQTLEILDITPCSFGGVSVITPGTNTSNQVAVLYQDVNCTEVPVAI |
| | HADQLTPTWRVYSTGSNVFQTRAGCLIGAEHVNNSYECDIPIGAGICA |
| | SYQTQTNSPRRARSVASQSIIAYTMSLGAENSVAYSNNSIAIPTNFTISV |
| | TTEILPVSMTKTSVDCTMYICGDSTECSNLLLQYGSFCTQLNRALTGIA |
| | VEQDKNTQEVFAQVKQIYKTPPIKDFGGFNFSQILPDPSKPSKRSFIED |
| | LLFNKVTLADAGFIKQYGDCLGDIAARDLICAQKFNGLTVLPPLLTDE |
| | MIAQYTSALLAGTITSGWTFGAGAALQIPFAMQMAYRENGIGVTQNV |
| | LYENQKLIANQFNSAIGKIQDSLSSTASALGKLQDVVNQNAQALNTL |
| | VKQLSSNFGAISSVLNDILSRLDKVEAEVQIDRLITGRLQSLQTYVTQQ |
| | LIRAAEIRASANLAATKMSECVLGQSKRVDFCGKGYHLMSFPQSAPH |
| | GVVFLHVTYVPAQEKNFTTAPAICHDGKAHFPREGVFVSNGTHWFVT |
| | QRNFYEPQIITTDNTFVSGNCDVVIGIVNNTVYDPLQPELDSFKEELDK |
| | YFKNHTSPDVDLGDISGINASVVNIQKEIDRLNEVAKNLNESLIDLQEL |
| | GKYEQYIKWPWYIWLGFIAGLIAIVMVTIMLCCMTSCCSCLKGCCSC |
| | GSCCKFDEDDSEPVLKGVKLHYT |
| 2 | QCVNLTTRTQLPPAYTNSFTRGVYYPDKVFRSSVLHSTQDLFLPFFSN |
| | VTWFHAIHVSGTNGTKRFDNPVLPFNDGVYFASTEKSNIIRGWIFGTT |
| | LDSKTQSLLIVNNATNVVIKVCEFQFCNDPFLGVYYHKNNKSWMESE |
| | FRVYSSANNCTFEYVSQPFLMDLEGKQGNFKNLREFVFKNIDGYFKIY |
| | SKHTPINLVRDLPQGFSALEPLVDLPIGINITRFQTLLALHRSYLTPGDS |
| | SSGWTAGAAAYYVGYLQPRTFLLKYNENGTITDAVDCALDPLSETKC |
| | TLKSFTVEKGIYQTSNFRVQPTESIVRFPNITNLCPFGEVFNATRFASVY |
| | AWNRKRISNCVADYSVLYNSASFSTFKCYGVSPTKLNDLCFTNVYAD |
| | SFVIRGDEVRQIAPGQTGKIADYNYKLPDDFTGCVIAWNSNNLDSKV |
| | GGNYNYLYRLFRKSNLKPFERDISTEIYQAGSTPCNGVEGFNCYFPLQ |
| | SYGFQPTNGVGYQPYRVVVLSFELLHAPATVCGPKKSTNLVKNKCV |
| | NFNFNGLTGTGVLTESNKKFLPFQQFGRDIADTTDAVRDPQTLEILDIT |
| | PCSFGGVSVITPGTNTSNQVAVLYQDVNCTEVPVAIHADQLTPTWRV |
| | YSTGSNVFQTRAGCLIGAEHVNNSYECDIPIGAGICASYQTQTNSPRR |
| | ARSVASQSIIAYTMSLGAENSVAYSNNSIAIPTNFTISVTTEILPVSMTK |
| | TSVDCTMYICGDSTECSNLLLQYGSFCTQLNRALTGIAVEQDKNTQE |
| | VFAQVKQIYKTPPIKDFGGFNFSQILPDPSKPSKRSFIEDLLFNKVTLAD |
| | AGFIKQYGDCLGDIAARDLICAQKFNGLTVLPPLLTDEMIAQYTSALL |
| | AGTITSGWTFGAGAALQIPFAMQMAYRENGIGVTQNVLYENQKLIAN |
| | QFNSAIGKIQDSLSSTASALGKLQDVVNQNAQALNTLVKQLSSNFGAI |
| | SSVLNDILSRLDKVEAEVQIDRLITGRLQSLQTYVTQQLIRAAEIRASA |
| | NLAATKMSECVLGQSKRVDFCGKGYHLMSFPQSAPHGVVFLHVTYV |
| | PAQEKNFTTAPAICHDGKAHFPREGVFVSNGTHWFVTQRNFYEPQIIT |
| | TDNTFVSGNCDVVIGIVNNTVYDPLQPELDSFKEELDKYFKNHTSPDV |
| | DLGDISGINASVVNIQKEIDRLNEVAKNLNESLIDLQELGKYEQYIKWP |
| | WYIWLGFIAGLIAIVMVTIMLCCMTSCCSCLKGCCSCGSCCKFDEDDS |
| | EPVLKGVKLHYT |

The term variant of SARS-CoV-2 used interchangeably herein with a “heterogeneous SARS-CoV-2 strain” is a SARS-CoV-2 virus comprising a SARS-CoV-2 S glycoprotein (also called “CoV S polypeptide”) having at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, or at least about 35 modifications, from about 1 to about 100 modifications, from about 2 to about 35 modifications, from about 5 to about 10 modifications, from about 5 to about 20 modifications, from about 10 to about 20 modifications, from about 15 to about 25 modifications, from about 20 to about 30 modifications, from about 20 to about 40 modifications, from about 25 to about 45 modifications, from about 25 to about 50 modifications, from about 25 to about 55 modifications, from about 30 to about 60 modifications, from about 25 to about 100 modifications, from about 25 to about 45 modifications, or from about 35 to about 100 modifications as compared to a CoV S polypeptide having the amino acid sequence of SEQ ID NO: 1 or SEQ ID NO: 2.

In embodiments, the heterogeneous SARS-CoV-2 strain is a SARS-CoV-2 virus comprising a CoV S polypeptide with at least about 70%, at least 71%, at least 72%, at least 73%, at least 74%, at least about 75%, at least 76%, at least 77%, at least 78%, at least 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 70% and about 99.9% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 70% and about 99.5% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 90% and about 99.9% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 90% and about 99.8% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 95% and about 99.9% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 95% and about 99.8% identity to a CoV S polypeptide having the amino acid sequence of SEQ ID NO: 1 or SEQ ID NO: 2. In embodiments, the heterogeneous SARS-CoV-2 strain is a SARS-CoV-2 virus comprising a CoV S polypeptide with between about 95% and about 99% identity to a CoV S polypeptide having the amino acid sequence of SEQ ID NO: 1 or SEQ ID NO: 2.

In embodiments, the heterogeneous SARS-CoV-2 strain has a World Health Organization Label of alpha, beta, gamma, delta, epsilon, eta, iota, kappa, zeta, mu, or omicron. In embodiments, the heterogeneous SARS-CoV-2 strain has a PANGO lineage selected from the group consisting of B.1.1.529; BA.1, BA.1.1, BA.2, BA.3, BA.4, BA.5, B.1.1.7, B. 1.351,P.1, B.1.617.2, AY, B.1.427, B.1.429, B.1.525, B.1.526, B. 1.617.1, B. 1.617.3, P.2, B. 1.621, or B.1.621.1. The following document describes the Pango lineage designation and is incorporated by reference herein in its entirety: O′Toole et al. BMC Genomics, 23, 121 (2022).

In embodiments, the heterogeneous SARS-CoV-2 strain has a World Health Organization Label of omicron. In embodiments, the heterogeneous SARS-CoV-2 strain with a World Health Organization Label of omicron has at least 35 modifications compared to the wild-type SARS-CoV-2 S polypeptide of SEQ ID NO: 2. In embodiments, the heterogeneous SARS-CoV-2 strain with a World Health Organization Label of omicron has from 35 to 55, from 35 to 65, from 35 to 75, from 35 to 85, from 35 to 95, or from 35 to 105 modifications compared to the wild-type SARS-CoV-2 S polypeptide of SEQ ID NO: 2. In embodiments, the modifications are selected from the group consisting of T6I, T6R, A14S, A54V, V70A, T82I, G129D, H133Q, K134E, W139R, E143G, F144L, Q170E, 1197V, L199I, V200E, V200G, G239V, G244S, G326D, G326H, R333T, L355I, S358F, S358L, S360P, S362F, T363A, D392N, R395S, K404N, N427K, K431T, V432P, G433S, L439R, L439Q, N447K, S464N, T465K, E471A, F473V, F473S, F477S, Q480R, G483S, Q485R, N488Y, Y492H, T534K, T591I, D601G, G626V, H642Y, N645S, N666K, P668H, S691L, N751K, D783Y, N843K, Q941H, N956K, L968F, D1186N, deletion of amino acid 11, deletion of amino acid 12, deletion of amino acid 13, deletion of amino acid 56, deletion of amino acid 57, deletion of amino acid 130, deletion of amino acid 131, deletion of amino acid 132, deletion of amino acid 144, deletion of amino acid 145, deletion of amino acid 198, and insertion of a tripeptide having the amino acid sequence of EPE between amino acids 214 and 215, and combinations thereof

In embodiments, the CoV S polypeptide of the variant comprises a combination of modifications selected from the group consisting of:
(i) A54V, T82I, G129D, L199I, G326D, S358L, S360P, S362F, K404N, N427K, G433S, S464N, T465K, E471A, Q480R, G483S, Q485R, N488Y, Y492H, T534K, D601G, H642Y, N666K, P668H, N751K, D783Y, N843K, Q941H, N956K, L968F, deletion of amino acid 56, deletion of amino acid 57, deletion of amino acid 130, deletion of amino acid 131, deletion of amino acid 132, deletion of amino acid 198, and insertion of a tripeptide having the amino acid sequence of EPE between amino acids 214 and 215;
(ii) T6I, A14S, G129D, V200G, G326D, S358F, S360P, S362F, T363A, D392N, R395S, K404N, N427K, S464N, T465K, E471A, Q480R, Q485R, N488Y, Y492H, D601G, H642Y, N666K, P668H, N751K, D783Y, Q941H, N956K, deletion of amino acid 11, deletion of amino acid 12, and deletion of amino acid 13;
(iii) T6R, A14S, T82I, G129D, E143G, L199I, G326D, S358L, S360P, K404N, N427K, G433S, S464N, T465K, E471A, Q480R, G483S, Q485R, N488Y, Y492H, T534K, D601G, H642Y, N666K, P668H, N751K, D783Y, N843K, Q941H, N956K, L968F, deletion of amino acid 144, deletion of amino acid 145, deletion of amino acid 198, and insertion of a tripeptide having the amino acid sequence of EPE between amino acids 214 and 215;
(iv) T6I, A14S, G129D, V200G, G326D, S358F, S360P, S362F, T363A, D392N, K404N, N427K, L439Q, S464N, T465K, E471A, Q480R, Q485R, N488Y, Y492H, D601G, H642Y, N666K, P668H, S691L, N751K, D783Y, Q941H, N956K, deletion of amino acid 11, deletion of amino acid 12, and deletion of amino acid 13;
(v) T6I, A14S, G129D, V200G, G326D, S358F, S360P, S362F, T363A, D392N, S464N, T465K, E471A, Q480R, Q485R, N488Y, Y492H, D601G, H642Y, N666K, P668H, D783Y, Q941H, N956K, deletion of amino acid 11, deletion of amino acid 12, and deletion of amino acid 13;
(vi) T6I, A14S, G129D, V200G, G326D, S358F, S360P, S362F, T363A, D392N, R395S, K404N, D601G, H642Y, N645S, N666K, N751K, D783Y, Q941H, N956K, deletion of amino acid 11, deletion of amino acid 12, deletion of amino acid 13, deletion of amino acid 56, and deletion of amino acid 57;
(vii) V3G, T6I, A14S, G129D, V200G, G326D, S358F, S360P, S362F, T363A, D392N, R395S, K404N, L439R, S464N, T465K, E471A, F473V, Q485R, N488Y, Y492H, D601G, G626V, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(viii) V3G, T6I, A14S, G129D, V200G, G326D, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(ix) T6I, A14S, G129D, V200G, G326D, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(x) T6I, A14S, G129D, K134E, W139R, F144L, 1197V, V200G, G244S, G326H, S358F, S360P, S362F, T363A, D392N, R395S, K404N, N427K, G433S, N447K, S464N, T465K, E471A, Q485R, N488Y, Y492H, D601G, H642Y, N666K, P668H, N751K, D783Y, Q941H, N956K, deletion of amino acid 11, deletion of amino acid 12, and deletion of amino acid 13;
(xi) T6I, A14S, G129D, K134E, W139R, F144L, I197V, V200G, G244S, G326H, R333T, S358F, S360P, S362F, T363A, D392N, R395S, K404N, N427K, G433S, L439R, N447K, S464N, T465K, E471A, F473S, Q485R, N488Y, Y492H, T5911, D601G, H642Y, N666K, P668H, N751K, D783Y, Q941H, N956K, D1186N, deletion of amino acid 11, deletion of amino acid 12, and deletion of amino acid 13;
(xii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N645S, N666K, P668H, N751K, D783Y, Q941H, N956K, deletion of amino acid 11, deletion of amino acid 12, deletion of amino acid 13, deletion of amino acid 56, and deletion of amino acid 57;
(xiii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(xiv) T6I, A14S, V70A, G129D, H133Q, Q170E, V200E, G239V, G326H, R333T, L355I, S358F, S360P, S362F, T363A, D392N, R395S, K404N, N427K, V432P, G433S, N447K, S464N, T465K, E471A, F473S, F477S, Q485R, N488Y, Y492H, D601G, H642Y, N666K, P668H, N751K, D783Y, Q941H, N956K, deletion of amino acid 11, deletion of amino acid 12, deletion of amino acid 13, and deletion of amino acid 131;
(xv) T6I, A14S, G129D, H133Q, Q170E, V200E, G326H, R333T, L355I, S358F, S360P, S362F, T363A, D392N, R395S, K404N, N427K, V432P, G433S, N447K, S464N, T465K, E471A, F473S, F477S, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, deletion of amino acid 57, and deletion of amino acid 131;
(xvi) T6I, A14S, G129D, V200G, G326D, R333T, S358F, S360P, S362F, T363A, D392N, R395S, K404N, N427K, K431T, L439R, N447K, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57;
(xvii) T6I, A14S, G129D, V200G, G326D, S358F, S360P, S362F, T363A, D392N, R395S, K404N, N427K, K431T, L439R, N447K, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, and deletion of amino acid 57; and
(xviii) T6I, A14S, G129D, V200G, G326D, R333T, S358F, S360P, S362F, T363A, D392N, R395S, K404N, N427K, L439R, S464N, T465K, E471A, F473V, Q485R, N488Y, Y492H, D601G, H642Y, N666K, P668H, N751K, D783Y, Q941H, N956K, deletion of amino acid 11, deletion of amino acid 12, deletion of amino acid 13, deletion of amino acid 56, deletion of amino acid 57, and deletion of amino acid 131;
(xix) deletion of amino acid 56, deletion of amino acid 57, and deletion of amino acid 131, N488Y, A557D, D601G, P668H or P668R, T703I, S969A, and D1105H;
(xx) D67A, K404N, E471K, N488Y, D601G, and A688V;
(xxi) D67A, D202G, L229H, K404N, E471K, N488Y, D601G, and A688V;
(xxii) D67A, D202G, deletion of 1, 2, or 3 amino acids of amino acids 228-230, K404N, E471K, N488Y, D601G, and A688V;
(xxiii) D67A, L229H, R2331, N488Y, K404N, E471K, D601G, and A688V; (xxiv) L5F, T7N, P13S, D125Y, R177S, K404T, E471K, N488Y, D601G, H642Y, T1014I, and V1163F;
(xxv) W139C and L439;
(xxvi) deletion of amino acid 144, deletion of amino acid 145, T6R, E143G, L439R, T465K, D601G, P668R, and D937N;
(xxvii) deletion of amino acid 144, deletion of amino acid 145, T6R, G129D, E143G, L439R, T465K, D601G, P668R, and D937N;
(xxviii) deletion of amino acid 144, deletion of amino acid 145, T6R, T82I, G129D, Y132H, E143G, A209V, K404N L439R, T465K, D601G, P668R, and D937N;
(xxix) deletion of amino acid 144, deletion of amino acid 145, T6R, G129D, E143G, W245I, K404N, N426K, L439R, T465K, E471K, N488Y, D601G, P668R, and D937N;
(xxx) deletion of amino acid 144, deletion of amino acid 145, T6R, W51H, H53W, G129D, E143G, D200V, L201R, W245I, K404N, N426K, L439R, T465K, E471K, N488Y, D601G, P668R, and D937N;
(xxxi) deletion of amino acid 144, deletion of amino acid 145, T6R, G129D, E143G, K404N, L439R, T465K, E471Q, D601G, P668R, and D937N;
(xxxii) Q39R, A54V, E471K; D601G, Q664H, F875L, and deletion of 1, 2, 3, or 4 of amino acids 56, 57, 131, 132;
(xxxiii) T82I, D240G, E471K, D601G, and A688V;
(xxxiv) L439R, E471Q, D601G, P668R, and Q1058H;
(xxxv) G62V, T63I, R233N, L439Q, F477S, D601G, T846N, and deletion of 1, 2, 3, 4, 5, or 6 of amino acids 234-240;
(xxxvi) T821, Y131S, Y132N, R333K, E471K, N488Y, D601G, P668H, and D937N; and
(xxxvii) G129D, G326D, S360P, S362F, K404N, N427K, T465K, E471A or E471K, Q480K or Q480R, Q485R, N488Y, Y492H, D601G, H642Y, N666K, P668H, N751K, D783Y, Q941H, and N953K;
wherein the amino acids of the CoV S glycoprotein are numbered with respect to a polypeptide having the sequence of SEQ ID NO: 2.

As used herein, the term “NVX-CoV2373” refers to a vaccine composition comprising the BV2373 Spike glycoprotein (SEQ ID NO: 3) and Fraction A and Fraction C iscom matrix (e.g., MATRIX-MIM).

The term “pseudovirus” refers to a recombinant viral particle that encodes one or more proteins from a first virus and an envelope protein from a second virus, wherein the first virus and second virus are different. In embodiments, the proteins from a first virus are selected from one or more of Gag, Pol, Rev, and Tat. In embodiments, the envelope protein is a spike protein. In embodiments, the envelope protein is a Spike protein from a SARS-CoV-2 S virus or a variant thereof. The following reference describes pseudoviruses and is incorporated by reference herein in its entirety: Li et al. Rev Med Virol. 2018 January; 28(1): e1963.

The term “transfer plasmid” refers to a nucleic acid plasmid comprising an insertion point for a gene of interest (GOI) and nucleic acids that facilitate packaging and insertion of the GOI into a host cell genome. In embodiments, the GOI encodes a reporter protein.

### Assays for Evaluating the Immunogenicity of Vaccine Compositions Against SARS-CoV-2

The disclosure provides pseudoviruses and methods of using pseudoviruses to evaluate the immunogenicity of immunogenic compositions and vaccine compositions against SARS-CoV-2 or variants thereof. The pseudoviruses are also used to determine if a biological sample contains antibodies that neutralize SARS-CoV-2 or variants thereof.

### Pseudoviruses

In embodiments, provided herein are pseudoviruses encoding: (i) a SARS-CoV-2 Spike (S) glycoprotein; and (ii) one or more of Rev, Tat, Gag, and Pol proteins. In embodiments, the pseudoviruses further encode a reporter protein.

In embodiments, the pseudoviruses encode from 1 to 30, from 1 to 20, from 2 to 20, from 3 to 20, from 4 to 20, from 5 to 20, from 6 to 20, from 7 to 20, from 8 to 20, from 9 to 20, from 10 to 20, from 11 to 20, from 12 to 20, from 13 to 20, from 14 to 20, from 15 to 20, from 16 to 20, from 17 to 20, from 18 to 20, from 19 to 20, from 1 to 10, from 2 to 10, from 3 to 10, from 4 to 10, from 5 to 10, from 6 to 10, from 7 to 10, from 8 to 10, from 2 to 5, from 3 to 5, from 4 to 8, from 5 to 8, or from 6 to 8 SARS-CoV-2 S glycoproteins. In embodiments, the pseudoviruses encode 1, 2, 3, 4, 5, 6, 7, 8, 9, about 10, about 11, about 12, about 13, about 14, about 15, about 16, about 17, about 18, about 19, about 20, about 21, about 22, about 23, about 24, about 25, about 26, about 27, about 28, about 29, or about 30 SARS-CoV-2 S glycoproteins.

Suitable SARS-CoV-2 S glycoproteins include the SARS-CoV-2 S glycoproteins associated with Protein Data Bank (PDB) IDs of any one of: 6LVN,6LZG,6MOJ,6M17,6M1V,6VSB,6VW1,6VXX,6VYB,6W41,6WPS,6WPT,6X29,6X2A,6X2B,6X2C,6X45,6X6P,6X79,6XC2,6XC3,6XC4,6XC7,6XCM,6XCN,6XDG,6XE1,6XE Y,6XF5,6XF6,6XKL,6XKP,6XKQ,6XLU,6XM0,6XM3,6XM4,6XM5,6XR8,6XRA,6XS6,6YBB,6YLA,6YM0,6YOR,6YZ5,6YZ7,6Z2M,6Z43,6Z97,6ZB4,6ZB5,6ZBP,6ZCZ,6ZDG,6ZDH,6ZER,6ZFO,6ZGE,6ZGG,6ZGH,6ZGI,6ZH9,6ZHD,6ZLR,6ZOW,6ZOX,6ZOY,6ZOZ, 6ZP0,6ZP1,6ZP2,6ZP5,6ZP7,6ZWV,6ZXN,7A25,7A29,7A4N,7A5R,7A5S,7A91,7A92,7A 93,7A94,7A95,7A96,7A97,7A98,7AD1,7AKD,7BOB,7B14,7B17,7B18, 7B27, 7B30, 7B62, 7 BEH,7BEI, 7BEJ, 7BEK, 7BEL, 7BEM, 7BEN,7BEO,7BEP,7BH9,7BNM,7BNN, 7BNO,7BNV, 7BWJ,7BYR,7BZ5,7C01,7C2L,7C53,7C8D,7C8J,7C8V,7C8W,7CAB,7CAC,7CAH,7CAI,7 CAK,7CAN,7CDI,7CDJ,7CH4,7CH5,7CHB,7CHC,7CHE,7CHF,7CHH,7CHO,7CHP,7CHS, 7CJF,7CM4,7CN9,7COT,7CT5,7CWL,7CWM,7CWN,7CWO,7CWS,7CWT,7CWU,7CYH, 7CYP,7CYV,7CZP,7CZQ,7CZR,7CZS,7CZT,7CZU, 7CZV,7CZW, 7CZX, 7CZY,7CZZ, 7D0 0,7D03,7D0B,7D0C,7D0D,7D2Z,7D30,7D4G,7D6I,7DCC,7DCX,7DD2,7DD8,7DDD,7DD N,7DE0,7DET,7DEU,7DF3,7DF4,7DHX,7DJZ,7DK0,7DK2,7DK3,7DK4,7DK5,7DK6,7D K7,7DMU,7DPM,7DQA,7DTE,7DWX,7DWY,7DWZ,7DX0,7DX1,7DX2, 7DX3, 7DX4, 7D

X5,7DX6,7DX7,7DX8,7DX9,7DZW,7DZX,7DZY,7E23,7E39,7E3B,7E3C,7E3J,7E3K,7E3 L,7E30,7E50,7E5R,7E5S,7E5Y,7E7B,7E7D,7E7X,7E7Y,7E86,7E88,7E8C,7E8F,7E8M,7E 9N,7E90,7E9P,7E9Q,7E9T,7EAM,7EAN,7EAZ,7EB0,7EB3,7EB4,7EB5,7EDF,7EDG,7ED H,7EDI,7EDJ,7EFP,7EFR,7EH5,7EJ4,7EJ5,7EJL,7EJY,7EJZ,7EK0,7EK6,7EKC,7EKE,7E KF,7EKG,7EKH,7ENF,7ENG,7EPX,7EY0,7EY4,7EY5,7EYA,7EZV,7FOX,7F12,7F15,7F3 Q,7F46,7F5H,7F5R,7F62,7F63,7F6Y,7F6Z,7F7E,7F7H,7FAE,7FAF,7FAT,7FAU,7FB0,7FB 1,7FB3,7FB4,7FBJ,7FBK,7FC5,7FCD,7FCE,7FCP,7FCQ,7FDG,7FDH,7FDI,7FDK,7FEM, 7FET,7FG2,7FG3,7FG7,7FH0,7FJC,7FJN,7FJO,7FJS,7JJC,7JJI,7JJJ,7JMO,7JMP,7JMW,7J V2,7JV4,7JV6,7JVA,7JVB,7JVC,7JW0,7JWB,7JWY,7JX3,7JZL,7JZM,7JZN,7JZU,7K43,7 K45,7K4N,7K8M,7K8S,7K8T,7K8U,7K8V,7K8W,7K8X,7K8Y,7K8Z,7K90,7K9H,7K9I,7 K9J,7K9K,7K9Z,7KDG,7KDH,7KDI,7KDJ,7KDK,7KDL,7KE4,7KE6,7KE7,7KE8,7KE9,7 KEA,7KEB,7KEC,7KFV,7KFW,7KFX,7KFY,7KGJ,7KGK,7KJ2,7KJ3,7KJ4,7KJ5,7KKK,7 KKL,7KL9,7KLG,7KLH,7KLW,7KM5,7KMB,7KMG,7KMH,7KMI,7KMK,7KML,7KMS, 7KMZ,7KN3,7KN4,7KN5,7KN6,7KN7,7KNB,7KNE,7KNH,7KNI,7KQE,7KRQ,7KRR,7K RS,7KS9,7KSG,7KXJ,7KXK,7KZB,7L02,7L06,7L09,7L0N,7L2C,7L2D,7L2E,7L2F,7L3N, 7L4Z,7L56, 7L57,7L58,7L5B,7L7D,7L7E,7L7F, 7L7K, 7LAA,7LAB,7LC8,7LCN,7LD1,7LD J,7LJR,7LM8,7LO4,7LOP,7LQ7,7LQV,7LQW,7LRS,7LRT,7LS9,7LSS,7LWI,7LWJ,7LW K,7LWL,7LWM,7LWN,7LWO,7LWP,7LWQ,7LWS,7LWT,7LWU,7LWV,7LWW,7LX5,7 LXW,7LXX,7LXY,7LXZ,7LY0,7LY2,7LY3,7LYK,7LYL,7LYM,7LYN,7LY0,7LYP,7LY Q,7MOJ,7M31, 7M42, 7M53, 7M6D,7M6E,7M6F,7M6G,7M6H,7M61,7M71,7M7B,7M7W,7 M8J,7M8K,7M8S,7M8T,7M8U,7MDW,7ME7,7MEJ,7MF1,7MFU,7MJG,7MJH,7MJI,7MJJ, 7MJK,7MJL,7MJM,7MJN,7MKL,7MKM,7MLZ,7MM0,7MMO,7MSQ,7MTC,7MTD,7MT E,7MW2,7MW3,7MW4,7MW5,7MW6,7MY2,7MY3,7MY8,7MZF,7MZG,7MZH,7MZI,7M ZJ,7MZK,7MZL,7MZM,7MZN,7NOG,7NOH,7N1A,7N1B,7NIE,7NIF,7NIQ,7NIT,7NIU,7 NIV,7NIW,7NIX,7NIY,7N31,7N41,7N4J,7N4L,7N4M,7N5H,7N62,7N64,7N6D,7N6E,7N8 H,7N81,7N9A,7N9B,7N9C,7N9E,7N9T,7NAB,7ND3,7ND4,7ND5,7ND6,7ND7,7ND8,7ND 9,7NDA,7NDB,7NDC,7NDD,7NEG,7NEH,7NKT,7NLL,7NP1, 7NS6,7NT9,7NTA,7NTC,7 NX6,7NX7,7NX8,7NX9,7NXA,7NXB,7NXC,70AN,70A0,70AP,70AQ,70AU,70AY,70 D3,7ODL,7OLZ,7OR9,7ORA,7ORB,70WX,7P19,7P3D,7P40,7P5G,7P5Q,7P5S,7P77,7P78, 7P79,7P7A,7P7B,7PBE,7PHG,7PQY,7PQZ,7PR0,7PRY,7PRZ,7PS0,7PS1,7PS2,7PS4,7PS5, 7PS6,7PS7,7Q0A,7Q0G,7Q0H,7Q01,7Q1Z,7Q3Q,7Q3R,7Q6E,7Q9F,7Q9G,7Q91,7Q9J,7Q9 K,7Q9M,7Q9P,7QEZ,7QF0,7QF1,7QNW,7QNX,7QNY,7Q07,7Q09,7QTI,7QTJ,7QTK,7Q UR,7QUS,7ROZ,7R10,7R11,7R12,7R13,7R14,7R15,7R16,7R17,7R18,7R19,7R1A,7RIB,7R 40,7R4I,7R4Q,7R4R,7R6W,7R6X,7R7N,7R8L,7R8M,7R8N,7R80,7R95,7RA8,7RAL,7RA Q,7RBU,7RBV,7RBY,7RKU,7RKV,7RNJ,7RPV,7RQ6,7RR0,7RTD,7RTR,7RU1,7RU2,7R U3,7RU4,7RU5,7RU8,7RW2,7RXD,7RZQ,7RZR,7RZS,7RZT,7RZU,7RZV,7SOB,7SOC,7S 0D,7SOE,7S3N,7S4S,7S5P,7S5Q,7S5R,7S61,7S6J,7S6K,7S6L,7S83,7SA2,7SBK,7SBL,7SB 0,7SBP,7SBQ,7SBR,7SBS,7SBT,7SBU,7SC1,7SD5,7SI2,7SIS,7SIX,7SJ0,7SJS,7SKZ,7SL5, 7SN0,7SN2,7SN3,7SO9,7SOA,7SOB,7SOC,7SOD,7SOE,7SOF,7SPO,7SPP,7SWN,7SWO, 7SWP,7SWW,7SWX,7SXR,7SXS,7SXT,7SXU,7SXV,7SXW,7SXX,7SXY,7SXZ,7SY0,7S Y1,7SY2,7SY3,7SY4,7SY5,7SY6,7SY7,7SY8,7T01,7T3M,7T67,7T72,7T7B,7T9J,7T9K,7T 9L,7TAS,7TAT,7TB4,7TB8,7TBF,7TCA,7TCC,7TCQ,7TEI,7TEW,7TEX,7TEY,7TEZ,7TF 0,7TF1,7TF2,7TF3,7TF4,7TF5,7TF8,7TGE,7TGW,7TGX,7TGY,7THE,7THK,7THT,7TIK, 7TL1,7TL9,7TLA,7TLB,7TLC,7TLD,7TLT,7TLY,7TM0,7TN0,7TNW,7TO4,7TOU,7TOV, 7TOW,7TOX,7TOY,7TOZ,7TP0,7TP1,7TP2,7TP3,7TP4,7TP7,7TP8,7TP9,7TPA,7TPC,7TP E,7TPF,7TPH,7TPK,7TPL,7TPR,7TYZ,7TZ0,7U09,7UOA,7UOD,7UOE,7UON,7UOP,7UOQ, 7U0X,7U1R,7U2D,7U2E,7U8E,7U9O,7U9P,7UAP,7UAQ,7UAR,7UB0,7UB5,7UB6,7UFK, 7UFL,7UHC,7UL0,7UM2,7UPL,7UR1,7URQ,7URS,7UZ4,7UZ5,7UZ6,7UZ7,7UZ8,7UZ9, 7UZA,7UZB,7UZC,7UZD,7V20,7V22,7V23,7V24,7V26,7V27,7V2A,7V76,7V77,7V78,7V 79,7V7A,7V7D,7V7E,7V7F,7V7G,7V7H,7V71,7V7J,7V7N,7V7O,7V7P,7V7Q,7V7R,7V7S, 7V7T,7V7U,7V7V,7V7Z,7V80,7V81,7V82,7V83,7V84,7V85,7V86,7V87,7V88,7V89,7V8 A,7V8B,7V8C,7VHH,7VHJ,7VHK,7VHL,7VHM,7VHN,7VMU,7VNB,7VNC,7VND,7VN E,7VOA,7VQ0,7VRV,7VRW,7VX1,7VX4,7VX5,7VX9,7VXA,7VXB,7VXC,7VXD,7VXE, 7VXF,7VXI,7VXK,7VXM,7VYR,7VZT,7W1S,7W6U,7W8S,7W92,7W94,7W99,7W9B,7 W9C,7W9E,7W9F,7WA1,7WB5,7WBL,7WBP,7WBQ,7WBZ,7WCD,7WCH,7WCK,7WCP, 7WCR,7WCU,7WCZ,7WD0,7WD1,7WD2,7WD7,7WD8,7WD9,7WDF,7WE7,7WE8,7WE 9,7WEA,7WEB,7WEC,7WED,7WEE,7WEF,7WEV,7WG6,7WG7,7WG8,7WG9,7WGB,7 WGC,7WGV,7WGX,7WGY,7WGZ,7WH8,7WHB,7WHD,7WHH,7WHI,7WHJ,7WHK,7W HZ,7WJY,7WJZ,7WK0,7WK2,7WK3,7WK4,7WK5,7WK6,7WK8,7WK9,7WKA,7WLC,7 WM0,7WN2,7WNB,7WNM,7WO4,7WO5,7WO7,7WOA,7WOB,7WOC,7WOG,7WON,7 WOP,7WOQ,7WOR,7WOS,7WOU,7WOV,7WOW,7WP0,7WP1,7WP2,7WP5,7WP6,7WP8, 7WP9,7WPA,7WPB,7WPC,7WPD,7WPE,7WPF,7WPH,7WQV,7WR8,7WRH,7WRI,7WR J,7WRV,7WS0,7WS1,7WS2,7WS3,7WS4,7WS5,7WS6,7WS7,7WS8,7WS9,7WSA,7WSE,7 WSH,7WSK,7WT7,7WT8,7WT9,7WTF,7WTG,7WTH,7WTI,7WTJ,7WTK,7WUE,7WUH, 7WVL,7WVP,7WVQ,7WWI,7WWJ,7WWK,7WWL,7WWM,7WXZ,7WZ1,7WZ2,7X1M,7 X25,7X2H,7X2K,7X2L,7X2M,7X63,7X66,7X6A,7X7D,7X7E,7X7N,7X7T,7X7U,7X8W,7 X8Y,7X8Z,7X90,7X91,7X92,7X93,7X94,7X95,7X96,7X9E,7XA7,7XAZ,7XB0,7XB1,7XB Y,7XCH,7XCI,7XCK,7XCO,7XCP,7XCZ,7XD2,7XDA,7XDB,7XDK,7XDL,7XEG,7XEI,7 XH8,7XIC,7XID,7XIK,7XIL,7XIW,7XIX,7XIY,7XIZ,7XJ6,7XJ8,7XJ9,7XMX,7XMZ,7XN Q,7XNR,7XNS,7XO4,7XO5,7XO6,7XO7,7XO8,7XO9,7XOA,7XOB,7XOC,7XOD,7XRP,7 XS8,7XSA,7XSB,7XSC,7XST,7XTZ,7XU0,7XU1,7XU2,7XU3,7XU4,7XU5,7XU6,7XWA, 7XXL,7Y0C,7Y0V,7Y1Y,7Y1Z,7Y42,7Y6D,7Y6K,7Y6L,7Y6N,7Y75,7Y76,7Y7J,7Y7K,7 Y8J,7Y9N,7Y9S,7Y9Z,7YA0,7YA1,7YAD,7YBI,7YBJ,7YC5,7YCK,7YCL,7YCN,7YCO,7 YD1,7YDI,7YDY,7YE5,7YE9,7YEG,7YH6,7YH7,7YHW,7YJ3,7YKJ,7YOW,7YQT,7YQ U,7YQV,7YQW,7YQX,7YQY,7YQZ,7YR0,7YR1,7YR2,7YR3,7YTN,7YUE,7YV8,7YVE, 7YVF,7YVG,7YVH,7YVI,7YVJ,7YVK,7YVL,7YVM,7YVN,7YVO,7YVP,7YVU,7Z0X,7 Z0Y,7Z1A,7Z1B,7Z1C,7Z1D,7Z1E,7Z3Z,7Z6V,7Z7X,7Z85,7Z86,7Z80,7Z9Q,7Z9R,7ZBU, 7ZCE,7ZCF,7ZDQ,7ZF3,7ZF4,7ZF5,7ZF7,7ZF8,7ZF9,7ZFA,7ZFB,7ZFC,7ZFD,7ZFE,7ZJ L,7ZR2,7ZR7,7ZR8,7ZR9,7ZRC,7ZRV,7ZSD,7ZSS,7ZXU,8A99,8AAA,8AQS,8AQT,8AQ U,8AQV,8AQW,8BBN,8BBO,8BCZ,8BE1,8BEV,8BGG,8BH5,8BON,8BSE,8BSF,8C1V,8 C3V,8C8P,8CIM,8CSA,8CSJ,8CWI,8CWK,8CWU,8CWV,8CXN,8CXQ,8CY6,8CY7,8CY 9,8CYA,8CYB,8CYC,8CYD,8CYJ,8CZI,8D0Z,8D36,8D47,8D48,8D55,8D56,8D5A,8D6Z, 8D8Q,8D8R,8DAD,8DAO,8DCC,8DCE,8DF5,8DGU,8DI5,8DLI,8DLJ,8DLK,8DLL,8DLM, 8DLN,8DLO,8DLP,8DLQ,8DLR,8DLS,8DLT,8DLU,8DLV,8DLW,8DLX,8DLY,8DLZ,8D M0,8DM1,8DM2,8DM3,8DM4,8DM5,8DM6,8DM7,8DM8,8DM9,8DMA,8DNN,8DT3,8D T8,8DTR,8DTT,8DTX,8DV1,8DV2,8DW2,8DW3,8DW9,8DWA,8DXS,8DXT,8DXU,8DY A,8DZH,8DZI,8E1G,8EL2,8ELH,8ELJ,8ELO,8ELP,8ELQ,8EOO,8EPN,8EPP,8EPQ,8ERQ, 8ERR,8FOG,8FOH,8FA1,8FA2,8FEZ,8FU7,8FU8,8FU9,8GB0,8GB5,8GB6,8GB7,8GB8,8G JM,8GJN,8GOM,8GON,8GOU,8GPY,8GRY,8GS6,8GS9,8GTO,8GTP,8GTQ,8GX9,8GZ5, 8GZZ,8H00,8H01,8H06,8H07,8H08,8H3D,8H3E,8H3M,8H3N,8H5C,8HC2,8HC3,8HC4,8 HC5,8HC6,8HC7,8HC8,8HC9,8HCA,8HCB,8HEB,8HEC,8HED,8HHX,8HHY,8HHZ,8HN 6,8HN7,8I5H,8I5I,8IDN,8IF2,8IOS,8IOT,8IOU,8IOV,8ITU,8JIQ,8J26,8SMT. Each of these PDB IDs is incorporated by reference herein in its entirety. The amino acid sequence of the SARS-CoV-2 S glycoprotein associated with each entry may be accessed by downloading the FASTA file associated with the PDB ID at www.rcsb.org. In embodiments, the pseudoviruses express a nucleic acid encoding a SARS-CoV-2 S glycoprotein of any one of the aforementioned PDB IDs.

In embodiments, the SARS-CoV-2 S glycoprotein is a wild-type SARS-CoV-2 S glycoprotein, or a SARS-CoV-2 S glycoprotein from a SARS-CoV-2 variant thereof. In embodiments, the variant of SARS-CoV-2 is a B.1.1.7 SARS-CoV-2 strain; a B.1.351 SARS-CoV-2 strain; a P.1 SARS-CoV-2 strain; a Cal.20C SARS-CoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; a C.37 SARS-CoV-2 strain; a B.1.621 SARS-CoV-2 strain; or a B.1.1.529 SARS-CoV-2 strain.. The wild-type SARS-CoV-2 S glycoprotein contains a furin cleavage site, RRAR (SEQ ID NO: 6) at positions 669-672 of the SARS-CoV-2 S glycoprotein of SEQ ID NO: 2. In embodiments, the SARS-CoV-2 S glycoprotein has an inactive furin cleavage site. In embodiments, the inactive furin cleavage site has the amino acid sequence of any one of SEQ ID NOS: 68-97. In embodiments, the amino acid sequence of the inactive furin cleavage site is GG. In embodiments, non-limiting examples of SARS-CoV-2 S glycoproteins with an inactive furin cleavage site of GG include glycoproteins of SEQ ID NOS: 26-28 and 30.

In embodiments, the amino acid sequence of the inactive furin cleavage site is QQAQ (SEQ ID NO: 68). In embodiments, non-limiting examples of SARS-CoV-2 S glycoproteins with an inactive furin cleavage site of QQAQ (SEQ ID NO: 68) include glycoproteins of SEQ ID NOS: 3, 5-13, 15, 17-19, 21, 23-25, 29, and 31-66.

In embodiments, one or more of the amino acids comprising the native furin cleavage site is mutated to any natural amino acid. In embodiments, one or more of the amino acids comprising the native furin cleavage site is deleted.

In embodiments, one or more of the amino acids comprising the native furin cleavage site is mutated to glutamine. In embodiments, 1, 2, 3, or 4 amino acids may be mutated to glutamine. In embodiments, one of the arginines comprising the native furin cleavage site is mutated to glutamine. In embodiments, two of the arginines comprising the native furin cleavage site are mutated to glutamine. In embodiments, three of the arginines comprising the native furin cleavage site are mutated to glutamine.

In embodiments, one or more of the amino acids comprising the native furin cleavage site, is mutated to alanine. In embodiments, 1, 2, 3, or 4 amino acids may be mutated to alanine. embodiments, one of the arginines comprising the native furin cleavage site is mutated to alanine. In embodiments, two of the arginines comprising the native furin cleavage site are mutated to alanine. In embodiments, three of the arginines comprising the native furin cleavage site are mutated to alanine.

In embodiments, one or more of the amino acids comprising the native furin cleavage site is mutated to glycine. In embodiments, 1, 2, 3, or 4 amino acids may be mutated to glycine. In embodiments, one of the arginines of the native furin cleavage site is mutated to glycine. In embodiments, two of the arginines comprising the native furin cleavage site are mutated to glycine. In embodiments, three of the arginines comprising the native furin cleavage site are mutated to glycine.

In embodiments, one or more of the amino acids comprising the native furin cleavage site, is mutated to asparagine. For example 1, 2, 3, or 4 amino acids may be mutated to asparagine. In embodiments, one of the arginines comprising the native furin cleavage site is mutated to asparagine. In embodiments, two of the arginines comprising the native furin cleavage site are mutated to asparagine. In embodiments, three of the arginines comprising the native furin cleavage site are mutated to asparagine.

In embodiments, the active furin cleavage site (SEQ ID NO: 67) of the SARS-CoV-2 S glycoproteins described herein is replaced with an inactivated furin cleavage site of the table below.

### Inactivated Furin Cleavage Sites

| Amino Acid Sequence of | Active or Inactive |
|---|---|
| Furin Cleavage Site | Furin Cleavage Site |
| RRAR (SEQ ID NO: 67) | Active |
| QQAQ (SEQ ID NO: 68) | Inactive |
| QRAR (SEQ ID NO: 69) | Inactive |
| RQAR (SEQ ID NO: 70) | Inactive |
| RRAQ (SEQ ID NO: 71) | Inactive |
| QQAR (SEQ ID NO: 72) | Inactive |
| RQAQ (SEQ ID NO: 73) | Inactive |
| QRAQ (SEQ ID NO: 74) | Inactive |
| NNAN (SEQ ID NO: 75) | Inactive |
| NRAR (SEQ ID NO: 76) | Inactive |
| RNAR (SEQ ID NO: 77) | Inactive |
| RRAN (SEQ ID NO: 78) | Inactive |
| NNAR (SEQ ID NO: 79) | Inactive |
| RNAN (SEQ ID NO: 80) | Inactive |
| NRAN (SEQ ID NO: 81) | Inactive |
| AAAA (SEQ ID NO: 82) | Inactive |
| ARAR (SEQ ID NO: 83) | Inactive |
| RAAR (SEQ ID NO: 84) | Inactive |
| RRAA (SEQ ID NO: 85) | Inactive |
| AAAR (SEQ ID NO: 86) | Inactive |
| RAAA (SEQ ID NO: 87) | Inactive |
| ARAA (SEQ ID NO: 88) | Inactive |
| GGAG (SEQ ID NO: 89) | Inactive |
| GRAR (SEQ ID NO: 90) | Inactive |
| RGAR (SEQ ID NO: 91) | Inactive |
| RRAG (SEQ ID NO: 92) | Inactive |
| GGAR (SEQ ID NO: 93) | Inactive |
| RGAG (SEQ ID NO: 94) | Inactive |
| GRAG (SEQ ID NO: 95) | Inactive |
| GSAS (SEQ ID NO: 96) | Inactive |
| GSGA (SEQ ID NO: 97) | Inactive |

In embodiments, the SARS-CoV-2 S glycoproteins contain a mutation at Lys-973 of the native SARS-CoV-2 S glycoprotein (SEQ ID NO: 2). In embodiments, Lys-973 is mutated to any natural amino acid. In embodiments, Lys-973 is mutated to proline. In embodiments, Lys-973 is mutated to glycine.

In embodiments, the SARS-CoV-2 S glycoproteins contain a mutation at Val-974 of the native SARS-CoV-2 S glycoprotein (SEQ ID NO: 2). In embodiments, Val-974 is mutated to any natural amino acid, as compared to the SARS-CoV-2 S glycoprotein of SEQ ID NO: 2. In embodiments, Val-974 is mutated to proline. In embodiments, Val-974 is mutated to glycine, as compared to the SARS-CoV-2 S glycoprotein of SEQ ID NO: 2.

In embodiments, the SARS-CoV-2 S glycoproteins contain a mutation at Lys-973 and Val-974 of the native CoV Spike (S) polypeptide (SEQ ID NO: 2). In embodiments, Lys-973 and Val-974 are mutated to any natural amino acid, as compared to the SARS-CoV-2 S glycoprotein of SEQ ID NO: 2. In embodiments, Lys-973 and Val-974 are mutated to proline, as compared to the SARS-CoV-2 S glycoprotein of SEQ ID NO: 2. Non-limiting examples of SARS-CoV-2 S glycoproteins where amino acids Lys-973 and Val-974 are mutated to proline include glycoproteins of SEQ ID NOS: 3-13, 20, and 22-60.

In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 1, SEQ ID NO: 2, or SEQ ID NO: 3.

In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3-66.

In embodiments, the pseudoviruses encode Gag protein and Pol protein. In embodiments, the pseudoviruses encode a fusion protein encoding a Gag protein and Pol protein. In embodiments, the pseudoviruses encode Rev protein and Tat protein. In embodiments, the Gag protein and Pol protein are from a Murine Leukemia Virus (MLV). In embodiments, one or more of the Rev, Tat, Gag, and Pol proteins are from a lentivirus.

In embodiments, the pseudoviruses encode one or more reporter proteins. In embodiments, the pseudoviruses encode from 1 to 10 reporter proteins. In embodiments, the one or more reporter proteins are fluorescent proteins. In embodiments, the fluorescent protein is green fluorescent protein, yellow fluorescent protein, red fluorescent protein, cyan fluorescent protein, enhanced green fluorescent protein, and ZsGreen. In embodiments, the one or more reporter proteins are tagged with a fluorescent dye. In embodiments, the one or more reporter proteins is luciferase.

### Methods of Producing Pseudoviruses

In embodiments, provided herein is a method of producing the pseudovirus described herein, comprising transfecting cells with: (i) a transfer plasmid encoding a reporter protein; (ii) one or more plasmids encoding a Rev protein, a Tat protein, a Gag protein, a Pol protein; or a combination thereof; and (iii) a plasmid expressing a SARS-CoV-2 Spike (S) glycoprotein. In embodiments, the cells are HEK293T (also called “293T”) cells. The SARS-CoV-2 S glycoprotein may be any SARS-CoV-2 S glycoprotein described herein. In embodiments, the SARS-CoV-2 S glycoprotein contains mutation of amino acids 973 and 974 to proline, wherein the SARS-CoV-2 S glycoprotein is numbered according to a SARS-CoV-2 S glycoprotein of SEQ ID NO: 2. In embodiments, the SARS-CoV-2 S glycoprotein contains an inactive furin cleavage site. Numerous examples of inactive furin cleavages sites are described herein.

In embodiments, pseudovirus is produced in a human host cell. In embodiment, pseudovirus is produced in a HEK293T cell (also called “293T” cell).

In embodiments, the plasmid encoding a Rev protein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the plasmid of SEQ ID NO: 100. In embodiments, the plasmid encoding a fusion protein encoding Gag protein and Pol protein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the plasmid of SEQ ID NO: 99. In embodiments, the plasmid encoding a Tat protein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the plasmid of SEQ ID NO: 98.

### Methods of Identifying Neutralizing Antibodies Against SARS-CoV-2 or Variants Thereof

In embodiments, provided herein is a method for determining if a biological sample contains neutralizing antibodies against a SARS-CoV-2 virus or a variant thereof, comprising: (a) contacting a biological sample with from 1 to about 20 pseudoviruses described herein; (b) contacting a cell expressing angiotensin converting enzyme 2 (ACE2) with the from 1 to about 20 pseudoviruses; (c) quantifying expression of the reporter gene in the cell expressing ACE2; (d) contacting a control cell expressing ACE2 with from 1 to about 20 pseudoviruses, wherein the from 1 to about 20 pseudoviruses has not been contacted with the biological sample; and (e) quantifying expression of the reporter gene in the control cell expressing ACE2; wherein if the expression of the reporter gene in the cell of (c) is less than expression of the reporter gene in the control cell of (e), then the biological sample contains neutralizing antibodies against the SARS-CoV-2 virus or the variant thereof.

In embodiments, the method comprises contacting a biological sample with 1, 2, 3, 4, about 5, about 6, about 7, about 8, about 9, about 10, about 11, about 12, about 13, about 14, about 15, about 16, about 17, about 18, about 19, or about 20 pseudoviruses. In embodiments, each pseudovirus expresses a different SARS-CoV-2 S glycoprotein. In embodiments, each pseudovirus expresses a different reporter protein.

In embodiments, the cell expressing ACE2 is produced by transfecting a cell with a plasmid having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the plasmid of SEQ ID NO: 101. In embodiments, ACE2 has an amino acid sequence with having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the polypeptide of SEQ ID NO: 102 or 103.

In embodiments, the biological sample and pseudovirus are incubated from 1 hour to about 24 hours, from 1 hour to about 12 hours, from 1 hour to about 8 hours, from 1 hour to about 6 hours, from 2 to about 6 hours, from 2 to about 7 hours, or from 2 to about 8 hours. In embodiments, the biological sample and pseudovirus are incubated for 1 hour, 2 hours, 3 hours, 4 hours, 5 hours, 6 hours, 7 hours, 8 hours, 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In embodiments, the biological sample and pseudovirus are incubated for 2 or 3 hours. In embodiments, the biological sample and pseudovirus are incubated from 1 day to about 7 days. In embodiments, the biological sample and pseudovirus are incubated for 1 day, 2 days, 3 days, 4 days, 5 days, 6 days, or 7 days. In embodiments, the biological sample and pseudovirus are incubated for three days.

In embodiments, the cell is contacted with the pseudovirus for from 1 hour to about 24 hours. In embodiments, cell is contacted with the pseudovirus are incubated for 1 hour, 2 hours, 3 hours, 4 hours, 5 hours, 6 hours, 7 hours, 8 hours, 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours. In embodiments, the cell is contacted with the pseudovirus for from 1 day to about 7 days. In embodiments, the cell is contacted with the pseudovirus for 1 day, 2 days, 3 days, 4 days, 5 days, 6 days, or 7 days. In embodiments, the cell is contacted with the pseudovirus for three days.

In embodiments, the cell expressing ACE2 is a human cell. In embodiments, the cell expressing ACE2 is a 293T cell. In embodiments, the cell is a A549 cell or a Vero E6 cell. In embodiments, the methods described herein do not require a biosafety level 3 (BSL-3) laboratory.

In embodiments, the cells are selected from the group consisting of: Vero E6, A549, and HEK293T cells. In embodiments, the biological sample is serum. In embodiments, the serum is human serum. In embodiments, the serum is from an individual that has been infected with SARS-CoV-2 or a variant thereof. In embodiments, the serum is from an individual that has received an immunogenic composition or vaccine against SARS-CoV-2 or a variant thereof. In embodiments, prior to use in the methods described herein, the serum, plasma, or blood is heat-inactivated. In embodiments, heat inactivation comprises incubating the serum, plasma, or blood at a temperature of from about 37° C. to about 90° C. In embodiments, heat inactivation comprises incubating the serum, plasma, or blood at a temperature of about 56° C. In embodiments, heat inactivation comprises incubating the serum, plasma, or blood at a temperature of from about 37° C. to about 90° C. for from about 15 minutes to about 2 hours. In embodiments, the serum, plasma, or blood is incubated at about 37° C. to about 90° C. for about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, or about 120 minutes. In embodiments, the serum, plasma, or blood is incubated at about 56° C. for about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, or about 120 minutes. In embodiments, the serum, plasma, or blood is incubated at about 37° C. to about 90° C. for about 30 minutes.

In embodiments, the immunogenic compositions and vaccine compositions contain non-naturally occurring coronavirus (CoV) Spike (S) polypeptides or nanoparticles containing CoV S polypeptides.

In embodiments, the CoV S polypeptide comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1.

In embodiments, the CoV S polypeptide comprises a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2.

In embodiments, the CoV S polypeptide comprises a sequence that is at least 80%, at least 81%, at least 82%, at least 83%, at least 84%, at least 85%, at least 86%, at least 87%, at least 88%, at least 89%, at least 90%, at least 90%, at least 91%, at least 92%, at least 93%, at least 94%, at least 95%, at least 96%, at least 97%, at least 98%, at least 99%, or 100% identical to SEQ ID NO: 3.

The sequence of SEQ ID NO: 3is in the table below.

SEQ ID NO: 3
QCVNLTTRTQLPPAYTNSFTRGVYYPDKVFRSSVLHSTQDLFLPFFSNV
TWFHAIHVSGTNGTKRFDNPVLPFNDGVYFASTEKSNIIRGWIFGTTLD
SKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTP
INLVRDLPQGFSALEPLVDLPIGINITRFQTLLALHRSYLTPGDSSSGW
TAGAAAYYVGYLQPRTFLLKYNENGTITDAVDCALDPLSETKCTLKSFT
VEKGIYQTSNFRVQPTESIVRFPNITNLCPFGEVFNATRFASVYAWNRK
RISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYR
LFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPG
TNTSNQVAVLYQDVNCTEVPVAIHADQLTPTWRVYSTGSNVFQTRAGCL
IGAEHVNNSYECDIPIGAGICASYQTQTNSPQQAQSVASQSIIAYTMSL
GAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDF
GGFNFSQILPDPSKPSKRSFIEDLLFNKVTLADAGFIKQYGDCLGDIAA
RDLICAQKFNGLTVLPPLLTDEMIAQYTSALLAGTITSGWTFGAGAALQ
IPFAMQMAYRFNGIGVTQNVLYENQKLIANQFNSAIGKIQDSLSSTASA
LGKLQDVVNQNAQALNTLVKQLSSNFGAISSVLNDILSRLDPPEAEVQI
DRLITGRLQSLQTYVTQQLIRAAEIRASANLAATKMSECVLGQSKRVDF
CGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEV
AKNLNESLIDLQELGKYEQYIKWPWYIWLGFIAGLIAIVMVTIMLCCMT
SCCSCLKGCCSCGSCCKFDEDDSEPVLKGVKLHYT

In embodiments, the immunogenic compositions or vaccine compositions comprise an adjuvant. Exemplary adjuvants are described throughout this disclosure.

### Aluminum-Based Adjuvants

In embodiments, the adjuvant may be alum (e.g. AlPO₄ or Al(OH)₃). Typically, the nanoparticle is substantially bound to the alum. For example, the nanoparticle may be at least 80% bound, at least 85% bound, at least 90% bound or at least 95% bound to the alum. Often, the nanoparticle is 92% to 97% bound to the alum in a composition. The amount of alum is present per dose is typically in a range between about 400 μg to about 1250 μg. For example, the alum may be present in a per dose amount of about 300 μg to about 900 μg, about 400 μg to about 800 μg, about 500 μg to about 700 μg, about 400 μg to about 600 μg, or about 400 μg to about 500 μg. Typically, the alum is present at about 400 μg for a dose of 120 μg of the protein nanoparticle.

### Saponin Adjuvants

Adjuvants containing saponin may also be combined with the immunogens disclosed herein. Saponins are glycosides derived from the bark of the Quillaja saponaria Molina tree. Typically, saponin is prepared using a multi-step purification process resulting in multiple fractions. As used, herein, the term “a saponin fraction from Quillaja saponaria Molina” is used generically to describe a semi-purified or defined saponin fraction of Quillaja saponaria or a substantially pure fraction thereof.

### Saponin Fractions

Several approaches for producing saponin fractions are suitable. Fractions A, B, and C are described in U.S. Pat. No. 6,352,697 and may be prepared as follows. A lipophilic fraction from Quil A, a crude aqueous Quillaja saponaria Molina extract, is separated by chromatography and eluted with 70% acetonitrile in water to recover the lipophilic fraction. This lipophilic fraction is then separated by semi-preparative HPLC with elution using a gradient of from 25% to 60% acetonitrile in acidic water. The fraction referred to herein as “Fraction A” or “QH-A” is, or corresponds to, the fraction, which is eluted at approximately 39% acetonitrile. The fraction referred to herein as “Fraction B” or “QH-B” is, or corresponds to, the fraction, which is eluted at approximately 47% acetonitrile. The fraction referred to herein as “Fraction C” or “QH-C” is, or corresponds to, the fraction, which is eluted at approximately 49% acetonitrile. Additional information regarding purification of Fractions is found in U.S Pat. No. 5,057,540. When prepared as described herein, Fractions A, B and C of Quillaja saponaria Molina each represent groups or families of chemically closely related molecules with definable properties. The chromatographic conditions under which they are obtained are such that the batch-to-batch reproducibility in terms of elution profile and biological activity is highly consistent.

Other saponin fractions have been described. Fractions B3, B4 and B4b are described in EP 0436620. Fractions QA1-QA22 are described EP03632279 B2, Q-VAC (Nor-Feed, AS Denmark), Quillaja saponaria Molina Spikoside (Isconova AB, Ultunaallén 2B, 756 51 Uppsala, Sweden). Fractions QA-1, QA-2, QA-3, QA-4, QA-5, QA-6, QA-7, QA-8, QA-9, QA-10, QA-11, QA-12, QA-13, QA-14, QA-15, QA-16, QA-17, QA-18, QA-19, QA-20, QA-21, and QA-22 of EP 0 3632 279 B2, especially QA-7, QA-17, QA-18, and QA-21 may be used. They are obtained as described in EP 0 3632 279 B2, especially at page 6 and in Example 1 on page 8 and 9.

The saponin fractions described herein and used for forming adjuvants are often substantially pure fractions; that is, the fractions are substantially free of the presence of contamination from other materials. In particular aspects, a substantially pure saponin fraction may contain up to 40% by weight, up to 30% by weight, up to 25% by weight, up to 20% by weight, up to 15% by weight, up to 10% by weight, up to 7% by weight, up to 5% by weight, up to 2% by weight, up to 1% by weight, up to 0.5% by weight, or up to 0.1% by weight of other compounds such as other saponins or other adjuvant materials.

### ISCOM Structures

Saponin fractions may be administered in the form of a cage-like particle referred to as an ISCOM (Immune Stimulating COMplex). ISCOMs may be prepared as described in EP0109942B1, EP0242380B1 and EP0180546 B1. In particular embodiments a transport and/or a passenger antigen may be used, as described in EP 9600647-3 (PCT/SE97/00289).

### Matrix Adjuvants

In embodiments, the ISCOM is an ISCOM matrix complex. An ISCOM matrix complex comprises at least one saponin fraction and a lipid. The lipid is at least a sterol, such as cholesterol. In particular aspects, the ISCOM matrix complex also contains a phospholipid. The ISCOM matrix complexes may also contain one or more other immunomodulatory (adjuvant-active) substances, not necessarily a glycoside, and may be produced as described in EP0436620B1, which is incorporated by reference in its entirety herein.

In other aspects, the ISCOM is an ISCOM complex. An ISCOM complex contains at least one saponin, at least one lipid, and at least one kind of antigen or epitope. The ISCOM complex contains antigen associated by detergent treatment such that that a portion of the antigen integrates into the particle. In contrast, ISCOM matrix is formulated as an admixture with antigen and the association between ISCOM matrix particles and antigen is mediated by electrostatic and/or hydrophobic interactions.

According to one embodiment, the saponin fraction integrated into an ISCOM matrix complex or an ISCOM complex, or at least one additional adjuvant, which also is integrated into the ISCOM or ISCOM matrix complex or mixed therewith, is selected from fraction A, fraction B, or fraction C of Quillaja saponaria, a semipurified preparation of Quillaja saponaria, a purified preparation of Quillaja saponaria, or any purified sub-fraction e.g., QA 1-21.

In particular aspects, each ISCOM particle may contain at least two saponin fractions. Any combinations of weight % of different saponin fractions may be used. Any combination of weight % of any two fractions may be used. For example, the particle may contain any weight % of fraction A and any weight % of another saponin fraction, such as a crude saponin fraction or fraction C, respectively. Accordingly, in particular aspects, each ISCOM matrix particle or each ISCOM complex particle may contain from 0.1 to 99.9 by weight, 5 to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60% by weight, or 50% by weight of one saponin fraction, e.g. fraction A and the rest up to 100% in each case of another saponin e.g. any crude fraction or any other faction e.g. fraction C. The weight is calculated as the total weight of the saponin fractions. Examples of ISCOM matrix complex and ISCOM complex adjuvants are disclosed in U.S Published Application No. 2013/0129770, which is incorporated by reference in its entirety herein.

In particular embodiments, the ISCOM matrix or ISCOM complex comprises from 5-99% by weight of one fraction, e.g. fraction A and the rest up to 100% of weight of another fraction e.g. a crude saponin fraction or fraction C. The weight is calculated as the total weight of the saponin fractions.

In another embodiment, the ISCOM matrix or ISCOM complex comprises from 40% to 99% by weight of one fraction, e.g. fraction A and from 1% to 60% by weight of another fraction, e.g. a crude saponin fraction or fraction C. The weight is calculated as the total weight of the saponin fractions.

In yet another embodiment, the ISCOM matrix or ISCOM complex comprises from 70% to 95% by weight of one fraction e.g., fraction A, and from 30% to 5% by weight of another fraction, e.g., a crude saponin fraction, or fraction C. The weight is calculated as the total weight of the saponin fractions. In other embodiments, the saponin fraction from Quillaja saponaria Molina is selected from any one of QA 1-21.

In addition to particles containing mixtures of saponin fractions, ISCOM matrix particles and ISCOM complex particles may each be formed using only one saponin fraction. Compositions disclosed herein may contain multiple particles wherein each particle contains only one saponin fraction. That is, certain compositions may contain one or more different types of ISCOM-matrix complexes particles and/or one or more different types of ISCOM complexes particles, where each individual particle contains one saponin fraction from Quillaja saponaria Molina, wherein the saponin fraction in one complex is different from the saponin fraction in the other complex particles.

In particular aspects, one type of saponin fraction or a crude saponin fraction may be integrated into one ISCOM matrix complex or particle and another type of substantially pure saponin fraction, or a crude saponin fraction, may be integrated into another ISCOM matrix complex or particle. A composition or vaccine may comprise at least two types of complexes or particles each type having one type of saponins integrated into physically different particles.

In the compositions, mixtures of ISCOM matrix complex particles and/or ISCOM complex particles may be used in which one saponin fraction Quillaja saponaria Molina and another saponin fraction Quillaja saponaria Molina are separately incorporated into different ISCOM matrix complex particles and/or ISCOM complex particles.

The ISCOM matrix or ISCOM complex particles, which each have one saponin fraction, may be present in composition at any combination of weight %. In particular aspects, a composition may contain 0.1% to 99.9% by weight, 5% to 95% by weight, 10% to 90% by weight, 15% to 85% by weight, 20% to 80% by weight, 25% to 75% by weight, 30% to 70% by weight, 35% to 65% by weight, 40% to 60% by weight, 45% to 55% by weight, 40 to 60% by weight, or 50% by weight, of an ISCOM matrix or complex containing a first saponin fraction with the remaining portion made up by an ISCOM matrix or complex containing a different saponin fraction. In aspects, the remaining portion is one or more ISCOM matrix or complexes where each matrix or complex particle contains only one saponin fraction. In other aspects, the ISCOM matrix or complex particles may contain more than one saponin fraction.

In particular compositions, the only saponin fraction in a first ISCOM matrix or ISCOM complex particle is Fraction A and the only saponin fraction in a second ISCOM matrix or ISCOM complex particle is Fraction C.

In embodiments, the Fraction A of Quillaja Saponaria Molina accounts for at least about 80%, 81%, 82%, 83%, 84%, 85%, 86%, 87%, 88%, 89%, 90%, 91%, 92%, 93%, 94%, 95%, 96%, 97%, 98%, or 99% by weight, and fraction C of Quillaja Saponaria Molina accounts for the remainder, respectively, of the sum of the weights of fraction A of Quillaja Saponaria Molina and fraction C of Quillaja Saponaria Molina in the adjuvant.

Preferred compositions comprise a first ISCOM matrix containing Fraction A and a second ISCOM matrix containing Fraction C, wherein the Fraction A ISCOM matrix constitutes about 70% per weight of the total saponin adjuvant, and the Fraction C ISCOM matrix constitutes about 30% per weight of the total saponin adjuvant. In another preferred composition, the Fraction A ISCOM matrix constitutes about 85% per weight of the total saponin adjuvant, and the Fraction C ISCOM matrix constitutes about 15% per weight of the total saponin adjuvant. In another preferred composition, the Fraction A ISCOM matrix constitutes about 92% per weight of the total saponin adjuvant, and the Fraction C ISCOM matrix constitutes about 8% per weight of the total saponin adjuvant. Thus, in certain compositions, the Fraction A ISCOM matrix is present in a range of about 70% to about 85%, and Fraction C ISCOM matrix is present in a range of about 15% to about 30%, of the total weight amount of saponin adjuvant in the composition. In certain compositions, the Fraction A ISCOM matrix is present in a range of about 70% to about 92%, and Fraction C ISCOM matrix is present in a range of about 8% to about 30%, of the total weight amount of saponin adjuvant in the composition. In embodiments, the Fraction A ISCOM matrix accounts for 50-96% by weight and Fraction C ISCOM matrix accounts for the remainder, respectively, of the sums of the weights of Fraction A ISCOM matrix and Fraction C ISCOM in the adjuvant. In a particularly preferred composition, referred to herein as MATRIX-MIM, the Fraction A ISCOM matrix is present at about 85% and Fraction C ISCOM matrix is present at about 15% of the total weight amount of saponin adjuvant in the composition. MATRIX-MIM may be referred to interchangeably as Matrix-M1.

Exemplary QS-7 and QS-21 fractions, their production and their use is described in U.S Pat. Nos. 5,057,540; 6,231,859; 6,352,697; 6,524,584; 6,846,489; 7,776,343, and 8,173,141, which are incorporated by reference herein.

In embodiments, other adjuvants may be used in addition or as an alternative. The inclusion of any adjuvant described in Vogel et al., “A Compendium of Vaccine Adjuvants and Excipients (2nd Edition),” herein incorporated by reference in its entirety for all purposes, is envisioned within the scope of this disclosure. Other adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, MDP compounds, such as thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/TWEEN® polysorbate 80 emulsion. In embodiments, the adjuvant may be a paucilamellar lipid vesicle; for example, NOVASOMES®. NOVASOMES® are paucilamellar nonphospholipid vesicles ranging from about 100 nm to about 500 nm. They comprise BRIJ® alcohol ethoxylate 72, cholesterol, oleic acid and squalene. NOVASOMES® have been shown to be an effective adjuvant (see, U.S. Pat. Nos. 5,629,021, 6,387,373, and 4,911,928.

### EXAMPLES

### Example 1: Pseudovirus Based Neutralization Assay for SARS-CoV-2 Variants: A Rapid Cost-Effective BSL2 Based High Throughput Assay Useful for Vaccine Immunogenicity Evaluation

Introduction: We have discovered a novel pseudovirus based neutralization assay for evaluating the immunogenicity of vaccines against SARS-CoV-2 variants. The assay is a BSL-2 based assay, which is cost effective, leads to a rapid turnaround, and enables evaluation of immunogenicity of SARS-CoV-2 vaccines (e.g., neutralizing antibody production) against emerging SARS-CoV-2 variants. This assay is a surrogate for live virus neutralization assay and does not require use of a BSL-3 laboratory.

Methods: We have developed a BSL-2 based pseudovirus based neutralization assay against SARS-CoV-2 prototype and variants (Omicron BA.1 and BA.2). This assay is suitable for measurement of neutralization ability in clinical samples (serum from human subjects infected with SARS-CoV-2 or immunized with COVID-19 vaccines). Significantly, this assay may be performed in a BSL-2 laboratory rather than a BSL-3 laboratory. This assay may be used to evaluate the ability of SARS-CoV-2 vaccines to stimulate an immune response against prototype SARS-CoV-2 viruses. Prototype SARS-CoV-2 viruses comprise the SARS-CoV-2 S protein having the amino acid sequence of SEQ ID NO: 2. This assay can also be utilized to evaluate the ability of SARS-CoV-2 vaccines to stimulate an immune response against prototype SARS-CoV-2 viruses, including the Omicron BA.1, BA.2, and BA.5 variants. This assay can further be utilized to evaluate the ability of human sera to neutralize SARS-CoV-2 and SARS-CoV-2 variants.

SARS-CoV-2 pseudoviruses expressing SARS-CoV-2 spike proteins were produced by co-transfecting cells with plasmids encoding lentiviral backbone expressing luciferase reporter, plasmids encoding non-surface proteins for lentiviral production and plasmid expressing SARS-CoV-2 spike protein (either Prototype strain or Omicron strain) under the control of a mammalian promoter.

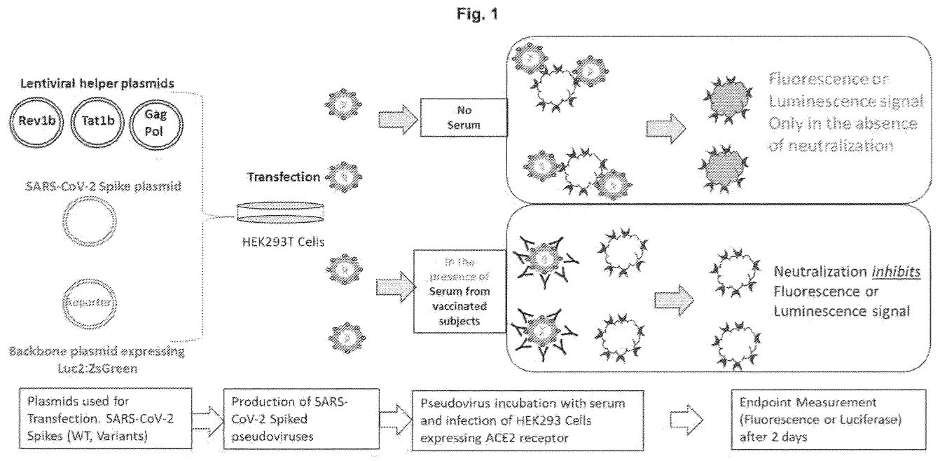

A schematic of the pseudovirus neutralization assay utilized in this example is found in FIG. 1.

SARS-CoV-2 pseudoviruses were produced by transfecting HEK293T cells with (i) a plasmid encoding a lentiviral backbone expressing a reporter protein (e.g., luciferase or ZsGreen); (ii) lentiviral helper plasmids (e.g., plasmids expressing Rev1b, Tat1b, Gag, and Pol); and (iii) a plasmid expressing a SARS-CoV-2 S glycoprotein. The plasmids expressed the gene of interest under the control of a cytomegalovirus (CMV) promoter. Pseudoviruses were harvested from the cell culture supernatant.

Pseudoviruses were then incubated from serum of patients who had recovered from a SARS-CoV-2 infection or patients that were immunized with the SARS-CoV-2 vaccine, NVX-CoV2373. As a control, pseudoviruses were incubated with cell culture medium without serum (virus-only “VC” controls).

The pseudoviruses were used to infect cells expressing the ACE2 receptor and incubated at 37° C. for 48 to 72 hours. As a negative control, cells were infected with cell culture medium in the absence of Pseudovirus. The luminescence of the cells (from luciferase) was measured. A reduction of luciferase signal indicated a neutralization of infection.

The dose dependence of luminescence on pseudovirus level was determined by infecting cells expressing ACE2 receptor with different amounts of pseudovirus, and detecting luciferase luminescence (RLU) 2 days post-infection.

An optimal cell line for pseudovirus-based neutralization assays was determined. The following cells were evaluated: Vero-E6 (African Green Monkey kidney cells), A549/ACE2-TMPRESS (human lung epithelial cells expressing ACE2 and TMPRESS), and 293T/ACE2 cells (human kidney epithelial cells expressing ACE2). Cells were infected with the pseudoviruses and luminescence two days after infection was measured. The infectivity of the cell lines was determined by TCID50 quantitation. The suitability of the cells lines for the pseudovirus neutralization assay was further confirmed by infecting 293T/ACE2 and A549/ACE2-TMPRESS cells with Omicron BA.1/BA.5 pseudoviruses in the presence or absence of serum, followed by measurement of luminescence. Controls were cells infected but not treated with serum (virus control), and cells without pseudovirus (cell control). Percent (%) inhibition/neutralization of pseudovirus infection by the serum relative to virus control was determined.

Pseudovirus-based neutralization titer using different amounts of pseudovirus was assessed to optimize the assay procedure.

The kinetics of the pseudovirus neutralization assay endpoint was evaluated by measuring luminescence at 48 and 72 hours, and comparing the signal to background (S/B) ratios.

Assay Quality—Precision: Quality control (QC) samples were tested by 2 operators (analysts) on 2 different days, and duplicates of each sample were tested in the same assay. For prototype/Wuhan strain, high-, medium-, and low-quality QC samples were used. For variants, 2 or 3 QC samples were used.

Assay Quality—Linearity: One sample was serially diluted (6 times, 4-fold dilution series), and evaluated in duplicate by 2 different operators.

Correlation Analyses with live virus-based microneutralization (MN) assay: Samples were tested in a validated live virus MN assay (360biolabs) (n=13), followed by comparison of data with from the pseudovirus neutralization assay using linear regression analysis.

Correlation with anti-S IgG antibodies and hACE2 binding inhibition—Samples were tested in a validated anti-S IgG assay (n=15) or validated hACE2 binding inhibition assay (n=8), followed by comparison of data with pseudovirus neutralization assay results using linear regression analysis.

Clinical Utility: To assess clinical utility of the assay, clinical serum samples from participants administered the NVX-CoV2373 vaccine were tested against prototype/Wuhan strain and variants (Omicron BA.1, BA.2, BA.5) using the pseudovirus neutralization assay. Neutralizing antibody response against prototype/Wuhan strain and variants in clinical serum samples was profiled. To demonstrate the effects of vaccine boosters on neutralizing antibody responses (using PNT), serum from a patient before and after the patient was administered a booster dose of the NVX-CoV2373, was evaluated in the pseudovirus neutralization assay.

Results: Pseudovirus based neutralization assay in 96-well plate-based format was developed and optimized in dose (TCID₅₀, amount of pseudovirus/well) and kinetics (assay duration) experiments in HEK293/ACE2 cells. Assay positive and negative controls demonstrated a wide dynamic range. Neutralization data from pseudovirus assays show robust correlation with validated Anti-rSpike IgG levels and ACE2 inhibition titers (prototype). Currently, the pseudovirus assay against Omicron BA.5 is being developed and sera from patients who received the NVX-CoV2373 vaccine are being evaluated in the assay.

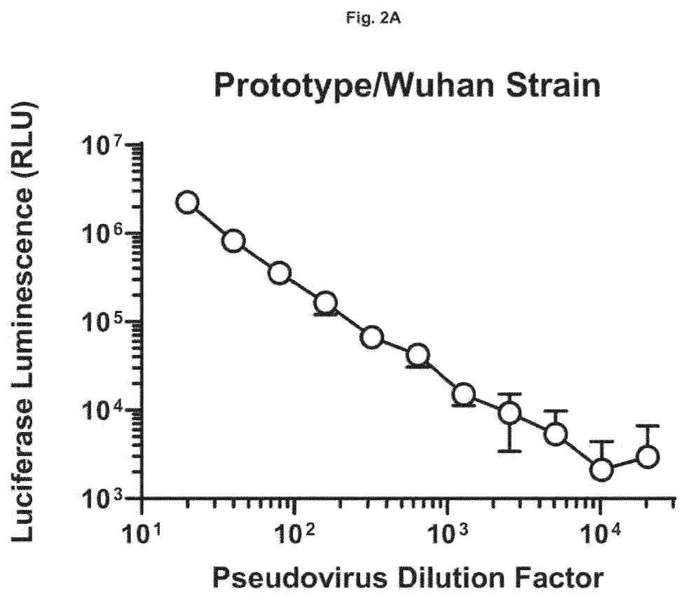

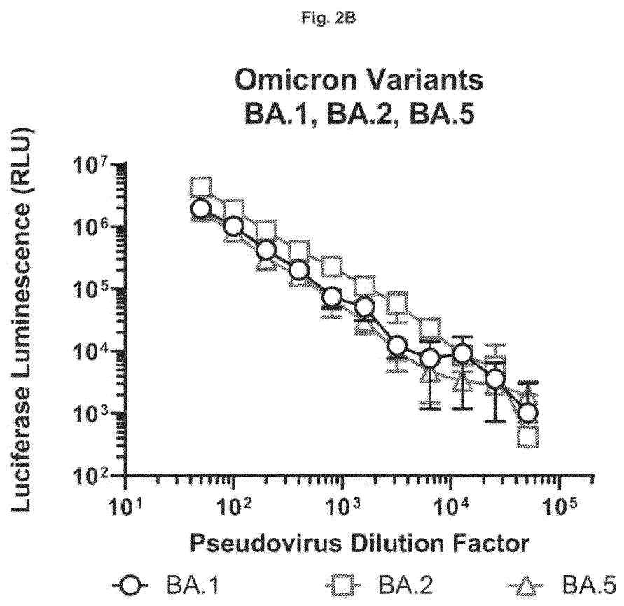

The luminescence signal from the Pseudovirus neutralization assay exhibited a dose dependence on pseudovirus amount used for infection (FIG. 2A, FIG. 2B). The luciferase endpoint showed a robust dynamic range and user-friendliness (ease of use and throughput).

Results—Effect of ACE2 Expressing Cell Line on Pseudovirus Infection: To evaluate the suitability of cell lines for pseudovirus infection, three different cell lines (Vero E6-African Green Monkey Kidney cell line, A549/ACE2-TMPRESS—Human lung epithelial cell line expressing ACE2 and TMPRESS, 293T-ACE2 Human Kidney epithelial cell line expressing ACE2) were infected with various amounts of pseudoviruses expressing a SARS-CoV-2 S protein from the prototype strain (also referred to as “prototype/Wuhan”) or an S protein from a SARS-CoV-2 variant (Omicron BA.1, BA.2, BA.5). Luciferase levels in the infected cells after 2 days are shown on the Y-axis. TCID50/mL of the pseudoviruses calculated by the luciferase levels are shown in the table below.

| | TCID50/mL | | | |
|---|---|---|---|---|
| Cell Line | Wuhan | BA.1 | BA.2 | BA.5 |
| Vero-E6 | 20,380 | 17,820 | 46,240 | 53,120 |
| A549/ACE2- | 498,600 | 398,480 | 367,420 | 411,280 |
| TMPRESS | | | | |
| 293T/ACE2 | 543,700 | 663,660 | 1,249,340 | 834,400 |

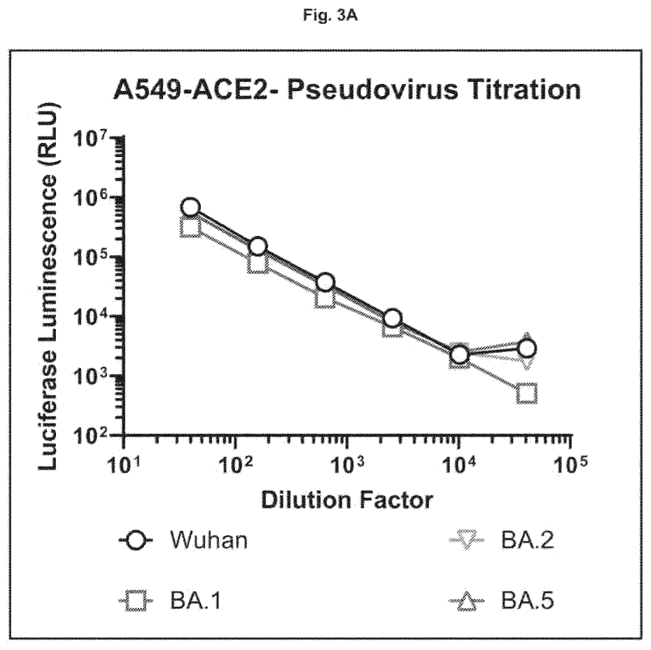

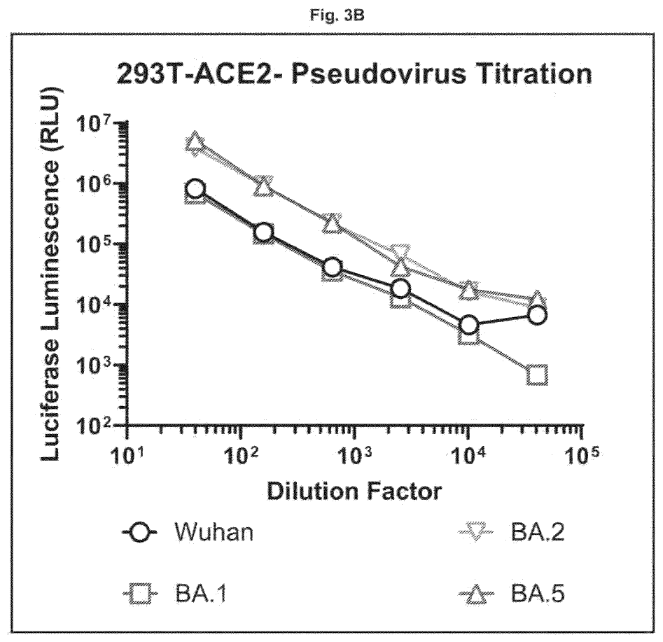

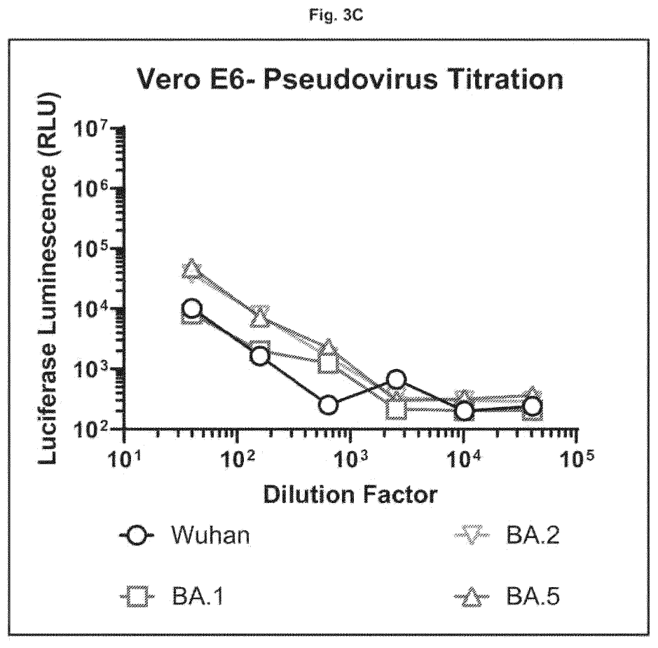

Cell lines expressing ACE2 (e.g., Vero-E6, A549-ACE2-TMPRESS, and 293T-ACE2 cell lines) had a differential susceptibility to infection with pseudoviruses. (FIGS. 3A-3C). Vero-E6 cells were less suitable for pseudovirus infection than the other cell lines evaluated, regardless of the identity of the expressed SARS-CoV-2 S protein. (FIG. 3C). The ability of pseudovirus expressing a SARS-CoV-2 S protein from the prototype or Wuhan strain to infect 293T-ACE2 cells and A549-ACE2-TMPRESS cells was similar. (FIG. 3A, FIG. 3B). However, the infectivity of the Pseudovirus was higher in 293T/ACE2 cells when the Pseudovirus expressed a SARS-CoV-2 S protein from a SARS-CoV-2 Omicron strain.

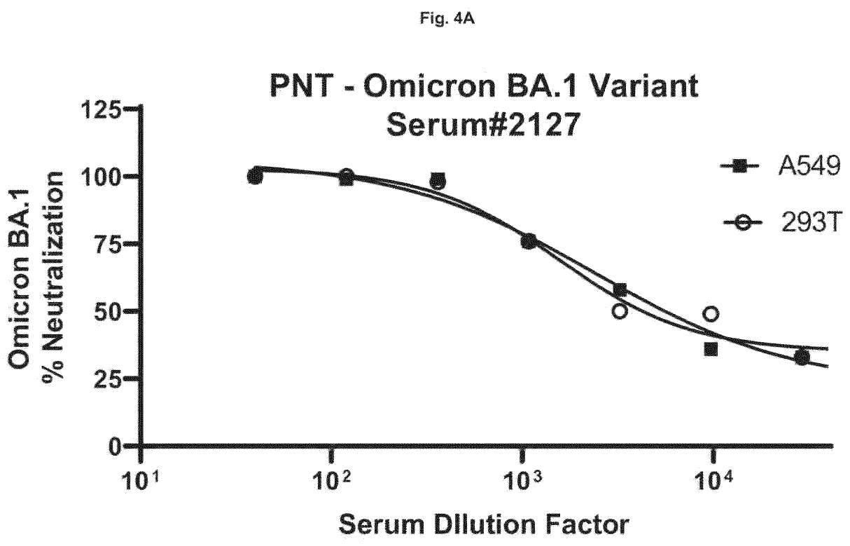

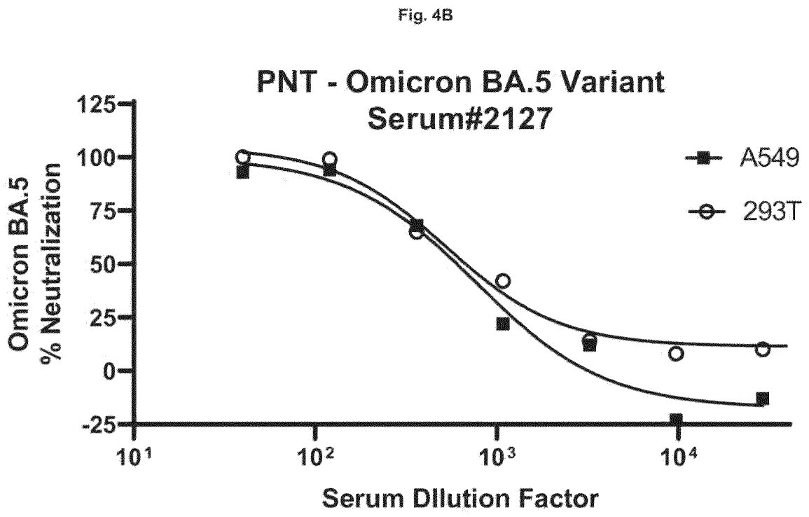

To compare the suitability of 293T/ACE2 and A549/ACE2 cell lines for Pseudovirus neutralization assay (PNT), cells were infected with Omicron BA.1 and BA.5 pseudoviruses in the presence/absence of increasing dilutions of serum (#2127), followed by luciferase measurement as described in the methods. Cells either infected with the pseudoviruses (BA.1 and BA.5) in the absence of test serum (#2127) (Virus control) and no pseudoviruses (Cell control) were used as controls. Percent (%) inhibition compared to the virus control are shown on the Y-axis. Both cell lines show similar (overlapping) luciferase inhibition curves (ID50). FIGS. 4A-4B show the ID50 of pseudovirus infection for 293T cells and A549/ACE2-TMPRESS cells. The table below shows the pseudovirus comparable neutralization titers (ID50) for different serum samples. This data shows that both of these cell lines is suitable for use in pseudovirus neutralization assays.

| | |
|---|---|
| PNT ID₅₀ | PNT ID₅₀ |
| BA.1 Titer | BA.5 Titer |

| Sample ID | 293T | A549 | 293T | A549 |
|---|---|---|---|---|
| Serum #8522 | <40 | <40 | NT | NT |
| Serum #2127 | 4423 | 2905 | 674 | 603 |
| Serum #2124 | 5782 | 5811 | 1950 | 1524 |
| Serum #7781 | <40 | <40 | 40 | 43 |
| Serum #8061 | NT | NT | 102 | 126 |

To evaluate the effect of dose and time on PNT, four (4) test serum samples were evaluated in PNT, with increasing amounts of pseudovirus (prototype/Wuhan strain): 50, 125 and 250 TCID50/well. ID50 values of the serum are plotted on the Y-axis. Decreasing neutralization ability as measured by ID50 levels with increasing amounts of pseudoviruses used for infection was observed. The table below shows the effect of pseudovirus dose (ranging from 50 to 3.13 uL/well) and kinetics (48 hours (48 h) vs 72 hours (72 h) assay endpoint) on signal/background (S/B) levels in 293T/ACE2 cells.

Signal/Background Ratio

| | | |
|---|---|---|
| Virus per | Wuhan | BA.1 |

| well (μL) | 48 h | 72 h | 48 h | 72 h |
|---|---|---|---|---|
| 50 | 735 | 329 | 636 | 427 |
| 25 | 339 | 161 | 224 | 147 |
| 12.5 | 152 | 39 | 65 | 72 |
| 6.25 | 97 | 32 | 32 | 34 |
| 3.13 | 66 | 15 | 18 | 18 |

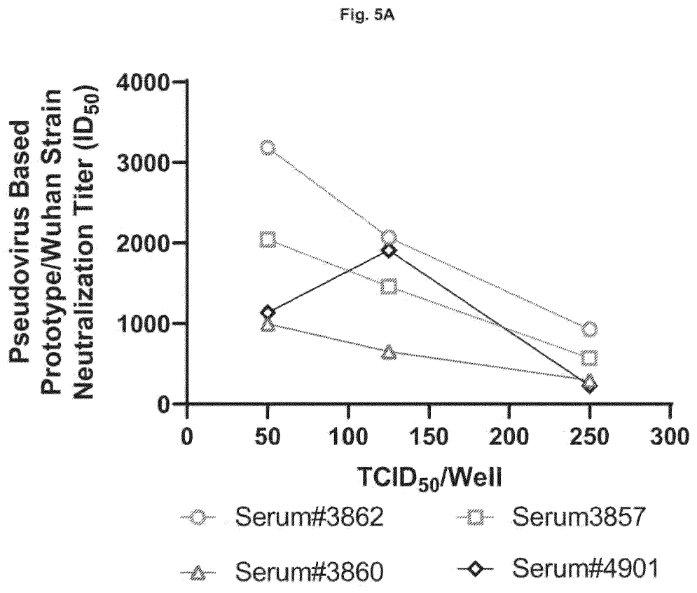

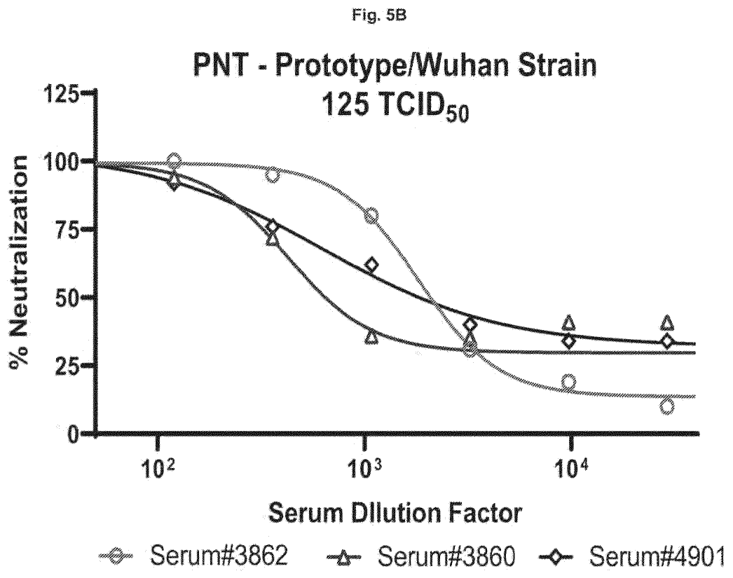

As the 48 h timepoint was found to provide better S/B ratio for all pseudovirus amounts and strains, it was selected for subsequent experiments. FIGS. 5A-5B show the luminescence output 48 hours (FIG. 5A) and 72 hours (FIG. 5B) after cells are infected with Pseudovirus. Three out of four serum samples showed dose dependence of pseudovirus-based neutralization titer (PNT ID50). (FIGS. 5A-5B).

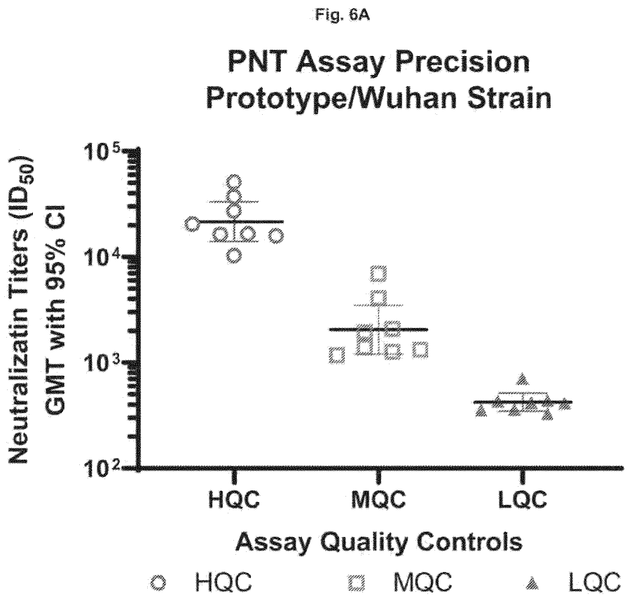

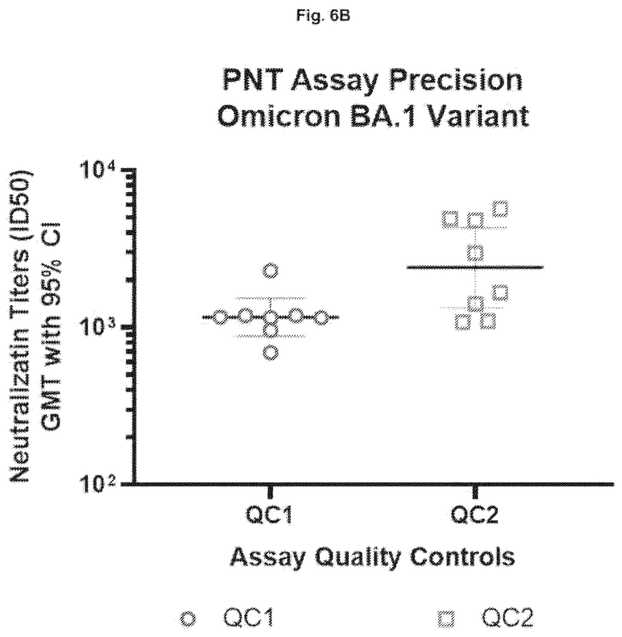

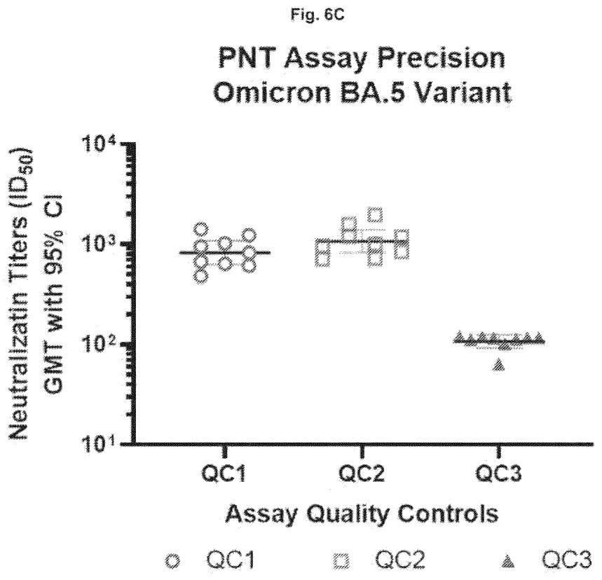

To evaluate precision of the assay, quality control serum samples were evaluated in the PNT by two different operators on two different days. Duplicate samples were also tested in the same assay to evaluate the intra-assay precision. Neutralization titers (ID50) are plotted on the Y-axis (GMT, 95% CI). FIGS. 6A-6C shows the inter- and intra-assay precision of the pseudovirus neutralization assay. FIG. 6A shows precision when the pseudovirus encodes a SARS-CoV-2 S protein from the prototype strain. FIG. 6B shows precision when the pseudovirus encodes a SARS-CoV-2 S protein from the Omicron BA.1 strain. FIG. 6C shows precision when the pseudovirus encodes a SARS-CoV-2 S protein from the Omicron BA.5 strain.

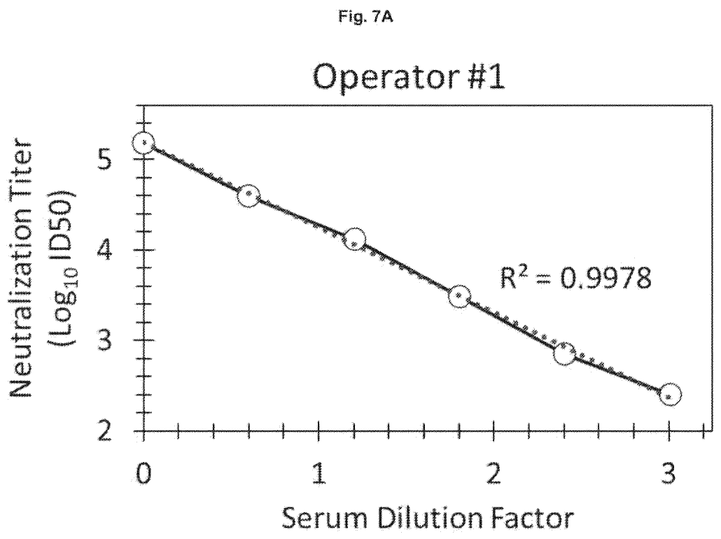

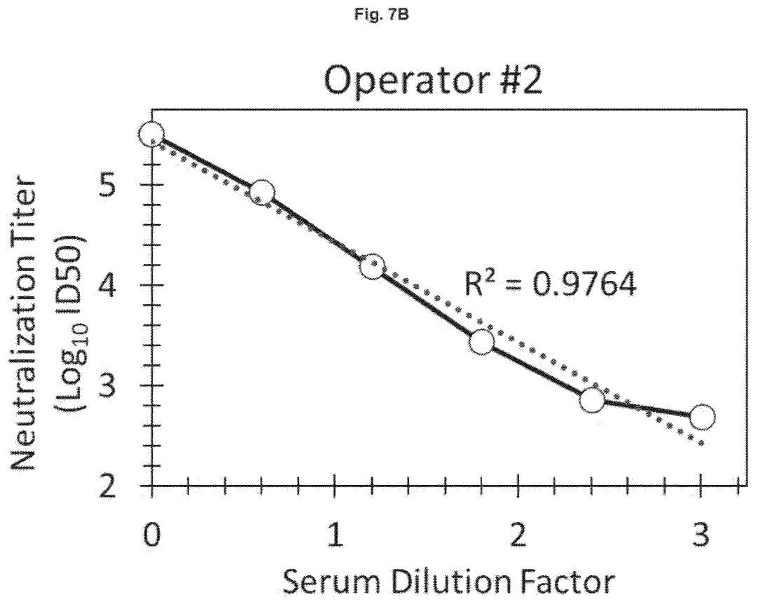

To evaluate the linearity of PNT, a test serum (Serum#2127) was serially diluted (6-times, 4-fold dilution series), followed by evaluation in PNT (prototype/Wuhan strain) in duplicates at each dilution, by two different operators (1 and 2) as described in the methods. Neutralization titers (ID50) of the different dilutions are plotted on Y-axis. FIGS. 7A-7B show dose proportional neutralization with serum dilutions by different operators (FIG. 7A—Operator#1, FIG. 7B—Operator#2). Assay linearity was demonstrated for prototype/Wuhan strain-based pseudovirus (R²=0.9978 and 0.9764 for the two operators).

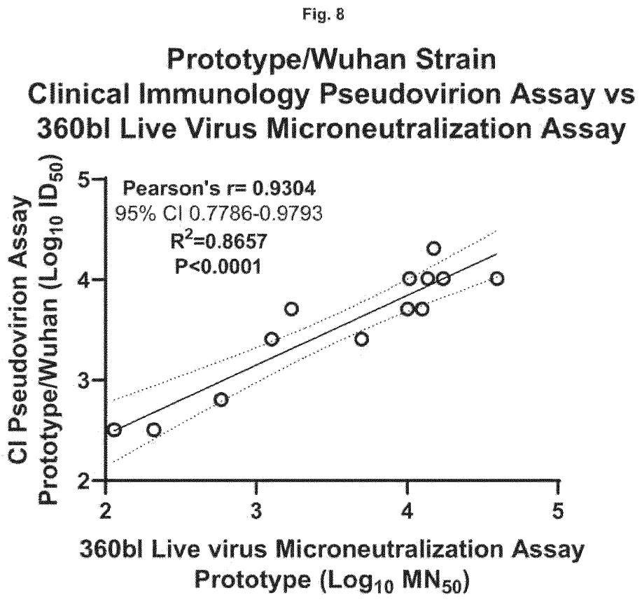

FIG. 8 shows that results from the pseudovirus neutralization assay are significantly correlated for prototype/Wuhan strain with the live virus MN assay (Pearson's r=0.9304, R²=0.8657, P<0.0001). Test serum samples (n=13) from a clinical study were evaluated in PNT against prototype/Wuhan strain as per the method mentioned above, followed by regression analysis with a validated live virus microneutralization assay. Analysis was performed using GraphPad Prism software (9.3.1). Dotted line shows 95% confidence interval (CI). Pearson correlation coefficient (Pearson r) and two tailed P values are shown in the figures.

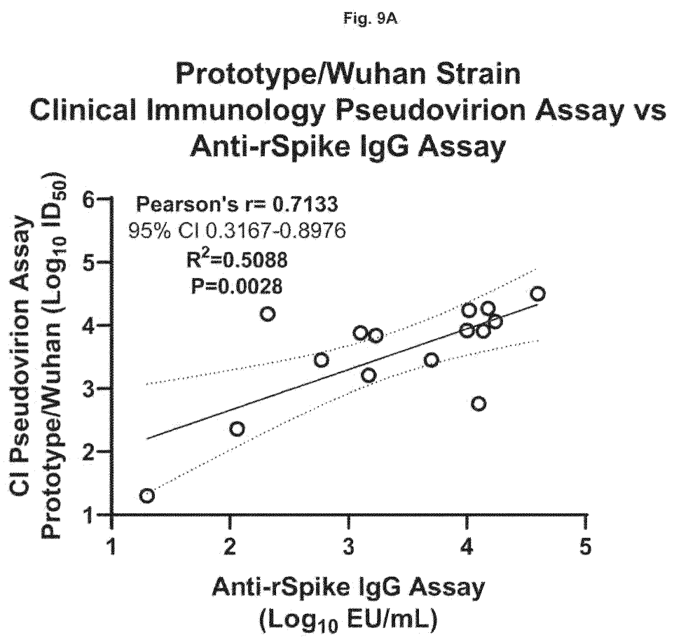

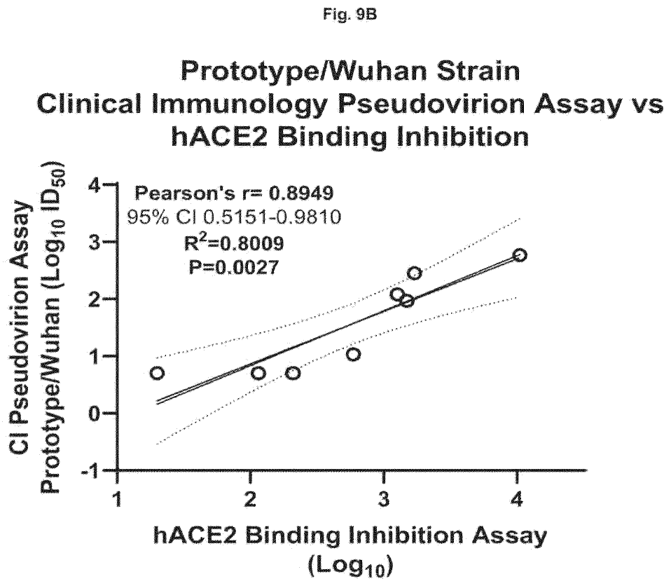

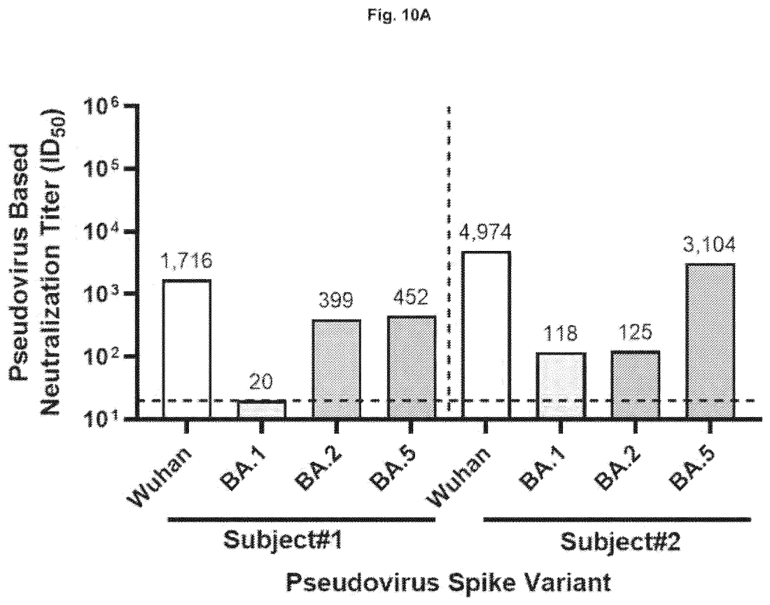

FIGS. 9A-9B show that the ability of a serum sample to resist pseudovirus infection is correlated with the amount of anti-SARS-CoV-2 S protein immunoglobulin (Pearson's r=0.7133, R²=0.5088, P=0.0028) (FIG. 9A) and hACE2 binding inhibition titers (Pearson's r=0.8949, R²=0.8009, P=0.0027) (FIG. 9B). Analysis was performed using GraphPad Prism software (9.3.1). Dotted line shows 95% confidence interval (CI). Pearson correlation coefficient (Pearson r) and two tailed P values are shown in the figures

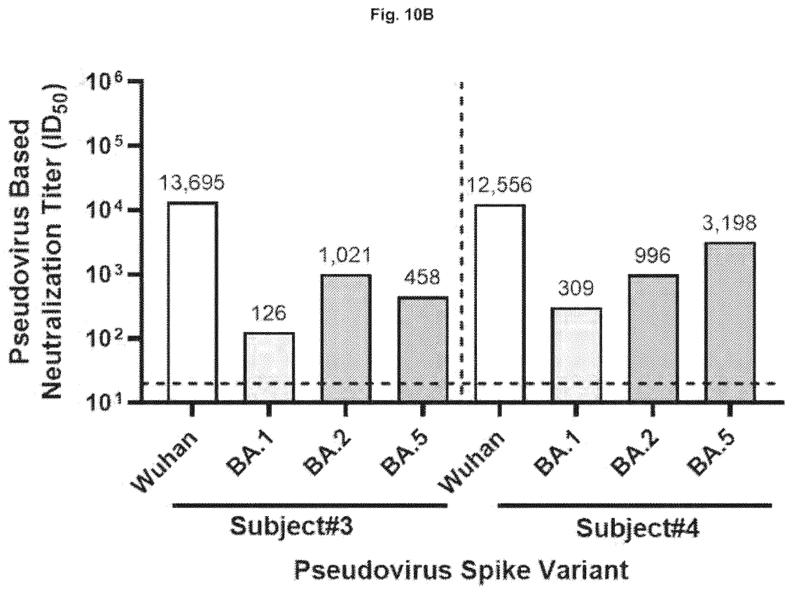

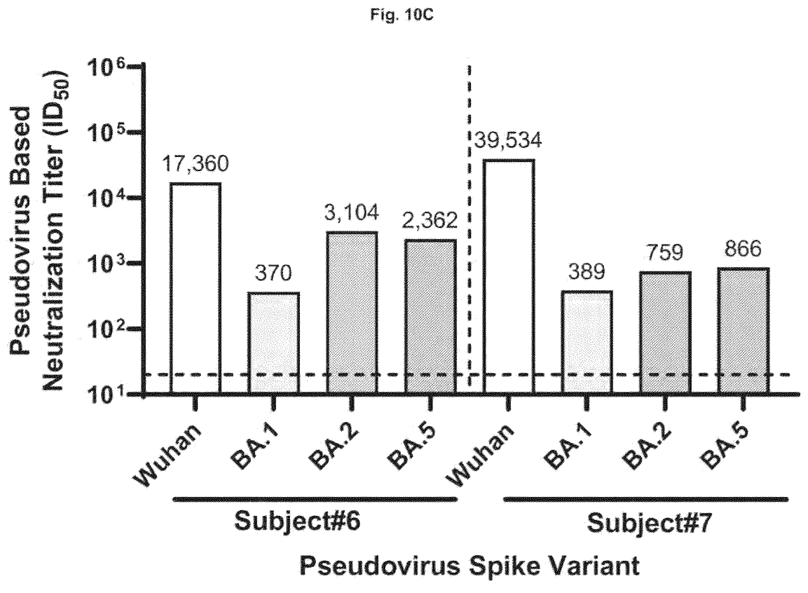

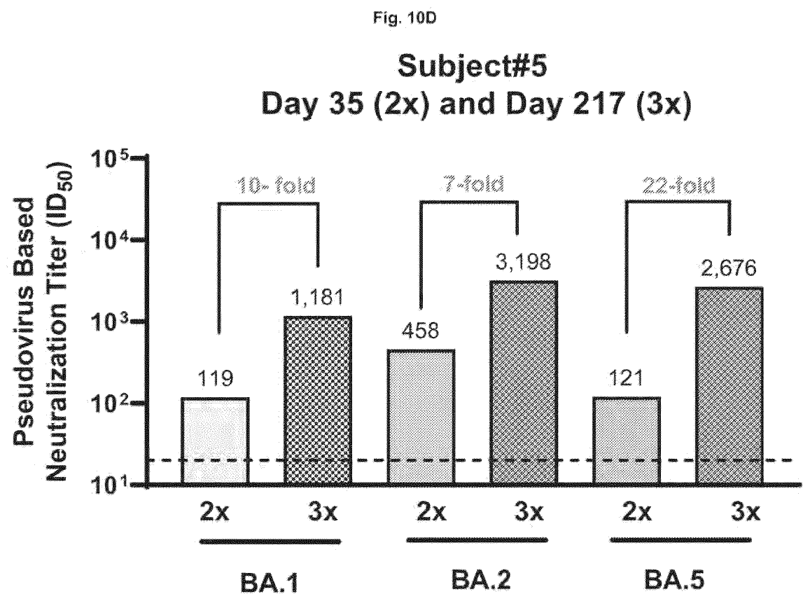

FIGS. 10A-C show the neutralization titers (ID50) for serum from patients 1 and 2 (FIG. 10A, patients 3 and 4 (FIG. 10B), and patients 6 and 7 (FIG. 10C). FIG. 10D shows the neutralization titers of patient #5 14 days after administration of a primary NVX-CoV2373 vaccine series (“2x” in FIG. 10D) and after administration of a NVX-CoV2373 booster (“3x” in FIG. 10D). The primary NVX-CoV2373 vaccine series was administered on days 0 and 21. The serum was collected 28 days after booster dose shown as 3x on the X-axis [prime series (day 0 and 21)+booster (day 189)].

Abbreviations: GMT, geometric mean titer; hACE2, human angiotensin converting enzyme 2; HQC, high quality control sample; IgG, immunoglobulin G; LQC, low quality control sample; MN, microneutralization; MQC, mid quality control sample; N/A, not applicable; NC, negative control; PNT, pseudovirus neutralization assay; RLU, relative luminescence units; rS, recombinant spike protein; R2, coefficient of determination; SARS-CoV-2, severe acute respiratory syndrome coronavirus 2; SD, standard deviation; S-protein, spike protein.

Conclusions: The pseudovirus based neutralization assay described herein for determining the immunogenicity of COVID-19 vaccines provides a cost-effective high throughput alternative to BSL3 based microneutralization assays with rapid turnaround and will further enable discovery and development of effective vaccines against emerging COVID-19 variants.

### Example 2: Multiplexed Detection of Neutralizing Antibody Responses Against Different SARS-CoV-2 Strains in the Same Assay Plate Using Multiplexed Pseudovirus Assays

Introduction: With the continuous evolution of SARS-CoV-2 around the globe, different variants are being observed in circulation. With the limited amount of serum collected from human subjects either in the vaccine clinical trials, convalescent subjects or in the SARS-CoV-2 infected subjects, it is difficult to measure the neutralizing antibody responses against multiple variants simultaneously. Separate measurements of neutralizing antibodies against each variants increases cost, analysis turn around time and adds to the complexities to the logistics of sample analysis and interpretation to be clinically meaningful.

Methods: To address this issue, a multiplex pseudovirus neutralization assay was developed, using pseudoviruses expressing spike proteins for different strains of SARS-CoV-2 (for e.g., prototype Wuhan, Omicron BA.1 and BA5 strains) and different reporter proteins (RFP and GFP), followed by pseudovirus neutralization assay optimization and evaluation of serum samples positive for neutralization antibodies against SARS-CoV-2.

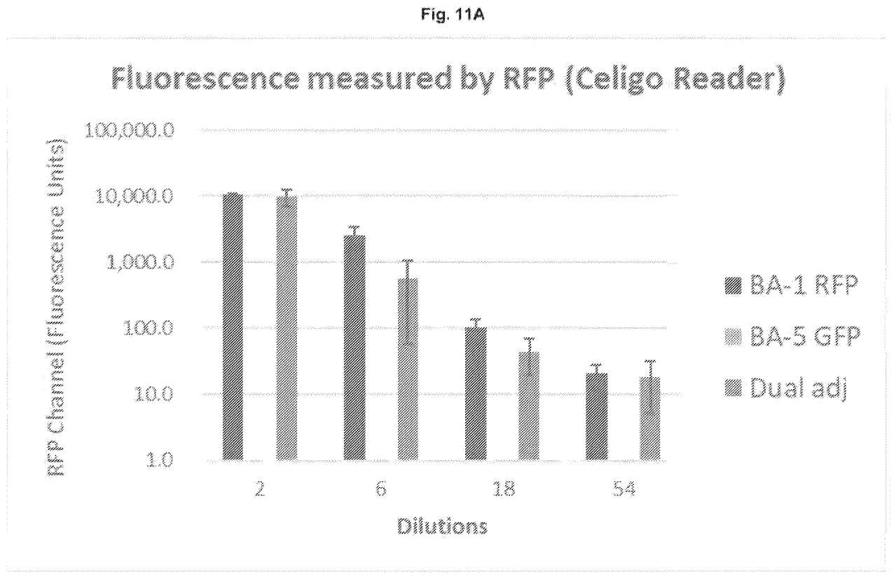

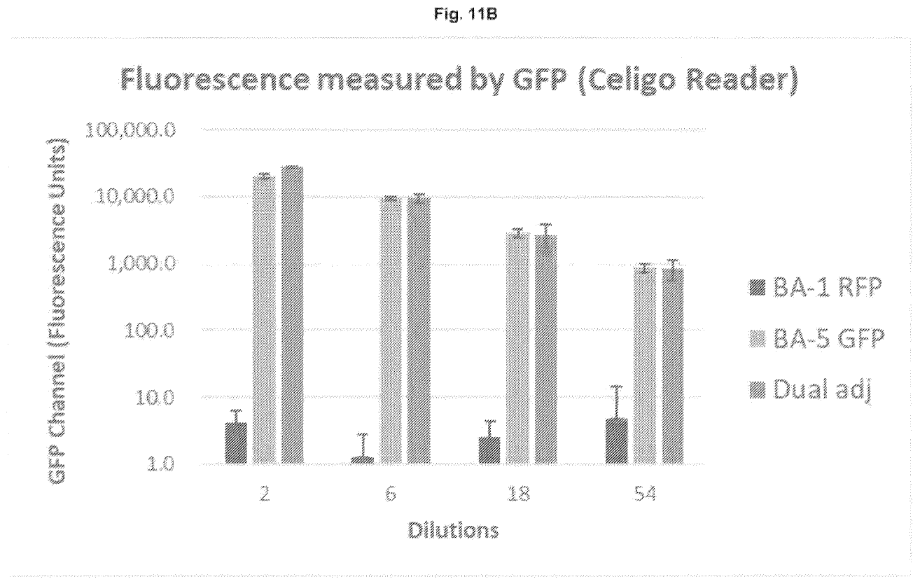

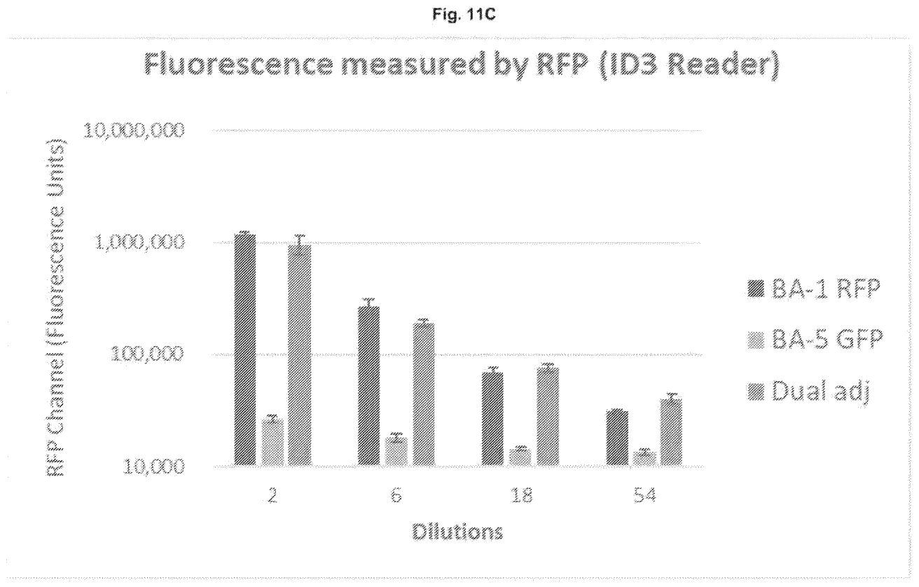

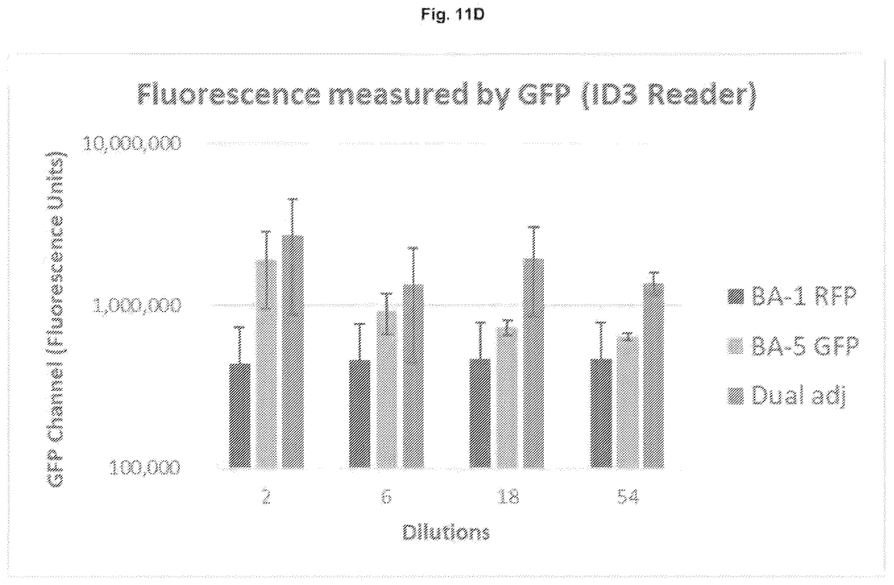

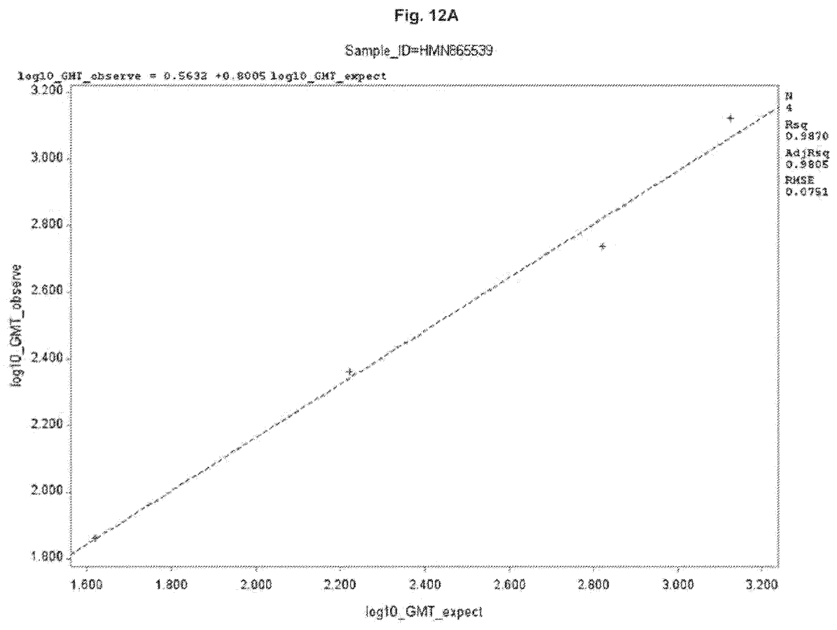

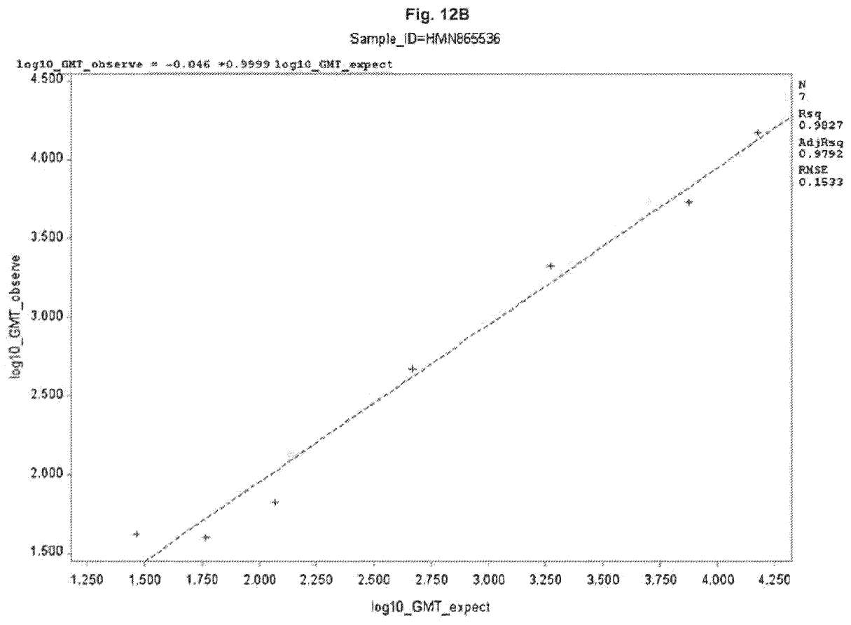

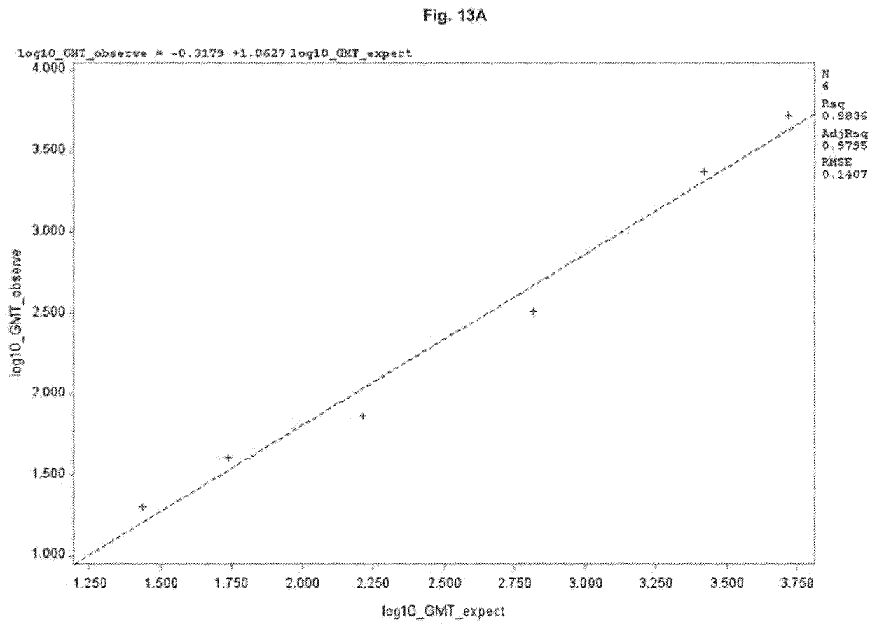

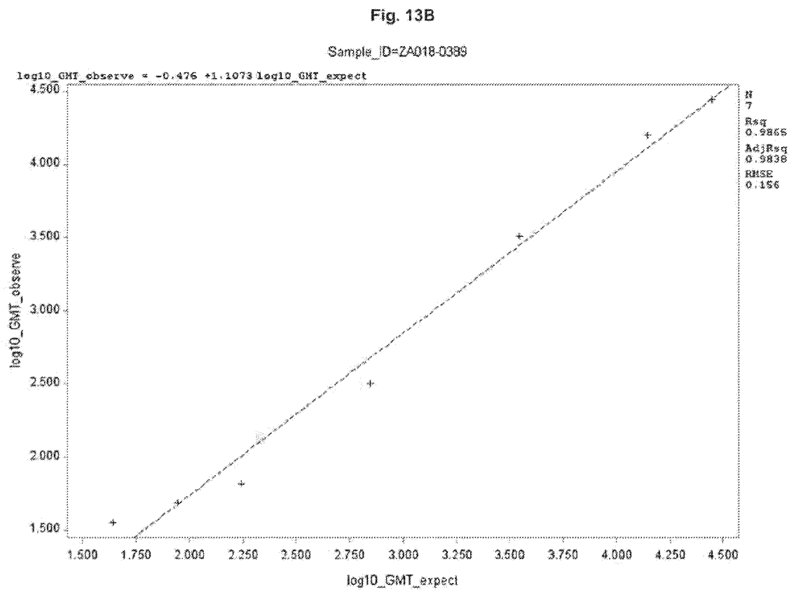

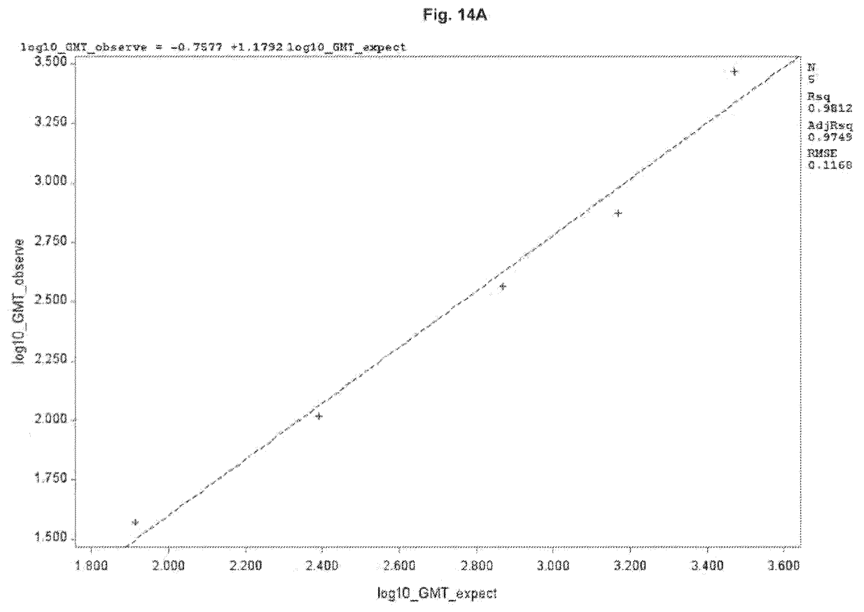

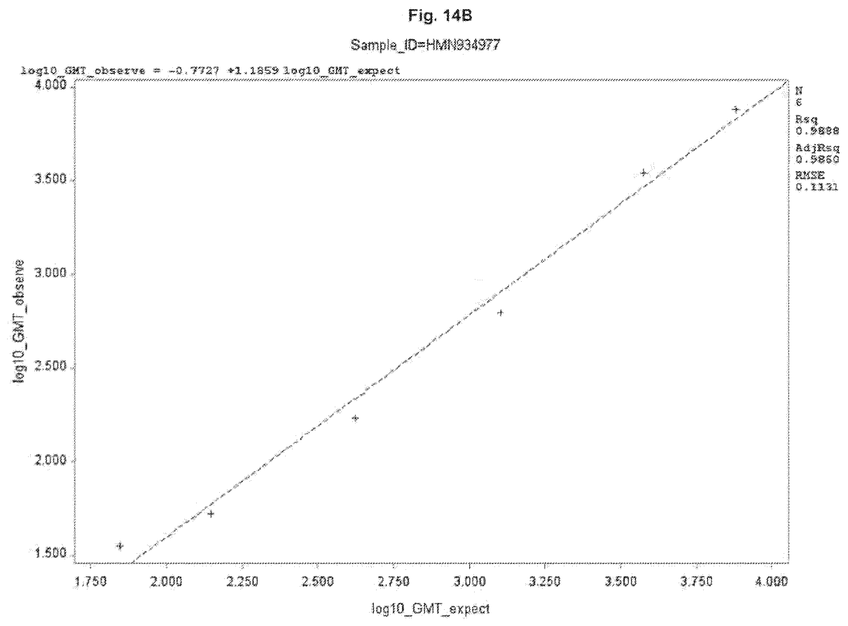

Results: In the first set of experiments, pseudoviruses expressing Omicron BA.1 spike protein along with RFP reporter protein (BA.1-RFP) and Omicron BA.5 spike protein along with GFP reporter protein (BA.5-GFP) were prepared, followed by infection of HEK293T/ACE2 cells (1.25×104 cells/well) in 96 well plates, either alone or in combination together at serial dilutions (ranging from 2-fold dilutions to 54-fold dilutions, using 3-fold dilution series). Cells were incubated at 37 C and 5% CO2 for 72 hours followed by measurement of fluorescence for GFP and RFP, using two different plate readers (Celigo and ID3 plate readers). Our observation suggested that, both the reporter protein (RFP and GFP) fluorescence can be observed in the same wells of 96 well plates with no/little interference (FIGS. 11A-11D).

To further test the feasibility of a multiplexing approach for measuring the neutralizing antibody responses in serum samples, convalescent serum samples from a commercial source, were heat inactivated (56° C. for 30 minutes), incubated with pseudoviruses expressing SARS-CoV-2 S glycoproteins from different strains and different reporters (BA.1 RFP and Wuhan-GFP) either alone or in combination together for 2 hours at 37 C. The serum-pseudovirus mixture was used to infect 293T/ACE2 cells in 96 wells plates in duplicates, followed by measurement of fluorescence for RFP and GFP after 72 hours. The table below shows the percent neutralization observed in individual pseudovirus neutralization tests (PNT) for either Omicron BA.1 RFP and BA.5 GFP and both of them together.

| | Percent (%) Neutralization* | | | | | | | |
|---|---|---|---|---|---|---|---|---|
| | Individual PNT assay | | | | Multiplex PNT assay | | | |
| Serum | BA1-RFP | | Wuhan-GFP | | BA1-RFP | | Wuhan-GFP | |
| samples** | Ave | SD | Ave | SD | Ave | SD | Ave | SD |
| HMN752127 | 65.7 | 0.6 | 73.1 | 1.6 | 57.4 | 1.2 | 74.2 | 4.4 |
| HMN752124 | 65.5 | 0.9 | 61.2 | 1.4 | 58.0 | 0.8 | 71.0 | 0.6 |
| 108522 | 61.2 | 0.2 | 53.2 | 0.2 | 56.9 | 1.9 | 67.5 | 0.4 |
| 107781 | 55.7 | 3.3 | 56.4 | 5.1 | 51.5 | 7.8 | 67.7 | 6.0 |

*Single dilution (1; 20) of the serum samples
**Commercially available serum samples from convalescent subjects

Data from this experiment showed the feasibility of measurement of neutralization activity present in the human serum samples in the multiplex pseudovirus assay in the same wells. Some of the advantages of this multiplex assay are 1. Low volume of test serum sample requirement. 2. Reduced assay cost (Reagent and labor cost) 3. Rapid turn around time 4. Improved efficiency of neutralization assay measurement.

### Example 3: Validation of Pseudovirus Based Neutralization Assay

Purpose: The pseudovirus based neutralization assay was validated to demonstrate its suitability for clinical sample tested.

Methods: Serum from patients immunized against SARS-CoV-2 was heat-inactivated at 56° C. for about 30 minutes. As a negative control, patient serum collected prior to the outbreak of the SARS-CoV-2 outbreak was utilized. Positive controls were human sera with high, medium, and low pseudovirus neutralization (PNT) titers. Each heat inactivated serum sample was diluted 1:10 in infection medium before use in the assay. Pseudovirus was added to the sample and incubated to allow virus-specific neutralizing antibodies to neutralize the virus. The pseudoviruses are replication-deficient Maloneymurine leukemia virus (MLV). The pseudoviruses expressed one of the following SARS-CoV-2 S glycoproteins: (i) a SARS-CoV-2 S glycoprotein, wherein amino acid 601 is glycine, as compared to the glycoprotein of SEQ ID NO: 2 (referred to as “Wuhan D614 pseudovirus”); (ii) a SARS-CoV-2 S glycoprotein, wherein amino acid 54 is V, amino acid 56 is deleted, amino acid 57 is deleted, amino acid 82 is I, amino acid 129 is D, amino acid 130 is deleted, amino acid 131 is deleted, amino acid 132 is deleted, amino acid 198 is deleted, amino acid 199 is I, the tripeptide EPE is inserted after amino acid 201, amino acid 534 is K, amino acid 601 is G, amino acid 642 is Y, amino acid 666 is K, amino acid 668 is H, amino acid 751 is K, amino acid 783 is Y, amino acid 843 is K, amino acid 941 is H, amino acid 956 is K, amino acid 968 is F, amino acid 326 is D, amino acid 358 is L, amino acid 360 is P, amino acid 362 is F, amino acid 404 is N, amino acid 427 is K, amino acid 433 is S, amino acid 464 is N, amino acid 465 is K, amino acid 471 is A, amino acid 480 is R, amino acid 485 is R, amino acid 488 is Y, and amino acid 492 is Y, as compared to the glycoprotein of SEQ ID NO: 2 (referred to as “BA.5 pseudovirus”); and (iii) a SARS-CoV-2 S glycoprotein of the Omicron XBB.1.5 subtype (referred to as “XBB.1.5 pseudovirus”).

HEK293T cells expressing ACE2 (HEK293T/ACE2 cells) were added to the mixture of pseudovirus and sample and incubated for three days to allow non-neutralized viruses to infect the HEK293T/ACE2 cells. On day four, cells were lysed and the luminescence of the cells (relative light units) was measured to determine the level of infection that occurred in the presence of antibodies in serum. The parameters of assay precision, specificity, LLOQ, and ULOQ were evaluated.

Precision: Precision is the closeness of agreement (degree of scatter) between a series of measurements obtained from multiple testing of the same sample. Total or overall precision is composed of intra-assay and inter-assay precision components. Intra-assay (within run) precision is the closeness of multiple determinations of a single sample within one assay run under the same operating conditions over a short interval of time. Inter-assay (between run) precision is the closeness of repeated measurements within laboratory taking into account all relevant sources of variation affecting the results (e.g., runs, analysts, equipment, and reagents).

Intra-assay and inter-assay precision of the PNT assay were determined using a panel of 40 serum samples ranging from negative, low to high PNT titer for each pseudovirus. The assay precision data were generated by 2 analysts on 3 different days in 6 runs, with each sample tested twice in each run. A total of 24 results from 6 runs were generated and used for precision evaluation. In 9 samples, less than 24 (16 to 23) values were available for precision evaluation due to inability to generate titer or insufficient sample volume to test in all runs.

The intra- and inter-assay precision were estimated by calculating percent geometric coefficient of variation (% GCV) using the variance component analysis model with sample as fixed effect and analyst and day as random effects.

The intra-and inter-assay % GCV is calculated based on the natural log-transformed values of PNT titers:

$$ln SD 1 = exp ( \sqrt{var ( Analyst ) + var ( Day )}$$

$$ln SD 2 = exp ( \sqrt{var ( Error )}$$

$$\% GCV ( Inter - Assay ) = 100 \times ( \sqrt{{exp ( ( ln SD 1 ) )}^{2} ) - 1 )}$$

$$\% GCV ( Intra - Assay ) = 100 \times ( \sqrt{{exp ( ( ln SD 2 ) )}^{2} ) - 1 )}$$

Total % GCV for the intermediate precision is calculated as follows:

$$ln SD 3 = exp ( \sqrt{var ( Analyst ) + var ( Day ) + var ( residual ) )}$$

$$Total \% GCV ( Intermediate Precision ) = 100 \times ( \sqrt{{exp ( ( ln SD 3 ) )}^{2} ) - 1 )}$$

The intra-, inter-assay, and total assay precision were evaluated at the individual sample level and also at the strain level, which is the overall assay variance of all 40 samples tested.

Specificity/Selectivity: Specificity is the ability of an analytical method to measure and differentiate the analyte in the presence of components that may be expected to be present. Selectivity is the extent to which the method can determine a particular compound in the analyzed matrices without interference from matrix components. Selectivity of the method is demonstrated by analyzing negative control samples of the appropriate biological matrix (e.g., serum) from multiple sources. Negative control human serum samples from pre-SARS-CoV-2 pandemic time included sera from an influenza vaccine study and RSV vaccine study that showed very strong specific immune responses to influenza virus and RSV F protein as well as normal human serum samples collected pre-SARS-CoV-2 pandemic era were tested.

Linearity: The linearity of an analytical method measures its ability (within a given range) to obtain test results which are directly proportional to the concentration (amount) of analyte in the sample. The linearity of the PNT assay in Wuhan prototype virus was assessed by testing two SARS-CoV-2 PNT-positive samples undiluted and diluted in negative serum. The number of dilutions and dilution factor were dependent on the titer of the undiluted serum (i.e., 1:2, 1:8, more or less dilutions were performed as appropriate and feasible). The linearity samples were tested twice in the same run in duplicates across a total of 6 runs by 2 different analysts on 3 days. The samples were diluted in negative serum independently for each dilution and then heat inactivated.

A simple linear regression was performed on the dataset. The independent variable is log10 expected PNT titer and the dependent variable is log10 observed PNT titer. The point estimate and the 95% confidence interval (CI) of the slope and coefficient of determination (R²) of the regression lines were evaluated.

The expected PNT titers at each dilution were calculated from the overall GMT from all runs of the undiluted sample divided by the dilution factor for each dilution of each sample. The observed PNT titers at each dilution were the overall GMT at each dilution for each sample in all runs.

The accuracy of an analytical procedure is defined as the closeness of agreement between the detected value and the value that is accepted either as a conventional true value or an accepted reference value. The accuracy was evaluated in the linearity study by comparing the expected and the observed values and calculating the percent relative bias. The percent relative bias at each dilution:

$$\% Relative Bias = 100 \times \frac{Observed overall PNT GMT - Expected PNT titer}{Expected PNT titer}$$

A percent relative bias of 150% or −60% corresponds to 2.5-fold higher or 2.5-fold lower, respectively, than the expected titer.

PNT Assay Lower Limit of Quantitation and Upper Limit of Quantitation: The lower limit of quantitation (LLOQ) and upper limit of quantitation (ULOQ) of the PNT assay are the lowest and highest PNT titers that can be quantitatively determined with acceptable precision and accuracy, respectively. The LLOQ and ULOQ were determined using the samples for the linearity evaluation. The precision was estimated by calculating the % GCV of PNT titers across the runs for these samples, and the accuracy was estimated by calculating the percent relative bias across the runs for these samples. Precision and accuracy for the LLOQ determination was evaluated at dilutions where expected PNT titer was between 20-100.

Results: Precision: The tables below contain the precision for the pseudovirus based neutralization assay for pseudoviruses expressing each SARS-CoV-2 S glycoprotein.

Summary of Wuhan D614 Prototype Virus PNT Assay Precision

| | | PNT | Inter- | Intra- | |
|---|---|---|---|---|---|
| | | Titer | assay % | assay % | Total % |
| Sample ID | N³ | GMT | GCV | GCV | GCV |
| Overall¹ | N/A | N/A | 6.6 | 42.8 | 43.4 |
| HMN865526 | 24 | 4048.7 | 46.6 | 34.2 | 60.0 |
| 69-2022-003 | 24 | 1025.5 | 12.9 | 42.4 | 44.7 |
| 15-2023-015 | 24 | 80.6 | 39.8 | 33.2 | 53.4 |
| BRH1240371 | 24 | 20.0² | 0.0 | 0.0 | 0.0 |
| HMN865527 | 24 | 104.0 | 0.0 | 35.9 | 35.9 |
| HMN865528 | 24 | 281.8 | 10.9 | 25.5 | 27.9 |
| HMN865532 | 24 | 12248.5 | 9.0 | 35.5 | 36.8 |
| HMN865542 | 24 | 172.3 | 0.0 | 41.8 | 41.8 |
| HMN865544 | 24 | 193.9 | 0.0 | 40.4 | 40.4 |
| HMN865545 | 24 | 11589.0 | 6.6 | 40.7 | 41.4 |
| HMN865549 | 24 | 8463.1 | 12.4 | 36.2 | 38.6 |
| HMN865551 | 24 | 2128.9 | 25.9 | 56.4 | 63.7 |
| HMN865552 | 23 | 28.0 | 0.0 | 49.4 | 49.4 |
| HMN865554 | 24 | 816.0 | 7.3 | 37.8 | 38.6 |
| HMN865557 | 24 | 2424.7 | 13.8 | 34.5 | 37.5 |
| HMN865561 | 24 | 397.0 | 0.0 | 43.9 | 43.9 |
| HMN865573 | 24 | 2269.5 | 10.6 | 43.6 | 45.1 |
| HMN865574 | 24 | 375.9 | 13.5 | 38.4 | 41.0 |
| HMN865575 | 24 | 783.7 | 3.6 | 43.2 | 43.4 |
| HMN935011 | 24 | 1978.5 | 44.8 | 49.4 | 70.3 |
| HMN935012 | 24 | 1322.5 | 18.1 | 42.9 | 47.2 |
| HMN935018 | 24 | 3044.9 | 50.8 | 41.0 | 68.5 |
| HMN935019 | 24 | 5885.3 | 38.9 | 36.3 | 55.1 |
| HMN865539 | 24 | 1330.1 | 0.0 | 52.9 | 52.9 |
| HMN865539 1:2 | 24 | 546.9 | 37.8 | 33.0 | 51.7 |
| HMN865539 1:8 | 22 | 230.2 | 27.5 | 25.2 | 37.9 |
| HMN865539 1:32 | 18 | 73.0 | 60.5 | 39.6 | 76.1 |
| HMN865539 1:128 | 21 | 27.8 | 17.8 | 36.6 | 41.2 |
| HMN865539 1:256 | 22 | 20.4 | 3.0 | 4.7 | 5.6 |
| HMN865539 1:512 | 24 | 32.8 | 24.6 | 50.6 | 57.6 |
| HMN865539 1:1024 | 24 | 34.4 | 45.8 | 49.3 | 71.0 |
| HMN865536 | 24 | 14863.5 | 0.0 | 59.8 | 59.8 |
| HMN865536 1:2 | 24 | 5386.6 | 47.2 | 42.6 | 66.7 |
| HMN865536 1:8 | 24 | 2136.1 | 7.6 | 43.5 | 44.3 |
| HMN865536 1:32 | 24 | 468.2 | 0.0 | 40.7 | 40.7 |
| HMN865536 1:128 | 21 | 66.0 | 0.0 | 23.5 | 23.5 |
| HMN865536 1:256 | 19 | 44.1 | 0.0 | 41.1 | 41.1 |
| HMN865536 1:512 | 23 | 42.8 | 0.0 | 46.1 | 46.1 |
| HMN865536 1:1024 | 24 | 38.1 | 31.8 | 46.4 | 58.1 |
| 20/136 (WHO ref std) | 16 | 4680.9 | 0.0 | 49.0 | 49.0 |

¹The overall assay precision with Wuhan. D614 was calculated at the strain level by taking into account all 40 individual samples including positive and negative control samples listed in the table.
²When PNT titer is <20 (MRD), a value of 20 was used for calculation purposes.
³Number of values used for calculation.

The overall precision was calculated by analyzing assay variance from all 40 samples using Wuhan D614 prototype virus. The overall inter-assay, intra-assay, and total assay % GCV were 6.6%, 42.8%, and 43.4% for the pseudovirus neutralization assay utilizing pseudovirus expressing the Wuhan D614 SARS-CoV-2 S glycoprotein, respectively. At the individual sample level, among all 40 samples, the lowest % GCV for inter-, intra-, and total assay precision was 0.0%. The highest % GCV for inter-, intra-, and total assay precision was 60.5%, 59.8%, and 76.1%, respectively. Ninety-five percent (95%, 38 of 40), 90% (36 of 40) and 65% (26 of 40) of samples showed inter-, intra-, and total % GCV less than 50, respectively; and 97.5% (39 of 40), 100% (40 of 40) and 85% (34 of 40) of samples showed inter-, intra-, and overall % GCV less than or equal to 60, respectively. It is acceptable that at least 80% samples have % GCV less than 60 for Wuhan D614 virus.

Summary of BA.5 Subvariant Virus PNT Assay Precision

| | | PNT | Inter- | Intra- | |
|---|---|---|---|---|---|
| | | Titer | assay % | assay % | Total % |
| Sample ID | N³ | GMT | GCV | GCV | GCV |
| Overall¹ | N/A | N/A | 6.6 | 42.8 | 43.4 |
| HMN865536 (PC1) | 24 | 4884.5 | 48.2 | 26.9 | 56.7 |
| 35-2023-005 (PC2) | 24 | 1181.2 | 35.5 | 20.4 | 41.6 |
| 69-2022-001 (PC3) | 24 | 122.3 | 35.0 | 16.8 | 39.2 |
| BRH1240371 (NC) | 24 | 20.0 ² | 0.0 | 0.0 | 0.0 |
| HMN865529 | 24 | 20.0 ² | 0.0 | 0.0 | 0.0 |
| HMN865532 | 24 | 434.2 | 9.6 | 24.6 | 26.6 |
| HMN865533 | 24 | 708.5 | 22.2 | 23.5 | 32.8 |
| HMN865534 | 24 | 1000.6 | 25.5 | 40.6 | 49.0 |
| HMN865537 | 24 | 2044.7 | 24.7 | 25.2 | 35.9 |
| HMN865539 | 24 | 603.1 | 14.1 | 19.4 | 24.2 |
| HMN865544 | 24 | 135.4 | 15.8 | 14.2 | 21.4 |
| HMN865545 | 24 | 2490.8 | 25.8 | 41.0 | 49.6 |
| HMN865549 | 24 | 447.5 | 22.1 | 17.0 | 28.1 |
| HMN865550 | 24 | 4486.2 | 34.9 | 16.4 | 39.0 |
| HMN865551 | 24 | 588.4 | 37.2 | 17.2 | 41.5 |
| HMN865556 | 24 | 162.6 | 16.6 | 18.6 | 25.1 |
| HMN865558 | 24 | 212.4 | 8.4 | 14.6 | 16.9 |
| HMN865564 | 24 | 20.0 ² | 0.0 | 0.0 | 0.0 |
| HMN865573 | 24 | 1566.1 | 26.5 | 17.0 | 31.8 |
| HMN865574 | 24 | 132.8 | 0.5 | 14.3 | 14.3 |
| HMN865575 | 24 | 123.4 | 23.3 | 13.6 | 27.2 |
| HMN934981 | 24 | 153.5 | 9.0 | 15.1 | 17.6 |
| HMN935011 | 24 | 1916.8 | 12.3 | 30.0 | 32.6 |
| HMN935012 | 24 | 340.3 | 2.7 | 26.8 | 27.0 |
| HMN935018 | 24 | 1352.6 | 14.8 | 40.3 | 43.3 |
| HMN943977 | 24 | 5228.0 | 0.0 | 46.3 | 46.3 |
| HMN943977 1:2 | 24 | 2391.2 | 12.0 | 46.0 | 47.9 |
| HMN943977 1:8 | 24 | 329.3 | 5.3 | 36.1 | 36.6 |
| HMN943977 1:32 | 20 | 74.7 | 17.7 | 14.7 | 23.1 |
| HMN943977 1:96 | 22 | 40.4 | 16.1 | 33.7 | 37.7 |
| HMN943977 1:192 | 22 | 20.1 | 0.3 | 2.0 | 2.0 |
| HMN943977 1:384 | 22 | 20.0 ² | 0.0 | 0.0 | 0.0 |
| ZA018-0389 | 24 | 28022.7 | 63.1 | 28.5 | 71.5 |
| ZA018-0389 1:2 | 24 | 15856.3 | 28.0 | 24.1 | 37.6 |
| ZA018-0389 1:8 | 24 | 3214.2 | 28.2 | 23.9 | 37.6 |
| ZA018-0389 1:40 | 24 | 317.7 | 0.0 | 34.5 | 34.5 |
| ZA018-0389 1:160 | 20 | 65.5 | 6.2 | 11.6 | 13.2 |
| ZA018-0389 1:320 | 21 | 48.5 | 12.3 | 17.5 | 21.5 |
| ZA018-0389 1:640 | 20 | 35.7 | 11.4 | 48.3 | 49.9 |
| ZA018-0389 1:1280 | 24 | 20.0 ² | 0.0 | 0.0 | 0.0 |

¹The overall assay precision with BA.5 was calculated at the strain level by taking into account all 40 individual samples including positive and negative control samples listed in the table.
² When PNT titer is <20 (MRD), a value of 20 was used for calculation purposes.
³Number of values used for calculation.

The overall precision was calculated by analyzing assay variance from all 40 samples using the BA.5 Subvariant pseudovirus. The overall inter-assay, intra-assay, and total assay % GCV were 15.1%, 26.4%, and 30.7%, respectively. At the individual sample level, among all 40 samples, the lowest % GCV for inter-, intra-, and total assay precision was 0.0%. The highest % GCV for inter-, intra-, and total assay precision was 63.1%, 48.3%, and 71.5%, respectively. Thirty-nine of the forty samples (98%), all forty samples (100%), and thirty-eight of the forty samples (95%) showed inter-, intra-, and overall % GCV less than 50, respectively. Assay precision met the targeted acceptance criteria.

Summary of XBB.1.5 Subvariant Virus PNT Assay Precision

| | | PNT | Inter- | Intra- | |
|---|---|---|---|---|---|
| | | Titer | assay % | assay % | Total % |
| Sample ID | N ³ | GMT | GCV | GCV | GCV |
| Overall ¹ | N/A | N/A | 20.0 | 29.5 | 36.1 |
| HMN865537 (PC1) | 24 | 1533.5 | 28.3 | 45.4 | 55.0 |
| 35-2023-002 (PC2) | 24 | 252.0 | 29.0 | 34.2 | 45.9 |
| 35-2023-010 (PC3) | 24 | 54.4 | 2.1 | 15.1 | 15.3 |
| HMN865529 (NC) | 24 | 20.0 ² | 0.0 | 0.0 | 0.0 |
| HMN865526 | 24 | 149.8 | 0.0 | 15.0 | 15.0 |
| HMN865533 | 24 | 324.6 | 10.2 | 32.1 | 33.8 |
| HMN865534 | 24 | 1004.6 | 24.3 | 35.6 | 43.9 |
| HMN865536 | 24 | 258.4 | 0.0 | 23.5 | 23.5 |
| HMN865539 | 17 | 61.9 | 18.8 | 15.5 | 24.5 |
| HMN865545 | 24 | 1399.6 | 21.7 | 25.2 | 33.8 |
| HMN865549 | 24 | 300.0 | 12.6 | 28.2 | 31.1 |
| HMN865552 | 24 | 20.0 ² | 0.0 | 0.0 | 0.0 |
| HMN865556 | 18 | 45.0 | 14.7 | 20.5 | 25.4 |
| HMN865565 | 24 | 88.8 | 7.3 | 22.5 | 23.7 |
| HMN865573 | 24 | 1000.1 | 35.4 | 26.0 | 44.9 |
| HMN865574 | 23 | 178.9 | 14.8 | 25.2 | 29.5 |
| HMN934992 | 24 | 121.6 | 0.0 | 16.4 | 16.4 |
| HMN935011 | 24 | 177.5 | 8.6 | 12.4 | 15.1 |
| HMN935018 | 24 | 814.1 | 35.0 | 29.2 | 46.7 |
| HMN935019 | 24 | 849.7 | 23.4 | 34.6 | 42.5 |
| 35-2023-012-A | 24 | 6243.8 | 65.7 | 24.8 | 72.1 |
| 35-2023-012-B | 24 | 6725.8 | 41.3 | 31.6 | 53.6 |
| 35-2023-012-C | 24 | 3313.0 | 48.9 | 34.9 | 62.5 |
| 35-2023-012-D | 24 | 1045.1 | 2.3 | 38.5 | 38.5 |
| 35-2023-012-E | 24 | 249.5 | 29.6 | 26.9 | 40.7 |
| 35-2023-012-F | 24 | 253.8 | 18.4 | 18.8 | 26.5 |
| HMN865550 | 24 | 2949.2 | 28.9 | 28.0 | 41.1 |
| HMN865550 1:2 | 24 | 746.0 | 21.6 | 55.9 | 61.1 |
| HMN865550 1:4 | 24 | 366.4 | 41.5 | 36.9 | 57.7 |
| HMN865550 1:12 | 24 | 103.6 | 22.3 | 17.5 | 28.6 |
| HMN865550 1:36 | 24 | 37.5 | 36.3 | 33.1 | 50.6 |
| HMN865550 1:72 | 23 | 20.0 ² | 0.0 | 0.0 | 0.0 |
| HMN865550 1:144 | 24 | 20.0 ² | 0.0 | 0.0 | 0.0 |
| HMN934977 | 24 | 7561.0 | 43.3 | 19.8 | 48.4 |
| HMN934977 1:2 | 24 | 3495.3 | 34.9 | 31.1 | 47.9 |
| HMN934977 1:6 | 24 | 623.9 | 0.0 | 44.3 | 44.3 |
| HMN934977 1:18 | 24 | 171.8 | 27.4 | 18.8 | 33.6 |
| HMN934977 1:54 | 24 | 53.1 | 11.2 | 13.8 | 17.8 |
| HMN934977 1:108 | 22 | 34.3 | 53.4 | 25.7 | 60.8 |
| HMN934977 1:216 | 23 | 20.7 | 2.7 | 15.9 | 16.1 |

¹ The overall assay precision with XBB.1.5 was calculated at the strain level by taking into account all 40 individual samples including positive and negative control samples listed in the table.
² When PNT titer is <20 (MLD), a value of 20 was used for calculation purposes.
³ Number of individual PNT titer values used for calculation.

The overall precision was calculated by analyzing assay variance from all 40 samples using the XBB.1.5 Subvariant pseudovirus. The overall inter-assay, intra-assay, and total assay % GCV were 20.0%, 29.5%, and 36.1%, respectively. At the individual sample level, among all 40 samples, the lowest % GCV for inter-, intra-, and total assay precision was 0.0%. The highest % GCV for inter-, intra-, and total assay precision was 65.7%, 55.9%, and 72.1%, respectively. Thirty-eight (95%), thirty-nine (97.5%) and thirty-two (80%) of the forty samples showed inter-, intra-, and overall % GCV less than 50, respectively. Assay precision met the targeted acceptance criteria.

Results-Specificity: The assay demonstrated specificity for detecting neutralizing antibodies to SARS-CoV-2 antigens. Human serum collected in the pre-SARS-CoV-2 pandemic era had no detectable neutralizing antibodies against the Wuhan D614 pseudovirus, XBB1.5 pseudovirus, or BA.5 pseudovirus (<LLOQ). In addition, 5 pairs of pre- and post-vaccination sera from an RSV F vaccine phase 3 clinical trial RSV-M-301 and 5 pairs of pre- and post-vaccination sera from ananoparticle influenza vaccine phase 3 clinical trial qNIV-E-301 that were collected pre-SARS-CoV-2 pandemic were tested in PNT assay. All samples tested negative (<LLOQ) in Wuhan D614, XBB1.5, and BA.5 PNT assay while having very strong specific immune responses to influenza or RSV at post-vaccination, demonstrating specificity of SARS-CoV-2 PNT assay.

Results-Linearity: The percent relative bias, LLOQ, and ULOQ of pseudoneutralization antibody titer was determined for each pseudovirus. The accuracy of the PNT assay was evaluated by the percent relative bias (% RB). The precision was determined by the % GCV of values from all runs for the dilution point.

Percent Relative Bias, LLOQ and ULOQ Evaluation
of PNT using Wuhan D614 Pseudovirus

| | | | | | % | Inter- | Intra- | |
|---|---|---|---|---|---|---|---|---|
| | | | Observed | Expected | Relative | assay | assay | Total |
| Sample | Dilution | N¹ | PNT GMT | PNT GMT | Bias | % GCV | % GCV | % GCV |
| HMN865539 | 1 | 12 | 1330.0 | 1330.0 | 0.0 | 0.0 | 52.9 | 52.9 |
| | 2 | 12 | 546.8 | 665.0 | −17.8 | 37.8 | 33.0 | 51.7 |
| | 8 | 11 | 230.0 | 166.3 | 38.3 | 27.5 | 25.2 | 37.9 |
| | 32 | 9 | 73.1 | 41.6 | 75.8 | 60.5 | 39.6 | 76.1 |
| | 128 | 12 | 28.6 | 10.4 | 175.1 | 17.8 | 36.6 | 41.2 |
| | 256 | 12 | 20.6 | 5.2 | 296.9 | 3.0 | 4.7 | 5.6 |
| | 512 | 12 | 32.7 | 2.6 | 1157.1 | 24.6 | 50.6 | 57.6 |
| | 1024 | 12 | 34.5 | 1.3 | 2556.8 | 45.8 | 49.3 | 71.0 |
| HMN865536 | 1 | 12 | 14863.2 | 14863.2 | 0.0 | 0.0 | 59.8 | 59.8 |
| | 2 | 12 | 5386.6 | 7431.6 | −27.5 | 47.2 | 42.6 | 66.7 |
| | 8 | 12 | 2136.2 | 1857.9 | 15.0 | 7.6 | 43.5 | 44.3 |
| | 32 | 12 | 468.2 | 464.5 | 0.8 | 0.0 | 40.7 | 40.7 |
| | 128 | 12 | 66.7 | 116.1 | −42.6 | 0.0 | 23.5 | 23.5 |
| | 256 | 12 | 39.9 | 58.1 | −31.3 | 0.0 | 41.1 | 41.1 |
| | 512 | 12 | 41.6 | 29.0 | 43.4 | 0.0 | 46.1 | 46.1 |
| | 1024 | 12 | 38.0 | 14.5 | 161.9 | 31.8 | 46.4 | 58.1 |

NOTE:
Data points with the expected titer at and above 20 are used for plotting the linear regression curve. PNT titer of <20 (MRD) is defined as 20 for calculation purposes.
¹Number of values used for calculation.

Summary of Linearity Regression Parameters for
PNT Assay with Wuhan D614 Prototype Pseudovirus

| Sample | Parameter | Estimate | 95% LCL | 95% UCL |
|---|---|---|---|---|
| HMN865539 | Slope | 0.801 | 0.521 | 1.080 |
| | Intercept | 0.563 | −0.140 | 1.266 |
| | Residual | 18.89 | N/A | |
| | Variability | | | |
| | (% GSD) | | | |
| | R² | 0.987 | N/A | |
| HMN865536 | Slope | 1.000 | 0.847 | 1.153 |
| | Intercept | −0.046 | −0.492 | 0.400 |
| | Residual | 42.32 | N/A | |
| | Variability | | | |
| | (% GSD) | | | |

| | | |
|---|---|---|
| R² | 0.983 | N/A |

NOTE:
LCL = lower confidence limit;
UCL = upper confidence limit

The tables above show the linearity testing data for the Wuhan D614 prototype pseudovirus assay. The linearity testing data for the Wuhan D614 prototype pseudovirus assay showed acceptable linear response. The % RB for all 3 HMN865539 dilution points that had the expected GMT above 20 was within the targeted range (−60% to 150%); % RB for all 6 of HMN865536 dilution points that were above the expected GMT of 20 was within the targeted range. The % GCV for 6 of the 8 dilution points (75%) for sample HMN865539 was <60 and the % GCV for 7 of the 8 dilutions points (87.5%) for sample HMN865536 was <60. The point estimate of the slopes of the linear regression plots for samples HMN865539 and HMN865536 in the linearity tests were 0.801 and 1.000, respectively. The lower confidence limit (LCL) and upper confidence limit (UCL) of 95% confidence interval of slope for sample HMN865539 was 0.521-1.080, outside the targeted range of 0.7-1.43. This could be attributed to the lower titer of the sample and fewer dilution points (only 4 dilution points) that were used for the regression plot. For sample HMN865536, which had 7 dilution points for the regression plot, had 95% LCL and UCL of 0.847-1.153, within the acceptable range of 0.7-1.43. The R² of the regression plots were 0.987 and 0.983 for samples HMN865539 and HMN865536, respectively. The linearity testing data demonstrated acceptable assay linear response and that the method can measure Wuhan prototype pseudovirus neutralizing antibody titers with acceptable precision and relative accuracy. At the expected PNT titer 20 and above, the % RB was within −42.6% and 75.8% for both samples, within a 2.5-fold difference of the expected titers (−60% to 150%). The % GCV at expected GMT of 41.6 for sample HMN865539 was 60.5 and 39.6 for inter- and intra-assay precision, respectively, although the total % GCV was higher (76.1). For sample HMN865536, at the expected GMT of 29.0 and observed GMT of 41.6, the inter-, intra-assay % GCV was 0 and 46.1 respectively, with the total % GCV of 46.1. Therefore, GMT of 42 had acceptable precision (% GCV≤60) and relative accuracy (% RB between −60% and 150%) and is set as the assay LLOQ. The highest PNT GMT titer is 14863.2 with inter-, intra and total GCV less than 60. Therefore, the assay provisional ULOQ is at 14863 for the PNT assay using Wuhan prototype virus. Assay ULOQ may be further evaluated during clinical testing when higher titer serum samples become available.

Percent Relative Bias, LLOQ and ULOQ Evaluation
of PNT Assay with BA.5 Pseudovirus

| | | | | | Inter- | Intra- | |
|---|---|---|---|---|---|---|---|
| | | Observed | Expected | % Relative | assay | assay | Total |
| Sample | Dilution | PNT GMT | PNT GMT | Bias | % GCV | % GCV | % GCV |
| HMN934977 | 1 | 5228.2 | 5228.2 | 0.0 | 0.0 | 46.3 | 46.3 |
| | 2 | 2391.2 | 2614.1 | −8.5 | 12.0 | 46.0 | 47.9 |
| | 8 | 329.4 | 653.5 | −49.6 | 5.3 | 36.1 | 36.6 |
| | 32 | 73.5 | 163.4 | −55.0 | 17.7 | 14.7 | 23.1 |
| | 96 | 40.3 | 54.5 | −26.0 | 16.1 | 33.7 | 37.7 |
| | 192 | 20.1 | 27.2 | −26.2 | 0.3 | 2.0 | 2.0 |
| | 384 | 20.0 | 13.6 | 46.9 | 0.0 | 0.0 | 0.0 |
| ZA018-0389 | 1 | 28022.6 | 28022.6 | 0.0 | 63.1 | 28.5 | 71.5 |
| | 2 | 15856.3 | 14011.3 | 13.2 | 28.0 | 24.1 | 37.6 |
| | | (ULOQ) | | | | | |
| | 8 | 3214.1 | 3502.8 | −8.2 | 28.2 | 23.9 | 37.6 |
| | 40 | 317.7 | 700.6 | −54.7 | 0.0 | 34.5 | 34.5 |
| | 160 | 65.3 | 175.1 | −62.7 | 6.2 | 11.6 | 13.2 |
| | 320 | 48.9 | 87.6 | −44.1 | 12.3 | 17.5 | 21.5 |
| | 640 | 35.5 | 43.8 | −18.9 | 11.4 | 48.3 | 49.9 |
| | | (LLOQ) | | | | | |
| | 1280 | 20.0 | 21.9 | −8.6 | 0.0 | 0.0 | 0.0 |

Note:
Data points with the expected titer at and above 20 are used for plotting the linear regression curve. PNT titer of <20 (MRD) is defined as 20 for calculation purposes.

Summary of Linearity Regression Parameters
for PNT Assay with BA.5 Pseudovirus

| Sample | Parameter | Estimate | 95% LCL | 95% UCL |
|---|---|---|---|---|
| HMN934977 | Slope | 1.063 | 0.872 | 1.253 |
| | Intercept | −0.318 | −0.830 | 0.194 |
| | Residual | 38.3 | N/A | |
| | Variability | | | |
| | (% GSD) | | | |
| | R² | 0.984 | N/A | |
| ZA018-0389 | Slope | 1.107 | 0.959 | 1.256 |
| | Intercept | −0.476 | −0.944 | −0.008 |
| | Residual | 43.2 | N/A | |
| | Variability | | | |
| | (% GSD) | | | |

| | | |
|---|---|---|
| R² | 0.987 | N/A |

The tables above show the linearity testing data for the BA.5 pseudovirus assay. The linearity analysis of the BA.5 PNT assay was performed using 2 serum samples. The point estimate of the slopes of all the linear regression plots in the linearity tests were 1.063 and 1.107, respectively. The lowest lower confidence limit (LCL) of 95% confidence interval of slopes was 0.872. The highest upper confidence limit (UCL) of 95% confidence interval of slopes was 1.256. The slope for both samples passed acceptance criteria of 95% CI 0.7-1.43. The R² of the regression plots were 0.984 and 0.987, respectively. The percentage of relative bias (% RB) for HMN934977 at 6 dilutions was between −60% and 150% that passed acceptance criteria. Six (6) out of seven (7) dilutions (85.7% samples) of ZA018-0389 have % RB between −60% and 150%. It met the acceptance criteria. Since 12 out of the 13 samples (dilutions) tested showed percent relative bias between −60% and 150%, slopes and R² from 2 linearity samples passed acceptance criteria, the method is considered to be able to measure omicron BA.5 pseudovirus neutralization within 2.5-fold of the expected PNT titers. The lower limit of quantitation (LLOQ) and upper limit of quantitation (ULOQ) of the PNT assay are the lowest and highest PNT titers that can be quantitatively determined with acceptable precision and accuracy, respectively. The LLOQ and ULOQ were determined using the samples for the linearity evaluation. The precision was estimated by calculating the % GCV of PNT titers across the runs for these samples, and the accuracy was estimated by calculating the percent relative bias across the runs for these samples. Precision and accuracy for the LLOQ determination was evaluated at dilutions where expected PNT titer was between 20-100.

The accuracy and precision of 2 linearity samples for BA.5 virus PNT assay was evaluated. At the expected PNT titer 20 and above, the percent relative bias was within −55% and −8.2% for both samples, within a 2.5-fold difference of the expected titers (−60% to 150%), except one sample (12 out of 13) with −62.7% relative bias. At 14 out of 15 dilutions (from both samples) the % GCV was <50.0%. The lowest expected PNT GMT titers above 20 were 27.2 (from HMN934977) and 21.9 (from ZA018-0389), respectively. They were related to HMN934977 1:192, ZA018-0389 1:1280 dilutions. At these 2 dilutions, 91.3% (21 out of 23) GMT titers (Appendix 3) were less than 20. They were defined as 20 when % GCV and % RB were calculated, so they had the lowest variability of all. They should not be defined as assay LLOQ. The lowest observed GMT titer with reliable % GCV and % RB was 35.5. The highest PNT GMT titer with % GCV less than 50 and % RB between −60 and 150 is 15856.3. A % GCV≤50% is considered acceptable for this type of semi-quantitative assay. Therefore, a LLOQ of 36 and a ULOQ of 15856 for the PNT assay using BA.5 virus are acceptable. Assay ULOQ may be further evaluated during clinical testing when higher titer serum samples become available.

Percent Relative Bias, LLOQ and ULOQ Evaluation
of PNT Assay with XBB.1.5 Pseudovirus

| | | | Overall | | | Inter- | Intra- | |
|---|---|---|---|---|---|---|---|---|
| | | | Observed | Expected | % Relative | assay | assay | Total |
| Sample | Dilution | N³ | PNT GMT | PNT GMT | Bias | % GCV | % GCV | % GCV |
| HMN865550¹ | 1 | 1 | 2949.3 | 2949.3 | 0.0 | 28.9 | 28.0 | 41.1 |
| | | 2 | | | | | | |
| | 2 | 1 | 745.9 | 1474.6 | −49.4 | 21.6 | 55.9 | 61.1 |
| | | 2 | | | | | | |
| | 4 | 1 | 366.6 | 737.3 | −50.3 | 41.5 | 36.9 | 57.7 |
| | | 2 | | | | | | |
| | 12 | 1 | 103.8 | 245.8 | −57.8 | 22.3 | 17.5 | 28.6 |
| | | 2 | | | | | | |
| | 36 | 1 | 37.4 | 81.9 | −54.3 | 36.3 | 33.1 | 50.6 |
| | | 2 | (LLOQ) | | | | | |
| | 72 | 1 | 20.02 | 41.0 | −51.2 | 0.0 | 0.0 | 0.0 |
| | | 2 | | | | | | |
| | 144 | 1 | 20.02 | 20.5 | −2.3 | 0.0 | 0.0 | 0.0 |
| | | 2 | | | | | | |
| HMN934977² | 1 | 1 | 7560.9 | 7560.9 | 0.0 | 43.3 | 19.8 | 48.4 |
| | | 2 | (ULOQ) | | | | | |
| | 2 | 1 | 3495.2 | 3780.5 | −7.5 | 34.9 | 31.1 | 47.9 |
| | | 2 | | | | | | |
| | 6 | 1 | 623.9 | 1260.2 | −50.5 | 0.0 | 44.3 | 44.3 |
| | | 2 | | | | | | |
| | 18 | 1 | 171.7 | 420.1 | −59.1 | 27.4 | 18.8 | 33.6 |
| | | 2 | | | | | | |
| | 54 | 1 | 53.1 | 140.0 | −62.1 | 11.2 | 13.8 | 17.8 |
| | | 2 | | | | | | |
| | 108 | 1 | 35.5 | 70.0 | −49.2 | 53.4 | 25.7 | 60.8 |
| | | 2 | | | | | | |
| | 216 | 1 | 20.6 | 35.0 | −41.1 | 2.7 | 15.9 | 16.1 |
| | | 2 | | | | | | |

Note:
Data points with the expected titer at and above 20 are used for plotting the linear regression curve. PNT titer of <20 is defined as 20 for calculation purposes.
¹Dilution factors of 1-36 were used in linearity regression summary and plots.
²Dilution factors of 1-108 were used in linearity regression summary and plots.
³Number of GMT values w used for calculation.

Summary of Linearity Regression Parameters
for PNT Assay with XBB.1.5 Pseudovirus

| Sample | Parameter | Estimate | 95% LCL | 95% UCL |
|---|---|---|---|---|
| HMN865550 | Slope | 1.179 | 0.879 | 1.479 |
| | Intercept | −0.758 | −1.60 | 0.087 |
| | Residual | 30.85 | N/A | |
| | Variability | | | |
| | (% GSD) | | | |
| | R² | 0.981 | N/A | |
| HMN934977 | Slope | 1.186 | 1.011 | 1.361 |
| | Intercept | −0.773 | −1.29 | −0.255 |
| | Residual | 29.75 | N/A | |
| | Variability | | | |
| | (% GSD) | | | |

| | | |
|---|---|---|
| R² | 0.989 | N/A |

The tables above show the linearity testing data for the XBB.1.5 pseudovirus assay. The linearity analysis of the BA.5 PNT assay was performed using 2 serum samples. The linearity analysis of the XBB.1.5 PNT assay was performed using 2 serum samples. The point estimate of the slopes of all the linear regression plots in the linearity tests were 1.179 and 1.186, respectively. The lower confidence limit (LCL) and upper confidence limit (UCL) of 95% confidence interval of slope for HMN934977 were 1.011 and 1.361, respectively. They passed targeted acceptance criteria (0.70-1.43). The LCL and UCL of 95% confidence interval of slope for HMN865550 were 0.879 and 1.479, respectively. The UCL was slightly higher than the targeted acceptance criteria (1.43) but is considered acceptable (˜32% higher instead of 30% higher than the point estimate of slope). The R² of the regression plots were 0.981 and 0.989, respectively. The percent relative bias (% RB) for HMN865550 was between −60% and 150% that passed acceptance criteria. Five (5) out of six (6) dilutions (83.3% samples) of HMN934977 have % RB between −60% and 150%. It met the acceptance criteria. Since 11 out of the 12 samples tested showed percent relative bias between −60% and 150%, slopes and R² from two linearity samples passed acceptance criteria, the method is considered to be able to measure omicron XBB.1.5 pseudovirus neutralization within 2.5-fold of the expected PNT titers. The lower limit of quantitation (LLOQ) and upper limit of quantitation (ULOQ) of the PNT assay are the lowest and highest PNT titers that can be quantitatively determined with acceptable precision and accuracy, respectively. The LLOQ and ULOQ were determined using the samples for the linearity evaluation. The precision was estimated by calculating the % GCV of PNT titers across the runs for these samples, and the accuracy was estimated by calculating the percent relative bias across the runs for these samples. Precision and accuracy for the LLOQ determination was evaluated at dilutions where expected PNT titer was between 20-100.

A summary of the accuracy and precision of two linearity samples for XBB.1.5 virus PNT assay is described. At dilution 216 of sample HMN934977, 10 out 12 (83.3%) GMT titers were <20 and were defined as 20 for calculation. Therefore, % GCV was low. The PNT GMT titer (20.6) at this dilution was not appropriate to be defined as LLOQ. The next lowest observed GMT titer was 35.5. The inter- and total % GCV were 53.4 and 60.8%, respectively, they didn't pass targeted acceptance criteria (% GCV<50). At dilution 144 of HMN865550, all 12 GMT titers were <20, at dilution 72 of HMN865550, 11 GMT titers were <20 and were defined as 20 for calculation. Therefore, % GCV was low. The PNT GMT titers (20.0) at these 2 dilutions was not appropriate to be defined as LLOQ. The next lowest GMT titer was 37.4. Inter- and intra-assay precision passed acceptance criteria (% GCV<50), but not total % GCV, which was 50.6%. Since GMT was below 100, close to LLOQ. 50.6% GCV is acceptable. Therefore, GMT titer 37 was defined as assay LLOQ. The highest PNT GMT titer is 7560.9 with % GCV (inter-, intra-, and total) less than 50%. A % GCV≤50% is considered acceptable for this type of semi-quantitative assay. A ULOQ of 7561 for the PNT assay using XBB.1.5 virus is acceptable. Assay ULOQ may be further evaluated during clinical testing when higher titer serum samples become available.

Conclusions: The results described show that the pseudoneutralization assays described herein are appropriate for measuring neutralizing antibodies induced by SARS-CoV-2 and variants thereof.

The results obtained from the assay validation experiments demonstrated that the pseudovirus neutralization assay in Wuhan D614 prototype virus was precise and accurate in measuring PNT titers in human serum, with overall assay % GCV≤60%, and percent relative bias within −60% and 150%. The PNT assay performs in a reliable and reproducible manner for a semi-quantitative assessment of PNT antibody titers. The method showed specificity for SARS-CoV-2 virus used in validation. The method is linear for antisera dilution. The LLOQ and ULOQ were established where sample % GCV was below 60 and sample fit was in the linear range. Therefore, the method is suitable for the analysis of samples in clinical trials of SARS-CoV-2 vaccines against Wuhan D614 prototype virus to assess immunogenicity.

The results obtained from the assay validation experiments demonstrated that the pseudovirus neutralization assay in Omicron BA.5 was precise and accurate in measuring PNT titers in human serum, with overall assay % GCV≤50%, and percent relative bias within −60% and 150%. The PNT assay performs in a reliable and reproducible manner for a semi-quantitative assessment of PNT antibody titers. The method showed specificity for SARS-CoV-2 virus used in validation. The method is linear for antisera dilution and the LLOQ, ULOQ were established where sample % GCV was below 50 and sample fit was in the linear range. Therefore, the method is suitable for the analysis of samples from patients that have been administered SARS-CoV-2 vaccines against omicron BA.5 to assess immunogenicity.

The results obtained from the assay validation experiments demonstrated that the pseudovirus neutralization assay in Omicron XBB.1.5 was precise and accurate in measuring PNT titers in human serum, with overall assay % GCV≤50%, and percent relative bias within −60% and 150%. The PNT assay performs in a reliable and reproducible manner for a semi-quantitative assessment of PNT antibody titers. The method showed specificity for SARS-CoV-2 virus used in validation. The method is linear for antisera dilution and the LLOQ, ULOQ were established where sample % GCV was below 50 and sample fit was in the linear range.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world. The following patent documents are incorporated by reference herein in their entireties: International Publication No. 2021/154812; and International Publication No. 2022/203963; International Publication No. 2023/102448.

1-36. (canceled)

37. A pseudovirus encoding:
(i) a SARS-CoV-2 Spike (S) glycoprotein;
(ii) one or more of Rev, Tat, Gag, and Pol proteins; and
(iii) a reporter protein.

38. The pseudovirus of claim 37, encoding from 1 to about 20, from 2 to about 20, from 3 to about 20, from 4 to about 20, from 5 to about 20, from 6 to about 20, from 7 to about 20, from 8 to about 20, from 9 to about 20, from about 10 to about 20, from about 11 to about 20, from about 12 to about 20, from about 13 to about 20, from about 14 to about 20, from about 15 to about 20, from about 16 to about 20, from about 17 to about 20, from about 18 to about 20, from about 19 to about 20, from 1 to about 10, from 2 to about 10, from 3 to about 10, from 4 to about 10, from 5 to about 10, from 6 to about 10, from about 7 to about 10, from about 8 to about 10, from 2 to 5, from 3 to 5, from 4 to about 8, from 5 to about 8, or from about 6 to about 8 SARS-CoV-2 S glycoproteins.

39. The pseudovirus of claim 37, wherein the reporter protein is one of luciferase, a fluorescent protein, and ZsGreen.

40. The pseudovirus of claim 37, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 1, SEQ ID NO: 2, or SEQ ID NO: 3.

41. The pseudovirus of claim 37, wherein the SARS-CoV-2 S glycoprotein has an inactive furin cleavage site.

42. The pseudovirus of claim 41, wherein the SARS-CoV-2 S glycoprotein has an inactive furin cleavage site having the amino acid sequence of QQAQ (SEQ ID NO: 68).

43. The pseudovirus of claim 37, wherein amino acids 973 and 974 of the SARS-CoV-2 S glycoprotein are proline, as compared to a wild-type SARS-CoV-2 S glycoprotein having the amino acid sequence of SEQ ID NO: 2.

44. The pseudovirus of claim 37, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3, 5-13, 15, 17-19, 21, and 23-66.

45. The pseudovirus of claim 37, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3-13, 20, and 22-66.

46. The pseudovirus of claim 37, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 3, 5-13, and 23-66.

47. The pseudovirus of claim 37, wherein the SARS-CoV-2 S glycoprotein is from a SARS-CoV-2 virus or a variant of SARS-CoV-2, the variant of SARS-CoV-2 being a B.1.1.7 SARSCoV-2 strain; a B.1.351 SARS-CoV-2 strain; a P.1 SARS-CoV-2 strain; a Cal.20C SARSCoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; aC.37 SARS-CoV-2 strain; a B.1.621 SARS-CoV-2 strain; or a B.1.1.529 SARS-CoV-2 strain.

48. The pseudovirus of claim 37, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a SARS-CoV-2 S glycoprotein from a SARS-CoV-2 S omicron variant selected from the group consisting of: BA.1, BA.2.12.1, BA.2, BA.3, BA.4, BA.S, XBB.1.5, XBB.2.3, and XBB.1.16.

49. The pseudovirus of claim 37, comprising a Gag protein and a Pol protein, wherein a fusion protein encodes the Gag protein and the Pol protein.

50. The pseudovirus of claim 49, wherein the Gag protein and Pol protein are from a Murine Leukemia Virus (MLV).

51. The pseudovirus of claim 37, wherein the one or more of Rev, Tat, Gag, and Pol proteins are from a lentivirus.

52. A method of producing the pseudovirus of claim 37, comprising transfecting a host cell with:
(i) a transfer plasmid encoding a reporter protein;
(ii) one or more plasmids encoding a Rev protein, a Tat protein, a Gag protein, a Pol protein; or a combination thereof; and
(iii) a plasmid encoding a SARS-CoV-2 Spike (S) glycoprotein.

53. The method of claim 52, wherein the host cell is a human cell or a human or a HEK293T (also called “293T’) cell.

54. A method for determining if a biological sample contains neutralizing antibodies against a SARS-CoV-2 virus or a variant thereof, comprising:
(a) contacting a biological sample with from 1 to about 20 pseudoviruses of claim 37;
(b) contacting a cell expressing angiotensin converting enzyme 2 (ACE2) with the from 1 to about 20 pseudoviruses;
(c) quantifying expression of the reporter protein in the cell expressing ACE2;
(d) contacting a control cell expressing ACE2 with the from 1 to about 20 pseudoviruses, wherein the from 1 to about 20 pseudoviruses have not been contacted with the biological sample; and
(e) quantifying expression of the reporter protein in the control cell expressing ACE2;
wherein if the expression of the reporter protein in the cell of (c) is less than expression of the reporter protein in the control cell of (e), then the biological sample contains neutralizing antibodies against the SARS-CoV-2 virus or the variant thereof.

55. The method of claim 52, comprising contacting the biological sample and pseudovirus for from 1 hour to about 7 hours.

56. The method of claim 52, comprising contacting the biological sample and pseudovirus at about 37° C.

57. The method of claim 52, wherein the cells are selected from the group consisting of: Vero E6, A549, and HEK293T cells.

58. The method of claim 54, wherein the biological sample is i) serum, plasma, or blood from a patient who has been administered an immunogenic composition against a SARS-CoV-2 virus or a variant thereof, or is ii) serum, plasma, or blood from a patient who has previously had COVID-19.
